(19) United States
(12) Patent Application Publication
(10) Pub. No.: US 2023/0295348 A1
(43) Pub. Date: Sep. 21, 2023

(54) COMPOSITION AND METHODS FOR THE SELECTIVE ACTIVATION OF CYTOKINE SIGNALING PATHWAYS

(71) Applicant: Novimmune SA, Geneva (CH)

(72) Inventors: Nicolas FISCHER, Lancy (CH); Julien MONTORFANI, Gland (CH); Limin SHANG, Bellevue (CH); Eric HATTERER, Valleiry (FR)

(21) Appl. No.: 18/158,980

(22) Filed: Jan. 24, 2023

Related U.S. Application Data
(60) Provisional application No. 63/302,514, filed on Jan. 24, 2022.

(51) Int. Cl. C07K 16/46; A61P 35/00

(52) U.S. Cl. CPC C07K 16/468; A61P 35/00; C07K 2317/31; C07K 2317/24

(57) ABSTRACT

The present disclosure provides compositions of bispecific antibodies useful in selective cytokine activation on immune cells. The present disclosure also provides compositions of bispecific antibodies useful for treatment of cancers that express tumor associated antigens.

### RELATED APPLICATIONS

This application claims priority to, and the benefit of, U.S. Application No. 63/302,514, filed on Jan. 24, 2022. The contents of this application are incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is NOVI_049_001US_SeqList_ST26.xml. The XML file is 158,062 bytes in size, created on Jan. 24, 2023, and is being submitted electronically via USPTO Patent Center.

### BACKGROUND OF THE INVENTION

Interleukin-2 (IL-2) is a 15 kDa cytokine essential for the balance between activation and suppression of immune responses. Under normal conditions, CD4+ helper T cells are the main producers of the cytokine. Upon immune activation, both CD4+ and CD8⁺ T cells are induced to produce IL-2 and the induction is regulated through a negative feedback loop involving B lymphocyte-induced maturation protein 1 (BLIMP1)

IL-2 receptor (IL-2R) has three subunits, IL-2Rα (CD25), IL-2Rβ (CD122) and IL-2Rγ (CD132). Of the three IL-2R subunits, only IL-2Rα is unique to IL-2Rβ IL-2Rγ (also called the common γ chain) is common with IL-4, IL-7, IL-9, IL-15 and IL-21 receptor complexes. IL-2Rβ is common with the Interleukin-15 (IL-15) receptor complex. IL-15 is a cytokine for the development, proliferation, and activation of effector NK cells and CD8+ memory T cells. IL-15 binds to the IL-15 receptor α (IL-15Rα) and is presented in trans to the IL-2Rβ-IL2Rγ complex on effector cells. IL-15 and IL-2 share binding to the IL-2Rβ-IL2Rγ complex, and signal through STAT3 and STATS pathways. However, unlike IL-2, IL-15 does not support maintenance of CD4⁺CD25⁺FoxP3⁺ regulatory T (Treg) cells or induce cell death of activated CD8⁺ T cells. Additionally, IL-15 is the only cytokine known to provide anti-apoptotic signaling to effector CD8⁺ T cells. IL-15 signaling exhibits potent anti-tumor activities against well-established solid tumors in experimental animal models.

IL-2 signaling can be transduced through a dimeric intermediate affinity receptor (10⁻⁹ M) composed of IL-2Rβ and IL-2Rγ or through a high affinity trimeric receptor containing all three subunits. The dimeric form is expressed at the surface of memory CD8+ T cells and NK cells. IL-2Rα has low affinity to IL-2 (10⁻⁸ M) and does not mediate signaling by itself. However, in combination with CD122 and CD132, it enhances the binding affinity to IL-2 by 100 folds to form the high affinity (10⁻¹¹ M) trimeric receptor expressed on regulatory CD4+ Foxp3+ T cells (Tregs), activated effector T cells and endothelial cells. As Tregs express the highest level of IL-2Rα under resting conditions, homeostatic low level of IL-2 selectively triggers IL-2 signaling on Treg cells and maintains immune suppression. During immune activation, the elevated level of IL-2 also signals through the dimeric receptor and sustains T cell and NK cell activation and proliferation.

Because of its role in promoting T cell and NK cell activation and proliferation, IL-2 has been pursued as a promising therapeutic target in cancer immunotherapy. High dose of Aldesleukin (a recombinant human IL-2 (hIL-2)) has been approved for the treatment of metastatic renal cell carcinoma and metastatic melanoma with an objective response rate of 17-20% and complete responses lasting up to 91 months. However, the inconvenient short half-life of Aldesleukin intrinsic to its molecular nature as a cytokine and severe toxic effects associated with systemic activation of IL-2 signaling have limited the clinical use of Aldesleukin. The most severe toxicity associated with Aldesleukin is vascular leaking syndrome (VLS), which results in multi-organ edema and damage requiring intensive in-patient monitoring and caring. A preclinical study suggests that VLS is caused by IL-2 signaling in vascular endothelial cells.

To overcome these limitations and exploit the full potential of IL-2 targeting therapies, different new approaches such as hIL-2/mAb conjugates, IL-2 variants with reduced binding to IL-2Ra, PEGylated IL-2 variants, IL-2/IL-2Rα fusion proteins and IL-2Rβ/IL-2Rγ agonistic bispecific antibodies (bsAbs), have been applied to develop molecules with longer half-lives, reduced preferential activation of Tregs and/or more selective IL-2 signaling activation in tumor microenvironments (TME).

The most clinically advanced IL-2 targeting molecule is Bempegaldesleukin, a PEGylated IL-2 variant that is in stage III clinical trials (NCT03635983 and NCT03729245) and has shown promising results so far PEGylation of IL-2 extends its half-life and reduces systemic exposure to high level of active IL-2. However, Bempegaldesleukin still has the potential to induce systemic IL-2 signaling and thus carries the risk of causing systemic toxicity. The hIL-2/mAb conjugates ligates recombinant hIL-2 to a tumor targeting mAb. The introduction of tumor targeting mAb extends the half-life of the molecule and increases tumor specificity. However, the hIL-2 part of the molecule is still active systemically and thus still bears the risk of systemic toxicity. Similarly, IL-2β/IL-2Rγ agonistic bsAbs have an extended half-life and the potential to reduce Treg preferential IL-2 activation. Nonetheless, they still carry the risk of systemic IL-2 activation and associated toxicity. A need exists for composition and methods for selective activation of IL-2.

### SUMMARY OF THE INVENTION

This disclosure provides the use of a composition comprising: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor, for inhibiting tumor growth.

This disclosure provides the use of a composition comprising: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor, for the treatment of cancer.

This disclosure provides the use of a composition comprising: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor, for enhancing T-cell mediated cell killing.

This disclosure provides a composition for use in a method of T-cell activation, wherein the composition comprises: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor, for T-cell activation.

This disclosure provides a composition comprising: a) a first composition comprising a first bispecific antibody comprising an antigen binding domain that binds to a first tumor associated antigen and an antigen binding domain that binds to a first subunit of a cytokine receptor; and b) a second composition comprising a bispecific antibody having an antigen binding domain that binds a second tumor associated antigen and an antigen binding domain that binds to a second subunit of the cytokine receptor.

In some embodiments, the first tumor associated antigen and the second tumor associated antigen are different tumor associated antigens. In some embodiments, the first tumor associated antigen and the second tumor associated antigen are the same tumor associated antigen. In some embodiments, the first tumor associated antigen and the second tumor associated antigen are expressed on the surface of the same tumor cell.

In some embodiments, the first tumor associated antigen and/or the second tumor associated antigen is human epidermal growth factor receptor 2 (HER2). In some embodiments, the first tumor associated antigen and/or the second tumor associated antigen is mesothelin (MSLN).

In some embodiments, the antigen binding domain that binds to the first tumor associated antigen and the antigen binding domain that binds to the second tumor associated antigen, bind to two different epitopes of the same tumor associated antigen. In some embodiments, the antigen binding domain that binds to the first tumor associated antigen and the antigen binding domain that binds to the second tumor associated antigen are identical.

In some embodiments, the first subunit of a cytokine receptor and the second subunit of a cytokine receptor are expressed on the surface of the same immune cell. In some embodiments, the immune cell is a T-cell.

In some embodiments, the cytokine receptor binds IL-2, IL-4, IL-7, IL-9, IL-15, IL-21, IL-12, IL-23, IFNα, IFNβ, IFNε, IFNk, IFNo, IFNδ, IFNτ, IFNω̅, IFNζ, IFNγ or IFNλ.

In some embodiments, the first subunit of the cytokine receptor or the second subunit of the cytokine receptor is a IL-2Rγ. In some embodiments, the first subunit of the cytokine receptor is IL-2Rβ and the second subunit of the cytokine receptor is IL-2Rγ.

In some embodiments, the composition further comprises: c) a third bispecific antibody comprising an antigen binding domain that binds to a third tumor associated antigen and an antigen binding domain that binds to an antigen expressed on a T-cell.

In some embodiments, the third tumor associated antigen is different than the first tumor associated antigen and the second tumor associated antigen. In some embodiments, the antigen expressed on the T-cell is a CD3.

In some embodiments, the first bispecific antibody, the second bispecific antibody and/or the third bispecific antibody has an IgG isotype. In some embodiments, the first bispecific antibody, the second bispecific antibody and/or the third bispecific antibody is a chimeric antibody, a humanized antibody or a human antibody.

In some embodiments, the composition enables antigen dependent activation of the cytokine receptor. In some embodiments, the composition enables antigen dependent activation of IL-2 receptor signaling in immune cells expressing IL-2Rγ and IL-2Rβ. In some embodiments, the composition enables antigen dependent activation of IL-15 receptor signaling in immune cells expressing IL-2Rγ and IL-2Rβ.

The disclosure also provides a method of inhibiting tumor growth or progression in a subject in need thereof, comprising administering to the subject a therapeutically effective amount of any one of the compositions of the disclosure.

The disclosure also provides a method of treating cancer in a subject in need thereof, comprising administering to the subject a therapeutically effective amount of the any one of the compositions of the disclosure.

The disclosure also provides a method of enhancing T-cell mediated cell killing, comprising administering to the subject a therapeutically effective amount of any one of the compositions of the disclosure.

The disclosure also provides a method of enhancing T-cell activation, comprising administering to the subject a therapeutically effective amount of any one of the compositions of the disclosure.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIGS. 1A-1C Schematic representation of the activation of IL-2 signaling by the combination of agonistic anti-IL-2Rβ×anti-TAA bispecific antibody (bsAb) and agonistic anti-IL-2Rγ×anti-TAA bsAb. The inventors describe a new IL-2 agonistic approach using the combination of two bsAbs. The idea behind the concept is to activate IL-2 signaling by mechanically bringing IL-2Rβ and IL-2Rγ into proximity of each other selectively in tumor microenvironments containing TAA positive tumor cells using the combination of IL-2Rβ×TAA bsAb and IL-2Rγ×TAA bsAb. In healthy tissues (i.e., in the absence of cells expressing TAA), the pair of bsAbs can bind to IL-2Rβ and IL-2Rγ on T cells or NK cells respectively but cannot bring the two receptors into proximity and IL-2 signaling is not activated (FIG. 1A). In the presence of TAA positive tumor cells (FIGS. 1B and 1C), binding to TAA by the pair of bsAbs brings the pair of bsAbs into proximity. The subsequent binding of the pair of bsAbs to IL-2Rβ and IL-2Rγ on T cells or NK cells brings the two receptors into proximity of each other, which induces the activation of the IL-2R signaling pathways. The TAA targeting arms of the pair of bsAbs could target the same TAA epitope (FIG. 1B) or two different TAA epitopes (FIG. 1C). This novel strategy of IL-2 signaling activation will limit the agonistic effect to the tumor microenvironments containing TAA positive tumor cells and avoid systemic toxicity. Avoiding preferential activation of CD25 expressing Treg cells is another advantage of this novel approach.

FIGS. 2A-2H. Binding profiles of IL-2β×TAA and IL-2Rγ×TAA bsAbs to their respective targets assessed by ELISA. Concentration dependent binding profiles of IL-2Rβ×HER2 KiH bsAbs (P2C4×Trastuzumab and P2C4× Pertuzumab), IL-2Rγ×HER2 KiH bsAbs (P1A3× Trastuzumab and P1A3× Pertuzumab), and IL-2Rβ×IL-2Rγ KiH bsAbs to their targets IL-2Rβ (FIG. 2A), IL-2Rγ (B), HER2 (FIG. 2C) and to an irrelevant protein (FIG. 2D) assessed by ELISA. Anti-IL-2Rβ arms (AL1 to AL5) were combined to a membrane distal (domain 1) HER2 binding arm (N2-28) and IL-2Rγ arms (AM1 to AM11) were combined to a membrane proximal (domain 4) HER2 arm (N2-19) for κλ-body generation. IL-2Rβ arm (AL4) and IL-2Rγ arm (AM5) were combined to different MSLN arms targeting different epitopes (O30, O35, O38 and O41) in another set of κλ-bodies. Binding profiles of κλ-bodies to IL-2Rβ (FIG. 2E) and IL-2Rγ (FIG. 2F). Binding profiles of IL-2Rβ- and IL-2Rγ-bsAbs to HER2 (FIG. 2G left and right panel respectively) and MSLN (FIG. 2H and right panel respectively) are depicted. hIgG1 was used as an isotype control.

FIGS. 3A-3C. Simultaneous co-engagement of two target antigens by IL-2R×HER2 bsAbs assessed by Octet. Co-engagement was assessed using protein A biosensor loaded with the different bsAbs in the presence of either an individual target or the two targets of the corresponding bsAb simultaneously. Co-engagement of two targets was shown for P1A3× Pertuzumab (FIG. 3A), P2C4× Pertuzumab (FIG. 3B) and P2C4× Trastuzumab (FIG. 3C).

FIGS. 4A-4D: The lack of interference of the IL-2R×TAA bsAbs with IL-2 and IL15 signaling. IL-2 reporter cell line (expressing the dimeric IL-2R) was incubated with recombinant hIL-2 (at 0.2 or 1 nM fixed concentration) or hIL-15 (0.003 nM fixed concentration). Impact of a dose range of IL-2Rβ-κλ bodies (FIGS. 4A-4B) or IL-2Rγ-bodies (FIGS. 4C-4D) on IL-2 or IL-15 signaling was assessed. hIgG1 is used as an irrelevant and non-blocking control antibody. Data were normalized to the isotype control fixed at 100% (absence of neutralization of IL-2/IL-15 signaling, dashed line). Neutralizing IL-2 or IL-15 antibodies were included as controls.

FIGS. 5A-5D. Activation of IL-2 signaling by the combination of IL-2Rβ×HER2 and IL-2Rγ×HER2 KiH bsAbs in the presence of soluble HER2. Schematic representation of the assay format (FIG. 5A). IL-2 reporter cell line was incubated with the combination of IL-2Rβ×HER2 and IL-2Rγ×HER2 bsAbs targeting either the same (FIGS. 5B-5C) or two different (FIG. 5D) HER2 epitopes in the presence of different concentrations of soluble HER2. Activation of IL-2 signaling was manifested by increased expression of the reporter gene. The anti-IL-2Rβ×anti-IL-2Rγ bsAb, P1A3×P2C4, was used as a positive control.

FIGS. 6A-6E. Activation of IL-2 signaling by the combination of IL-2Rβ×HER2 and IL-2Rγ×HER2 KiH bsAbs in the presence of HER2-coated microspheres. Schematic representation of the assay format (FIG. 6A). IL-2 reporter cell line was incubated with the combination of IL-2Rβ×HER2 and IL-2Rγ×HER2 bsAbs targeting either different (FIGS. 6B-6C) or the same (FIGS. 6D-6E) HER2 epitope in the presence of different numbers of HER2-coated streptavidin microspheres. Activation of IL-2 signaling was manifested by increased expression of the reporter gene. Human IL-2 and the anti-IL-2Rβ×anti-IL-2Rγ bsAb, P1A3×P2C4, were used as positive controls.

FIGS. 7A-7I. Activation of IL-2 signaling by the combination of IL-2Rβ×TAA and IL-2Rγ×TAA κλ-bodies in the presence of TAA-coated microspheres. IL-2 reporter cell line was incubated with combination of IL-2Rβ×HER2 and IL-2Rγ×HER2 κλ-bodies targeting two different HER2 epitopes (N2-28 and N2-19 arms) in the presence of HER2-coated streptavidin microspheres (ratio beads-reporter cells, 6:1). Different combinations were depicted where IL-2Rγ×HER2 κλ-bodies (AM1N2-19 to AM5N2-19) were mixed to AL1N2-28 (FIG. 7A), AL2N2-28 (FIG. 7B), AL3N2-28 (FIG. 7C), AL4N2-28 (FIG. 7D) or AL5N2-28 (FIG. 7E). IL-2 reporter cell line was also incubated with various combination of IL-2Rβ×MSLN and IL-2Rγ×MSLN κλ-bodies targeting two different MSLN epitopes (FIGS. 7F-7I) in the presence of MSLN-coated streptavidin microspheres. Different combinations were depicted where IL-2Rγ×MSLN κλ-bodies (AM5O30/N, AM5O35/N, AM5O41/N) were mixed to AL4O38/N (FIG. 7F), AL4O35/N (FIG. 7G), AL4O30/N (FIG. 7H) or AL4O41/N (FIG. 7I). Activation of IL-2 signaling was manifested by increased expression of the reporter gene. Human IL-2 and an irrelevant hIgG1 antibody were used as positive and negative controls respectively.

FIGS. 8A-8C. Activation of IL-2 signaling by the combination of IL-2Rβ×MSLN and IL-2Rγ×MSLN κλ-bodies targeting the same MSLN epitope in the presence of MSLN-coated microspheres. IL-2 reporter cell line was incubated with combination of IL-2Rβ×MSLN and IL-2Rγ×MSLN κλ-bodies targeting the same epitope within MSLN using either O35 (FIG. 8A), O41 (FIG. 8B) or O30 (FIG. 8C) arms. Activation of IL-2 signaling was manifested by increased expression of the reporter gene. Human IL-2 and an irrelevant hIgG1 antibody were used as positive and negative controls respectively.

FIGS. 9A-9C. Activation of IL-2 signaling by the combination of IL-2Rβ×HER2 and IL-2Rγ×HER2 KiH bsAbs in the presence of HER2⁺ tumor cells. IL-2 reporter cell line was incubated with the combination of IL-2Rβ×HER2 and IL-2Rγ×HER2 KiH bsAbs targeting two different HER2 epitopes. Schematic representation of the assay format (FIG. 9A). Combination of P1A3× Trastuzumab+P2C4× Pertuzumab (FIG. 9B) or P1A3× Pertuzumab+P2C4× Trastuzumab (FIG. 9C) were added on HER2+BT-474, SK-BR-3 and NCI-N87 tumor cells. Activation of IL-2 signaling was manifested by increased expression of the reporter gene. Human IL-2 was used as positive control.

FIGS. 10A-10F. pSTAT5 induction by the combination of IL-2111×HER2 and IL-2Rγ×HER2 KiH bsAbs in the presence of HER2⁺ tumor cells. Schematic representation of the assay format (FIG. 10A). NK-92 cell line was incubated with the combination of IL-2Rβ×HER2 and IL-2Rγ×HER2 KiH bsAbs targeting either two different HER2 epitopes (FIGS. 10B-10C) or the same HER2 epitope (FIGS. 10D-10E), in the presence of HER2+BT-474, SK-BR-3 and NCI-N87 tumor cells. IL-2 agonistic properties were also tested by combining all four bsAbs (P1A3× Pertuzumab, P2C4× Pertuzumab, P1A3× Trastuzumab and P2C4× Trastuzumab) (FIG. 10F). IL-2 signaling activation in NK-92 cells was analyzed by measuring the level of pSTAT5 by flow cytometry. Data were presented as the percentage of pSTAT5-positive cells. Human IL-2 was used as a positive control.

FIGS. 11A-11E. pSTAT5 induction by the combination of IL-2R×HER2 or IL-2R×MSLN κλ-bodies in the presence of TAA tumor cells. NK-92 cell line was incubated with the combination of IL-2Rβ×HER2 and IL-2Rγ×HER2 κλ-bodies (FIGS. 11A-11B) or IL-2Rβ×MSLN and IL-2Rγ×MSLN κλ-bodies (FIGS. 11C-11E), in the presence of HER2+BT-474 and NCI-N87 tumor cells (FIGS. 11A and 11B, respectively), or in the presence of MSLN+NCI-H226, OVCAR-3 and NCI-N87 tumor cells (FIGS. 11C, 11D and 11E, respectively). IL-2 signaling activation in NK-92 cells was analyzed by measuring the level of pSTAT5 by flow cytometry. Human IL-2 was used as a positive control. hIgG1 irrelevant antibody (dashed lines) and single treatment with IL-2Rβ×MSLN or IL-2Rγ×MSLN κλ-bodies were used as negative controls.

FIGS. 12A-12B. Activation of IL-2 signaling in human primary T cells by the combination of IL-2β×HER2 and IL-2Rγ×HER2 KiH bsAbs in the presence of HER2⁺ tumor cells.

Schematic representation of the assay format (FIG. 12A). PBMCs were incubated with the combination of IL-2Rβ×HER2 and IL-2Rγ×HER2 KiH bsAbs in the presence of BT-474 or NCI-N87, two different HER2⁺ tumor cell lines (FIG. 12B). IL-2 signaling activation in PBMCs data are presented as the percentage of pSTAT5-positive cells in different T cell subsets gated by FACS: Treg, CD8⁺ and CD4⁺ T cells. Human IL-2 was used as a positive control.

FIGS. 13A-13D. T cell retargeted killing/lysis of tumor cells by the combination of CD3×TAA bsAb and IL-2R×TAA bsAb pairs. Combinations of CD3× TAA bsAb and IL-2R×TAA bsAb pairs were tested for their tumoricidal activity either when combined all together at the same time (FIGS. 13A-13B) or tested sequentially by first pre-stimulating T cells with IL-2R×TAA bsAb pairs followed by the CD3×TAA bsAb (FIGS. 13C-13D). Schematic representations of each TDCC assay format used (FIGS. 13A and 13C). NCI-N87 tumor cell line was used as a source of HER2 and MSLN. T cell retargeted killing/lysis of NCI-N87 cells by a dose range of two of the IL-2R×MSLN bsAb pairs (AL4O35/N+AM5O30/N or AL4O38/N+AM5O30/N) when combined at the same time with a fixed dose of an CD3×HER2 bsAb (FIG. 13B). Sequential T cell retargeted killing/lysis of NCI-N87 cells by a dose range of IL-2 or IL-2R×HER2 bsAb pair (P1A3× Trastuzumab+P2C4× Pertuzumab), followed by a fixed dose of a CD3×MSLN bsAb (FIG. 13D). CD3×TAA single treatment was used as control (dotted line). In some experiments, CD3×TAA bsAb combination with IL-2 was used as a positive control. IL-2Rβ×IL-2Rγ (P1A3×P2C4) or single treatments with IL-2 or IL-2R×TAA bsAbs were used as negative controls. Data were presented using a representative PBMC donor as a source of effector cells.

### DETAILED DESCRIPTION OF THE INVENTION

The invention is based in part on a bispecific antibody platform that can selectively activate cytokines in the tumor microenvironment. Specifically, the invention is based upon activating cytokines whose signaling requires the interaction of two or more cytokine receptor subunits. By combining bispecific antibodies, where each bispecific has one antigen binding arm that is specific for cytokine receptor subunit and one binding arm specific for a cell surface antigens subunit. Binding of the bispecific antibodies to the cell surface antigen brings the different subunits of the cytokine receptor expressed on cell surface (e.g., T-cells, and/or NK-cells) into proximity of each other, which results in cytokine signaling activation. This bispecific antibody (bsAb) platform combines favorable pharmacokinetics properties of bsAb (Le, significantly longer half-lives compared to native or recombinant cytokines) with selective cytokine activation in the tumor microenvironment (TME) to optimally exploit the anti-tumor potential of cytokine activation. Additionally, the bsAb platform of the invention provides a benefit in terms of tumor-specificity and reduced off-tumor systemic toxicity when compared to cytokine receptor activation using native or recombinant cytokines. Further, the invention also avoids preferential activation of cells expressing the trimeric form of the receptor, such as Tregs (e.g., IL-2R trimer which is highly expressed on Tregs), by targeting only the dimeric form of the receptor.

Without being bound by theory, the invention comprises bsAbs that engage cytokine receptors but do not prevent or reduce endogenous cytokine signaling. As therapeutic proteins often induce anti-drug antibodies (ADA), the therapeutic use of either recombinant cytokines such as IL-2 or modified IL-2 peptides cause the potential of inducing such responses in subjects that could also affect endogenous IL-2, which lead to toxic and detrimental effects. Such effects have been observed with Erythropoietin (EPO) immunogenicity, potentially leading to EPO-resistant anemia. A significant advantage of the use of bsAbs of the invention is that any ADA formation would limit the exposure and effectiveness of the bsAbs, but leave endogenous cytokines unaffected. For example, the compositions comprising the bispecific antibodies of the invention do not prevent or inhibit the endogenous signaling induced by IL-2 or IL-15.

Without being bound by theory, the composition engages IL-2Rγ and IL-2Rβ in an antigen dependent manner and does not preferentially activate T regs. The composition also enables T cell activation and proliferation leading to superior killing of target cells. In some embodiments, additional T cell redirection by a third bispecific antibody that binds CD3 on T cells and a tumor antigen on target cells may be used to enhance the composition.

In various aspects, the invention provides composition containing a pair of bsAbs. One of the bsAb targets (i.e., has an antigen binding domain specific for) a first subunit of a multi subunit cytokine receptor and a cell surface protein. The other bsAb targets a second subunit of a multi-subunit cytokine receptor and a cell surface protein expressed on the surface of the same cell that expresses the target antigen of the first bsAb. Cells expressing the cell surface protein bind to the two bsAbs and bring the cytokine receptors on T cells or NK cells into proximity, which results in cytokine signaling activation.

The multi-domain cytokine receptors include for example, cytokine receptors that share a common γ chain, such as IL-2, IL-4, IL-7, IL-9, IL-15 and IL-21 and other cytokine receptors that share another common subunit, such as IL-12 and IL-23. Other multi-domain cytokine receptors include IFNα, IFNβ, IFNε, IFNk, IFNo, IFNδ, IFNτ, IFNω̅, IFNζ, IFNγ, and IFNλ.

The cell surface receptor is a tumor associated antigen (TAA). TAAs are well known in the art and include, for example, a glioma-associated antigen, carcinoembryonic antigen (CEA), EGFRvIII, glypican 3 (GPC3), cMet, IL-IIRa, IL-13Ra, EGFR, FAP, B7H3, Kit, CA LX, CS-1, MUC1, BCMA, bcr-abl, HER2, β.-human chorionic gonadotropin, alphafetoprotein (AFP), ALK, CD19, CD123, lectin-reactive AFP, Fos-related antigen 1, ADRB3, thyroglobulin, EphA2, RAGE-1, RUI, RU2, SSX2, AKAP-4, LCK, OY-TESI, PAXS, SART3, CLL-1, fucosyl GM1, GloboH, MN-CA IX, EPCAM, EVT6-AML, TGSS, polysialic acid, PLAC1, RUI, RU2 (AS), intestinal carboxyl esterase, lewisY, sLe, LY6K, mut hsp70-2, MYCN, RhoC, TRP-2, CYPIBI, BORIS, prostase, prostate-specific antigen (PSA), PAX3, PAP, NY-ESO-1, LAGE-Ia, LMP2, NCAM, Ras mutant, gpIOO, prostein, OR51E2, PANX3, PSMA, PSCA, Her2/neu, hTERT, HMWMAA, HAVCR1, VEGFR2, PDGFR-β, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-AL MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, FAP, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, CLDN18.2, TSHR, UPK2, and mesothelin.

Other antibody domains or tumor target binding proteins useful in the invention (e.g. TCR domains) include, but are not limited to, those that bind the following antigens (note, the cancer indications indicated represent non-limiting examples): aminopeptidase N (CD13), annexin A1, B7-H3 (CD276, various cancers), CA125 (ovarian cancers), CA15-3 (carcinomas), CA19-9 (carcinomas), L6 (carcinomas), Lewis Y (carcinomas), Lewis X (carcinomas), alpha fetoprotein (carcinomas), CA242 (colorectal cancers), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma, B-cell neoplasmas, autoimmune diseases), CD21 (B-cell lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (metastatic melanoma, sarcoma), CD52 (leukemia), CD56 (small cell lung cancers, ovarian cancer, Merkel cell carcinoma, and the liquid tumor, multiple myeloma), CD66e (carcinomas), CD70 (metastatic renal cell carcinoma and non-Hodgkin lymphoma), CD74 (multiple myeloma), CD80 (lymphoma), CD98 (carcinomas), CD123 (leukemia), mucin (carcinomas), CD221 (solid tumors), CD22? (breast, ovarian cancers), CD262 (NSCLC and other cancers), CD309 (ovarian cancers), CD326 (solid tumors), CEACAM3 (colorectal, gastric cancers), CEACAMS (CEA, CD66e) (breast, colorectal and lung cancers), DLL4 (A-like-4), EGFR (various cancers), CTLA4 (melanoma), CXCR4 (CD 184, heme-oncology, solid tumors), Endoglin (CD 105, solid tumors), EPCAM (epithelial cell adhesion molecule, bladder, head, neck, colon, NHL prostate, and ovarian cancers), ERBB2 (lung, breast, prostate cancers), FCGR1 (autoimmune diseases), FOLR (folate receptor, ovarian cancers), FGFR (carcinomas), GD2 ganglioside (carcinomas), G-28 (a cell surface antigen glycolipid, melanoma), GD3 idiotype (carcinomas), heat shock proteins (carcinomas), HER1 (lung, stomach cancers), HER2 (breast, lung and ovarian cancers), HLA-DR10 (NHL), HLA-DRB (NHL, B cell leukemia), human chorionic gonadotropin (carcinomas), IGF1R (solid tumors, blood cancers), IL-2 receptor (T-cell leukemia and lymphomas), IL-6R (multiple myeloma, RA, Castleman's disease, IL6 dependent tumors), integrins (αvβ3, α5β1, α6β4, α11β3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 (breast, ovarian, cervix, bronchus and gastrointestinal cancer), MUC16 (CA125) (ovarian cancers), CEA (colorectal cancer), gp100 (melanoma), MARTI (melanoma), MPG (melanoma), MS4A1 (membrane-spanning 4-domains subfamily A, small cell lung cancers, NHL), nucleolin, Neu oncogene product (carcinomas), P21 (carcinomas), nectin-4 (carcinomas), paratope of anti-(N-glycolylneuraminic acid, breast, melanoma cancers), PLAP-like testicular alkaline phosphatase (ovarian, testicular cancers), PSMA (prostate tumors), PSA (prostate), ROB04, TAG 72 (tumour associated glycoprotein 72, AML, gastric, colorectal, ovarian cancers), T cell transmembrane protein (cancers), Tie (CD202b), tissue factor, TNFRSF10B (tumor necrosis factor receptor superfamily member 10B, carcinomas), TNFRSF13B (tumor necrosis factor receptor superfamily member 13B, multiple myeloma, NHL, other cancers, RA and SLE), TPBG (trophoblast glycoprotein, renal cell carcinoma), TRAIL-R1 (tumor necrosis apoptosis inducing ligand receptor 1, lymphoma, NHL, colorectal, lung cancers), VCAM-1 (CD106, Melanoma), VEGF, VEGF-A, VEGF-2 (CD309) (various cancers). Some other tumor associated antigen targets have been reviewed (Gerber, et al, mAbs 2009 1:247-253; Novellino et al, Cancer Immunol Immunother. 2005 54:187-207, Franke, et al, Cancer Biother Radiopharm. 2000, 15:459-76, Guo, et al., Adv Cancer Res. 2013; 119: 421-475, Parmiani et al. J Immunol. 2007 178:1975-9). Examples of these antigens include Cluster of Differentiations (CD4, CD5, CD6, CD7, CD8, CD9, CD10, CD11a, CD11b, CD11c, CD12w, CD14, CD15, CD16, CDw17, CD18, CD21, CD23, CD24, CD25, CD26, CD27, CD28, CD29, CD31, CD32, CD34, CD35, CD36, CD37, CD41, CD42, CD43, CD44, CD45, CD46, CD47, CD48, CD49b, CD49c, CD53, CD54, CD55, CD58, CD59, CD61, CD62E, CD62L, CD62P, CD63, CD68, CD69, CD71, CD72, CD79, CD81, CD82, CD83, CD86, CD87, CD88, CD89, CD90, CD91, CD95, CD96, CD100, CD103, CD105, CD106, CD109, CD117, CD120, CD127, CD133, CD134, CD135, CD138, CD141, CD142, CD143, CD144, CD147, CD151, CD152, CD154, CD156, CD158, CD163, CD166, CD168, CD184, CDw186, CD195, CD202 (a, b), CD209, CD235a, CD271, CD303, CD304), annexin A1, nucleolin, endoglin (CD105), ROB04, amino-peptidase N, -like-4 (DLL4), VEGFR-2 (CD309), CXCR4 (CD184), Tie2, B7-H3, WT1, MUC1, LMP2, HPV E6 E7, EGFRvIII, HER-2/neu, idiotype, MAGE A3, p53 nonmutant, NY-ESO-1, GD2, CEA, MelanA/MART1, Ras mutant, gp100, p53 mutant, proteinase3 (PR1), bcr-abl, tyrosinase, survivin, hTERT, sarcoma translocation breakpoints, EphA2, PAP, ML-IAP, AFP, EpCAM, ERG (TMPRSS2 ETS fusion gene), NA17, PAX3, ALK, androgen receptor, cyclin B 1, polysialic acid, MYCN, RhoC, TRP-2, GD3, fucosyl GM1, mesothelin, PSCA, MAGE A1, sLe(a), CYNIC I, PLAC1, GM3, BORIS, Tn, GloboH, ETV6-AML, NY-BR-1, RGSS, SART3, STn, carbonic anhydrase IX, PAXS, OY-TES1, sperm protein 17, LCK, HMWMAA, AKAP-4, SSX2, XAGE 1, B7H3, legumain, Tie 2, Page4, VEGFR2, MAD-CT-1, FAP, PDGFR-β, MAD-CT-2, and Fos-related antigen 1.

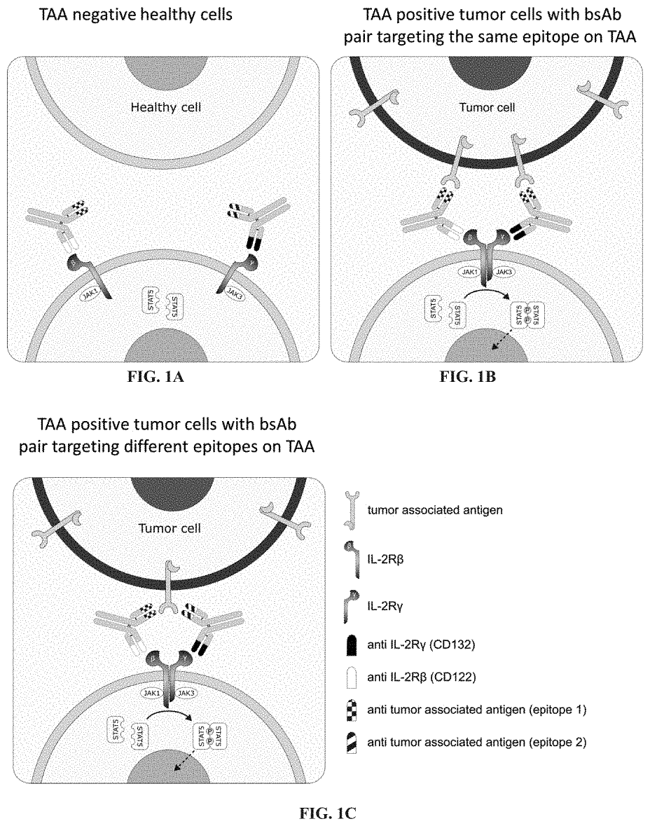

The two bsAbs can target the same epitope (FIG. 1B) or two different epitopes (FIG. 1C) on a given cell surface protein or TAA. In the absence of TAA (i.e., in healthy tissues), the bsAb pair cannot bring the cytokine receptor subunits into proximity and does not induce cytokine activation (FIG. 1A). The invention further provides the targeting of two different cell surface proteins co-expressed on the same cells and in particular two TAAs co-expressed on the surface of tumor cells.

In specific embodiments the invention provides selective IL-2 signaling activation on a specific type of cells using the combination of two bsAbs. One bsAb has one antigen binding domain that binds to IL-2Rβ and a second antigen binding domain that binds to a cell surface protein expressed on the same cell expressing the IL-2Rβ (e.g., T cells or NK cells). The second bsAb has one antigen binding domain that binds to IL-2Rγ and a second antigen domain that binds the same cell surface protein targeted by the first bsAb. The cell surface protein can be specific to a subset of T cells, NK cells or other IL-2R expressing cells. Binding to the same cell surface protein brings the two bsAbs into proximity to each other. The IL-2Rβ and IL-2Rγ binding arms can then bind to and bring together the two receptors and induce signaling activation in the cells expressing both the IL-2Rs and the cell surface protein.

As the two IL-2 receptors, IL-2Rβ (CD122) and IL-2Rγ (CD132), are shared by both IL-2 and IL-15 for signaling, the composition of the invention can be used to selectively activate both IL-2 signaling and IL-15 signaling pathways in either the TME or on a specific subset of cells.

Bispecific Antibodies

The bispecific antibodies (bsAbs) according to the invention may be generated de novo or may be engineered from existing monospecific anti-cytokine receptor subunit antibodies and cell surface antigen antibodies.

The bsAbs of the invention can be based on any of the different antibody formats that have been previously described. In addition, the two bsAb antibodies forming the pair of the invention can be based on the same format or of two different formats. In general, IgG-like formats are preferred as they provide favorable properties such as long half-life and potentially reduced immunogenicity, but any other molecular bispecific format can also be used for the invention.

The heavy and light chain amino acid sequences of the antibodies identified by their United States Adopted Names (USAN are available for example via the American Medical Association at http://_www_ama-assn org or via the CAS registry.

Monospecific anti-cytokine receptor subunit and cell surface antigen binding variable domains may be selected de novo from for example a phage display library, where the phage is engineered to express human immunoglobulins or portions thereof such as Fabs, single chain variable fragments (scFv), or unpaired or paired antibody variable regions and subsequently engineered into a bispecific format. The monospecific anti-cytokine receptor subunit and cell surface antigen variable domains can be isolated for example from phage display libraries expressing antibody heavy and light chain variable regions as fusion proteins with bacteriophage pIX coat protein

The antibody libraries are screened for binding to human cytokine receptor subunit or cell surface antigen extracellular domains and the obtained positive clones are further characterized and the Fabs isolated from the clone lysates. Such phage display methods for isolating human antibodies are established in the art. See for example: U.S. Pat. Nos. 5,223,409; 5,403,484; and 5,571,698, 5,427,908, 5,580,717, 5,969,108, 6,172,197, 5,885,793; 6,521,404; 6,544,731; 6,555,313; 6,582,915 and 6,593,081. The obtained de novo variable regions binding are engineered to bispecific formats using the methods know in the art and described herein.

Antibodies of the present invention have two or more antigen binding sites and are bispecific. Bispecific antibodies of the invention include antibodies having a full length antibody structure. Alternatively, the bispecific antibodies of the invention include antibodies that are less than full length, but contain the antigen binding domains, such as Fab′ fragments.

“Full length antibody” as used herein refers to an antibody having two full length antibody heavy chains and two full length antibody light chains. A full length antibody heavy chain (HC) consists of well known heavy chain variable and constant domains VH, CH1, CH2, and CH3. A full length antibody light chain (LC) consists of well known light chain variable and constant domains VL and CL. The full length antibody may be lacking the C-terminal lysine (K) in either one or both heavy chains.

The term “Fab-arm” or “half molecule” refers to one heavy chain-light chain pair that specifically binds an antigen.

Full length bispecific antibodies of the invention may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. U52010/0015133; US Pat. Publ. No. US2009/0182127; US Pat. Publ. No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849

Correct heavy and light chain pairings can be achieved using immunoglobulin domain crossover as a generic approach for the production of bispecific IgG antibodies as described in WO2015150447A1.

In addition to methods described above, bispecific antibodies of the invention can be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange.

Additionally, bispecific antibodies of the invention can be made using the techniques, including those disclosed in WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions.

However, it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

Exemplary anti-IL-2β antibodies that may be used to engineer bispecific antibodies include for example P2C4 or a variant of P2C4, P2H7 or a variant of P2H7, P2D12 or a variant of P2D12, P1G11 or a variant of P1G11 (as described in WO 2017/021540 A1 and incorporated herein by reference in its entirety).

P2H7 antibody
CDRH1-
(SEQ ID NO: 155)
TYAMH
CDRH2-
(SEQ ID NO: 156)
WINTGNGNTKYSQNFQG
CDRH3-
(SEQ ID NO: 157)
DLGQLERLYFW
CDRL1-
(SEQ ID NO: 158)
RAGQAISSWLA
CDRL2-
(SEQ ID NO: 159)
KASNLES
CDRL3-
(SEQ ID NO: 160)
QQYQSYPYT
VH-
SEQ ID NO: 161)
EVQLVQSGTEVKKPGASVKVSCKASGYTFTTYAMHWVRQ
APGQSLEWMGWINTGNGNTKYSQNFQGRVTMTRDTSIST
AYMELSRLRSDDTAVYYCARDLGQLERLYFWGQGTLVTV
SS
VL-
(SEQ ID NO: 162)
DIQMTQSPSTLSASVGDRVTLSCRAGQAISSWLAWYQQK
PGKAPKLLIYKASNLESGVPSRFSGGGSGAEFTLTISSL
QPDDFATYYCQQYQSYPYTFGQGTKLEIR
P2D12 antibody
CDRH1-
(SEQ ID NO: 163)
SYAMS
CDRH2-
(SEQ ID NO: 164)
AISGSGGSTYYADSVKG
CDRH3-
(SEQ ID NO: 165)
DLGDY
CDRL1-
(SEQ ID NO: 166)
QASQDIGNYLN
CDRL2-
(SEQ ID NO: 167)
DASNLET
CDRL3-
(SEQ ID NO: 168)
LQLYDYPLT
VH-
(SEQ ID NO: 169)
HVQLVETGGGLVQPGGSLRLSCAASGFTFSSYAMSWVRQ
APGKGLEWVSAISGSGGSTYYADSVKGRFTISRDNSKNT
LYLQMNSLRAEDTAVYYCARDLGDYWGQGTLVTVSS
VL-
(SEQ ID NO: 170)
DIQLTQSPSSLSASVGDRVTITCQASQDIGNYLNWYQLK
PGKAPKLLIYDASNLETGVPSRFSGSGSGTDFTFTISSL
QPEDIATYYCLQLYDYPLTFGGGTKVEIK
P1G11 antibody
CDRH1-
(SEQ ID NO: 19)
GYYWS
CDRH2-
(SEQ ID NO: 20)
EINHSGSTNYNPSLKS
CDRH3-
(SEQ ID NO: 171)
SSSGDAFD
CDRL1-
(SEQ ID NO: 172)
TRSSGSIASNYVQ
CDRL2-
(SEQ ID NO: 173)
DDNQRPT
CDRL3-
(SEQ ID NO: 174)
QSSHSTAVV
VH-
(SEQ ID NO: 175)
QVQLQQWGAGLLKPSETLSLTCAVYGGSFSGYYWSWI
RQPPGKGLEWIGEINHSGSTNYNPSLKSRVTISVDTS
KNQFSLKLSSVTAADTAVYYCARSSSGDAFDIWGQGT
MVTVSS
VL-
(SEQ ID NO: 176)
NFMLTQPHSVSESPGKTVTISCTRSSGSIASNYVQW
YQQRPGSSPTTVIFDDNQRPTGVPDRFSAAIDTSSS
SASLTISGLTAEDEADYYCQSSHSTAVVFGGGTKLT
VL

Exemplary anti-IL-2Rβ antibodies that may be used to engineer bispecific antibodies include for example Hu-Mikβ1 (National Cancer Institute, Mayo Clinic, University of Chicago Medicine Celiac Disease Center), single domain antibodies IL-2RB_F09, IL-2RB_F17, IL-2RB_F18, IL-2RB_F20 and IL-2RB_F21 (Katherine E. Harris et al., 2021, A bispecific antibody agonist of the IL-2 heterodimeric receptor preferentially promotes in vivo expansion of CD8 and NK cells, Scientific Reports, doi.org/10.1038/s41598-021-90096-8). Exemplary anti-IL-2Rβ antibodies may also include but are not limited to “AL1”, “AL2”, “AL3”, “AL4” and “AL5” as shown in Table 1.

Exemplary anti-IL-2Rγ antibodies that may be used to engineer bispecific antibodies include for example P1A3, P1A3_B3, P1A3_E8, P1A3_E9, P1A3_B4, P1A3_FW2, P2B9 (as described in WO 2017/021540 A1 and incorporated herein by reference in its entirety).

P1A3_B3, P1A3_E8, P1A3_E9, and
P1A3_B4 Antibodies
CDRH1-
(SEQ ID NO: 19)
GYYWS
CDRH2-
(SEQ ID NO: 20)
EINHSGSTNYNPSLKS
CDRH3-
(SEQ ID NO: 21)
SPGGYSGGYFQH
CDRL1-
(SEQ ID NO: 15)
RSSQSLLHSNGYNYLD
CDRL2-
(SEQ ID NO: 16)
LGSNRDS
CDRL3-
(SEQ ID NO: 17)
MQGTHWPWT
P1A3_B3 Antibody
VH-
(SEQ ID NO: 177)
QVQLQQWGAGLLKPSETLSLTCAVYGGSFSGYYWS
WIRQPPGKGLEWIGEINHFGSTNYNPSLKSRATIS
VDTSKNQFSLKLSSVTAADTAVYYCATSPGGYSGG
YFQHWGQGTLVTVSS
VL-
(SEQ ID NO: 178)
DVVMTQSPLSLPVTPGEPASISCRSSQSLLHSNGY
NYLDWYLQKPGQSPQLLIYLGSNRDSGVPDRFSGS
GSGTDFTLKISRVEAEDVGVYYCMQGTHWPWTFGQ
GTKVEIK
P1A3_E8 Antibody
VH-
(SEQ ID NO: 179)
QVQLQQWGAGMLKPSETLSLTCAVYGGSFSGYYWS
WIRQPPGKGLEWIGEINHFGSTNYNPSLKSRATIS
VDTSKNQFSLKLSSVTAADTAVYYCATSPGGYSGG
YFQHWGQGTLVTVSS
VL-
(SEQ ID NO: 180)
DVVMTQSPLSLPVTPGEPASISCRSSQSLLHSNGY
NYLDWYLQKPGQSPQLLIYLGSNRDSGVPDRFSGS
GSGTDFTLKISRVEAEDVGVYYCMQGTHWPWTFGQ
GTKVEIK
P1A3_E9 Antibody
VH-
(SEQ ID NO: 181)
QVQLQQWGAGLLKPSETLSLTCAVYGGSFSGYYWS
WIRQPPGKGLEWIGEINHFGSTNYNPSLKSRATIS
VDTSKNQFSLKLSSVTAADTAVYYCATSPGGYSGG
YFQHWGQGTLVTVSS
VL-
(SEQ ID NO: 182)
DVVMTQSPLSLPVTPGEPASISCRSSQSLLHSNGY
NYLDWYLQKPGQSPQLLIYLGSNRDSGVPDRFSGS
GSGTDFTLKISRVEAEDVGVYYCMQGTHWPWTFGQ
GTKVEIK
P1A3_B4 Antibody
VH-
(SEQ ID NO: 183)
QVQLQQWGAGLLKPSETLSLTCAVYGGSFSGYYWS
WIRQPPGKGLEWIGEINHFGSTNYNPSLKSRATIS
VDTSKNQFSLKLSSVTAADTAVYYCATSPGGYSGG
YFQHWGQGTLVTVSS
VL-
(SEQ ID NO: 184)
DVVMTQSPLSLPVTPGESVSISCRSSQSLLHSNGY
NYLDWYLQKPGQSPQLLIYLGSNRDSGVPDRFSGS
GSGTDFTLKISRVEAEDVGVYYCMQGTHWPWTFGQ
GTKVEIK

Exemplary anti-IL-2Rγ antibodies may also include but are not limited to single domain antibodies IL-2RG_F05, IL-2RG_F16, IL-2RG_F18, IL-2RG_F19 and IL-2RG_F20 (Katherine E. Harris et al., 2021, A bispecific antibody agonist of the IL-2 heterodimeric receptor preferentially promotes in vivo expansion of CD8 and NK cells, Scientific Reports, doi.org/10.1038/s41598-021-90096-8). Exemplary anti-IL-2Rγ antibodies may also include but are not limited to “AM1”, “AM2”, “AM3”, “AM4”, “AM5”, “AM6”, “AM7”, “AM8”, “AM9”, “AM10” and “AM11”, as shown in Table 1.

Exemplary anti-cell surface (e.g. TAA) antibodies that may be used to engineer bispecific molecules include for example anti-tumor associate antigen (TAA) antibodies known in the art, such as Pertuzumab and Trastuzumab (HER-2); Cetuximab, Necitumumab, Panitumumab and Amivantamab (EGFR); Labetuzumab and Cibisatamab (CEA); Amatuximab (mesothelin); Cordrituzumab (glypican 3); Atezolizumab, Avelumab and Durvalumab (PD-L1); Blinatumomab (CD19); Brentuximab (CD30); Daratumumab (CD38); Gemtuzumab (CD33); Tositumomab 9CD22) or Obinutuzumab, Ocrelizumab, Ofatumumab, Rituximab, and Ibritumomab (CD20).

Additionally, antigen binding domains of the invention may include various other tumor-specific antibody domains known in the art. The antibodies and their respective targets for treatment of cancer include but are not limited to nivolumab (anti-PD-1 Ab), TA99 (anti-gp75), 3F8 (anti-GD2), 8H9 (anti-B7-H3), abagovomab (anti-CA-125 (imitation)), adecatumumab (anti-EpCAM), afutuzumab (anti-CD20), alacizumab pegol (anti-VEGFR2), altumomab pentetate (anti-CEA), amatuximab (anti-mesothelin), AME-133 (anti-CD20), anatumomab mafenatox (anti-TAG-72), apolizumab (anti-HLA-DR), arcitumomab (anti-CEA), bavituximab (anti-phosphatidylserine), bectumomab (anti-CD22), belimumab (anti-BAFF), besilesomab (anti-CEA-related antigen), bevacizumab (anti-VEGF-A), bivatuzumab mertansine (anti-CD44 v6), blinatumomab (anti-CD19), BMS-663513 (anti-CD137), brentuximab vedotin (anti-CD30 (TNFRSF8)), cantuzumab mertansine (anti-mucin CanAg), cantuzumab ravtansine (anti-MUC1), capromab pendetide (anti-prostatic carcinoma cells), carlumab (anti-MCP-1), catumaxomab (anti-EpCAM, CD3), cBR96-doxorubicin immunoconjugate (anti-Lewis-Y antigen), CC49 (anti-TAG-72), cedelizumab (anti-CD4), Ch.14.18 (anti-GD2), ch-TNT (anti-DNA associated antigens), citatuzumab bogatox (anti-EpCAM), cixutumumab (anti-IGF-1 receptor), clivatuzumab tetraxetan (anti-MUC1), conatumumab (anti-TRAIL-R2), CP-870893 (anti-CD40), dacetuzumab (anti-CD40), daclizumab (anti-CD25), dalotuzumab (anti-insulin-like growth factor I receptor), daratumumab (anti-CD38 (cyclic ADP ribose hydrolase)), demcizumab (anti-DLL4), detumomab (anti-B-lymphoma cell), drozitumab (anti-DR5), duligotumab (anti-HER3), dusigitumab (anti-ILGF2), ecromeximab (anti-GD3 ganglioside), edrecolomab (anti-EpCAM), elotuzumab (anti-SLAMF7), elsilimomab (anti-IL-6), enavatuzumab (anti-TWEAK receptor), enoticumab (anti-DLL4), ensituximab (anti-SAC), epitumomab cituxetan (anti-episialin), epratuzumab (anti-CD22), ertumaxomab (anti-HER2/neu, CD3), etaracizumab (anti-integrin αvβ3), faralimomab (anti-Interferon receptor), farletuzumab (anti-folate receptor 1), FBTA05 (anti-CD20), ficlatuzumab (anti-HGF), figitumumab (anti-IGF-1 receptor), flanvotumab (anti-TYRP1(glycoprotein 75)), fresolimumab (anti-TGF (3), futuximab (anti-EGFR), galiximab (anti-CD80), ganitumab (anti-IGF-I), gemtuzumab ozogamicin (anti-CD33), girentuximab (anti-carbonic anhydrase 9 (CA-IX)), glembatumumab vedotin (anti-GPNMB), guselkumab (anti-IL13), ibalizumab (anti-CD4), ibritumomab tiuxetan (anti-CD20), icrucumab (anti-VEGFR-1), igovomab (anti-CA-125), IMAB362 (anti-CLDN18.2), IMC-CS4 (anti-CSF1R), IMC-TR1 (TGFβRII), imgatuzumab (anti-EGFR), inclacumab (anti-selectin P), indatuximab ravtansine (anti-SDC1), inotuzumab ozogamicin (anti-CD22), intetumumab (anti-CD51), ipilimumab (anti-CD152), iratumumab (anti-CD30 (TNFRSF8)), KM3065 (anti-CD20), KW-0761 (anti-CD194), LY2875358 (anti-MET) labetuzumab (anti-CEA), lambrolizumab (anti-PDCD1), lexatumumab (anti-TRAIL-R2), lintuzumab (anti-CD33), lirilumab (anti-KIR2D), lorvotuzumab mertansine (anti-CD56), lucatumumab (anti-CD40), lumiliximab (anti-CD23 (IgE receptor)), mapatumumab (anti-TRAIL-R1), margetuximab (anti-ch4D5), matuzumab (anti-EGFR), mavrilimumab (anti-GMCSF receptor α-chain), milatuzumab (anti-CD74), minretumomab (anti-TAG-72), mitumomab (anti-GD3 ganglioside), mogamulizumab (anti-CCR4), moxetumomab pasudotox (anti-CD22), nacolomab tafenatox (anti-C242 antigen), naptumomab estafenatox (anti-5T4), narnatumab (anti-RON), necitumumab (anti-EGFR), nesvacumab (anti-angiopoietin 2), nimotuzumab (anti-EGFR), nivolumab (anti-IgG4), nofetumomab merpentan, ocrelizumab (anti-CD20), ocaratuzumab (anti-CD20), olaratumab (anti-PDGF-R α), onartuzumab (anti-c-MET), ontuxizumab (anti-TEM1), oportuzumab monatox (anti-EpCAM), oregovomab (anti-CA-125), otlertuzumab (anti-CD37), pankomab (anti-tumor specific glycosylation of MUC1), parsatuzumab (anti-EGFL7), pascolizumab (anti-IL-4), patritumab (anti-HER3), pemtumomab (anti-MUC1), pertuzumab (anti-HER2/neu), pidilizumab (anti-PD-1), pinatuzumab vedotin (anti-CD22), pintumomab (anti-adenocarcinoma antigen), polatuzumab vedotin (anti-CD79B), pritumumab (anti-vimentin), PRO131921 (anti-CD20), quilizumab (anti-IGHE), racotumomab (anti-N-glycolylneuraminic acid), radretumab (anti-fibronectin extra domain-B), ramucirumab (anti-VEGFR2), rilotumumab (anti-HGF), robatumumab (anti-IGF-1 receptor), roledumab (anti-RHD), rovelizumab (anti-CD11 & CD18), samalizumab (anti-CD200), satumomab pendetide (anti-TAG-72), seribantumab (anti-ERBB3), SGN-CD19A (anti-CD19), SGN-CD33A (anti-CD33), sibrotuzumab (anti-FAP), siltuximab (anti-IL-6), solitomab (anti-EpCAM), sontuzumab (anti-episialin), tabalumab (anti-BAFF), tacatuzumab tetraxetan (anti-alpha-fetoprotein), taplitumomab paptox (anti-CD19), telimomab aritox, tenatumomab (anti-tenascin C), teneliximab (anti-CD40), teprotumumab (anti-CD221), TGN1412 (anti-CD28), ticilimumab (anti-CTLA-4), tigatuzumab (anti-TRAIL-R2), TNX-650 (anti-IL-13), tositumomab (anti-CS20), tovetumab (anti-CD140a), TRBS07 (anti-GD2), tregalizumab (anti-CD4), tremelimumab (anti-CTLA-4), TRU-016 (anti-CD37), tucotuzumab celmoleukin (anti-EpCAM), ublituximab (anti-CD20), urelumab (anti-4-1BB), vantictumab (anti-Frizzled receptor), vapaliximab (anti-AOC3 (VAP-1)), vatelizumab (anti-ITGA2), veltuzumab (anti-CD20), vesencumab (anti-NRP1), visilizumab (anti-CD3), volociximab (anti-integrin α5β1), vorsetuzumab mafodotin (anti-CD70), votumumab (anti-tumor antigen CTAA16.88), zalutumumab (anti-EGFR), zanolimumab (anti-CD4), zatuximab (anti-HER1), ziralimumab (anti-CD147 (basigin)), RG7636 (anti-ETBR), RG7458 (anti-MUC16), RG7599 (anti-NaPi2b), MPDL3280A (anti-PD-L1), RG7450 (anti-STEAP1), and GDC-0199 (anti-Bcl-2).

Exemplary amino acid sequences of antigen binding domains of the bispecific antibodies of the disclosure are shown in Table 1.

TABLE 1
Amino Acid Sequences of Antigen Binding Domains

| Antigen | | | |
|---|---|---|---|
| Binding Arm | | | |
| Name/ | | | Sequence |
| Antigen | Domain | Amino Acid Sequence | Identifier |
| Common | CDR-H1 | GFTFSSYA | SEQ ID NO: 1 |
| heavy chain | | | |
| Common | CDR-H2 | ISGSGGST | SEQ ID NO: 2 |
| heavy chain | | | |
| Common | CDR-H3 | AKSYGAFDY | SEQ ID NO: 3 |
| heavy chain | | | |
| Common | VH | EVQLLESGGGLVQPGGSLRLSCAASGFTFSSYAMSWVRQAPG | SEQ ID NO: 4 |
| heavy chain | | KGLEWVSAISGSGGSTYYADSVKGRFTISRDNSKNTLYLQMN | |
| | | SLRAEDTAVYYCAKSYGAFDYWGQGTLVTVSS | |
| Common | HC | EVQLLESGGGLVQPGGSLRLSCAASGFTFSSYAMSWVRQAPG | SEQ ID NO: 5 |
| heavy chain | (LALA + | KGLEWVSAISGSGGSTYYADSVKGRFTISRDNSKNTLYLQMN | |
| | P329A | SLRAEDTAVYYCAKSYGAFDYWGQGTLVTVSSASTKGPSVFP | |
| | mutation) | LAPSSKSTSGGTAALGCLVKDYFPEPVTVSWNSGALTSGVHT | |
| | | FPAVLQSSGLYSLSSVVTVPSSSLGTQTYICNVNHKPSNTKV | |
| | | DKRVEPKSCDKTHTCPPCPAPEAAGGPSVFLFPPKPKDTLMI | |
| | | SRTPEVTCVVVDVSHEDPEVKFNWYVDGVEVHNAKTKPREEQ | |
| | | YNSTYRVVSVLTVLHQDWLNGKEYKCKVSNKALAAPIEKTIS | |
| | | KAKGQPREPQVYTLPPSREEMTKNQVSLTCLVKGFYPSDIAV | |
| | | EWESNGQPENNYKTTPPVLDSDGSFFLYSKLTVDKSRWQQGN | |
| | | VFSCSVMHEALHNHYTQKSLSLSPG | |
| P2C4/IL2Rb | LC (light | QSALTQPASVSGSPGQSIAISCTGTSSDIGHYDFVSWYQQHP | SEQ ID NO: 6 |
| | chain) | GTAPKLIIYDINNRPSGISNRFSGSKSDNMASLTISGLQPED | |
| | | EADYYCSAYTSSDTLVFGGGTKLT | |
| P2C4/IL2Rb | CDR-L1 | TGTSSDIGHYDFVS | SEQ ID NO: 7 |
| P2C4/IL2Rb | CDR-L2 | DINNRPS | SEQ ID NO: 8 |
| P2C4/IL2Rb | CDR-L3 | SAYTSSDTLV | SEQ ID NO: 9 |
| P2C4/IL2Rb | HC | EVQLVQSGAEVKKPGSSVKVSCKASGYTFTNYYMHWVRQAPG | SEQ ID NO: 10 |
| | (heavy | QGLEWMGAIMPSRGGTSYPQKFQGRVTMTGDTSTSTVYMELS | |
| | chain) | SLRSEDTAVYYCARGEYYYDSSGYYYWGQGTLVTVSS | |
| P2C4/IL2Rb | HC- | NYYMH | SEQ ID NO: 11 |
| | CDR1 | | |
| P2C4/IL2Rb | HC- | AIMPSRGGTSYPQKFQG | SEQ ID NO: 12 |
| | CDR2 | | |
| P2C4/IL2Rb | HC- | GEYYYDSSGYYY | SEQ ID NO: 13 |
| | CDR3 | | |
| P1A3/IL2Rg | LC (light | DVVMTQSPLSLPVTPGEPASISCRSSQSLLHSNGYNYLDWYL | SEQ ID NO: 14 |
| | chain) | QKPGQSPQLLIYLGSNRDSGVPDRFSGSGSGTDFTLKISRVE | |
| | | AEDVGVYYCMQGTHWPWTFGQGTKVEIK | |
| P1A3/IL2Rg | CDR-L1 | RSSQSLLHSNGYNYLD | SEQ ID NO: 15 |
| P1A3/IL2Rg | CDR-L2 | LGSNRDS | SEQ ID NO: 16 |
| P1A3/IL2Rg | CDR-L3 | MQGTHWPWT | SEQ ID NO: 17 |
| P1A3/IL2Rg | HC | QVQLQQWGAGLLKPSETLSLTCAVYGGSFSGYYWSWIRQPPG | SEQ ID NO: 18 |
| | (heavy | KGLEWIGEINHSGSTNYNPSLKSRATISVDTSKNQFSLKLSS | |
| | chain) | VTAADTAVYYCATSPGGYSGGYFQHWGQGTLVTVSS | |
| P1A3/IL2Rg | HC- | GYYWS | SEQ ID NO: 19 |
| | CDR1 | | |
| P1A3/IL2Rg | HC- | EINHSGSTNYNPSLKS | SEQ ID NO: 20 |
| | CDR2 | | |
| P1A3/IL2Rg | HC- | SPGGYSGGYFQH | SEQ ID NO: 21 |
| | CDR3 | | |
| Trastuzumab | LC (light | FGGGTKLTVLSSASTKGPSVFPLAPSSKSTSGGTAALGCLVK | SEQ ID NO: 22 |
| /HER2 | chain)- | DYFPEPVTVSWNSGALTSGVHTFPAVLQSSGLYSLSSVVTVP | |
| | CrossMab | SSSLGTQTYICNVNHKPSNTKVDKKVEPKSCDKTHTCPPCPA | |
| | format | PEAAGGPSVFLFPPKPKDTLMISRTPEVTCVVVDVSHEDPEV | |
| | | KFNWYVDGVEVHNAKTKPREEQYNSTYRVVSVLTVLHQDWLN | |
| | | GKEYKCKVSNKALAAPIEKTISKAKGQPREPQVYTLPPCRDE | |
| | | LTKNQVSLWCLVKGFYPSDIAVEWQSNGQTENNYKTTPPVLD | |
| | | SDGSFFLYSKLTVDKSRWQQGNVFSCSVMHEALHNHYTQKSL | |
| | | SLSPG | |
| Trastuzumab | CDR-L1 | RASQDVNTAVA | SEQ ID NO: 23 |
| /HER2 | | | |
| Trastuzumab | CDR-L2 | SASFLY | SEQ ID NO: 24 |
| /HER2 | | | |
| Trastuzumab | CDR-L3 | QQHYTTPPT | SEQ ID NO: 25 |
| /HER2 | | | |
| Trastuzumab | HC | WGQGTLVTVSSVAAPSVFIFPPSDEQLKSGTASVVCLLNNFY | SEQ ID NO: 26 |
| /HER2 | (heavy | PREAKVQWKVDNALQSGNSQESVTEQDSKDSTYSLSSTLTLS | |
| | chain)- | KADYEKHKVYACEVTHQGLSSPVTKSFNRGEC | |
| | CrossMab | | |
| | format | | |
| Trastuzumab | HC- | GFNIKDTYIH | SEQ ID NO: 27 |
| /HER2 | CDR1 | | |
| Trastuzumab | HC- | ARIYPTNGYTRYADSVKG | SEQ ID NO: 28 |
| /HER2 | CDR2 | | |
| Trastuzumab | HC- | SRWGGDGFYAMDY | SEQ ID NO: 29 |
| /HER2 | CDR3 | | |
| Pertuzumab | LC (light | FGGGTKLTVLSSASTKGPSVFPLAPSSKSTSGGTAALGCLVK | SEQ ID NO: 30 |
| /HER2 | chain)- | DYFPEPVTVSWNSGALTSGVHTFPAVLQSSGLYSLSSVVTVP | |
| | CrossMab | SSSLGTQTYICNVNHKPSNTKVDKKVEPKSCDKTHTCPPCPA | |
| | format | PEAAGGPSVFLFPPKPKDTLMISRTPEVTCVVVDVSHEDPEV | |
| | | KFNWYVDGVEVHNAKTKPREEQYNSTYRVVSVLTVLHQDWLN | |
| | | GKEYKCKVSNKALAAPIEKTISKAKGQPREPQVYTLPPCRDE | |
| | | LTKNQVSLWCLVKGFYPSDIAVEWQSNGQTENNYKTTPPVLD | |
| | | SDGSFFLYSKLTVDKSRWQQGNVFSCSVMHEALHNHYTQKSL | |
| | | SLSPG | |
| Pertuzumab | CDR-L1 | KASQDVSIGVA | SEQ ID NO: 31 |
| /HER2 | | | |
| Pertuzumab | CDR-L2 | SASYRY | SEQ ID NO: 32 |
| /HER2 | | | |
| Pertuzumab | CDR-L3 | QQYYIYPYT | SEQ ID NO: 33 |
| /HER2 | | | |
| Pertuzumab | HC | WGQGTLVTVSSVAAPSVFIFPPSDEQLKSGTASVVCLLNNFY | SEQ ID NO: 34 |
| /HER2 | (heavy | PREAKVQWKVDNALQSGNSQESVTEQDSKDSTYSLSSTLTLS | |
| | chain)- | KADYEKHKVYACEVTHQGLSSPVTKSFNRGEC | |
| | CrossMab | | |
| | format | | |
| Pertuzumab | HC- | GFTFTDYTMD | SEQ ID NO: 35 |
| /HER2 | CDR1 | | |
| Pertuzumab | HC- | ADVNPNSGGSIYNQRFKG | SEQ ID NO: 36 |
| /HER2 | CDR2 | | |
| Pertuzumab | HC- | GPSFYFDY | SEQ ID NO: 37 |
| /HER2 | CDR3 | | |
| AL1/IL2Rb | LC (light | DIQMTQSPSSLSASVGDRVTITCQASQDISNYLNWYQQKPGK | SEQ ID NO: 38 |
| | chain) | APKLLIYDASNLETGVPSRFSGSGSGTDFTFTISSLQPEDIA | |
| | | TYYCQQDANDPRTFGQGTKVEIKRTVAAPSVFIFPPSDEQLK | |
| | | SGTASVVCLLNNFYPREAKVQWKVDNALQSGNSQESVTEQDS | |
| | | KDSTYSLSSTLTLSKADYEKHKVYACEVTHQGLSSPVTKSFN | |
| | | RGEC | |
| AL1/IL2Rb | CDR-L1 | QDISNY | SEQ ID NO: 39 |
| AL1/IL2Rb | CDR-L2 | DAS | SEQ ID NO: 40 |
| AL1/IL2Rb | CDR-L3 | QQDANDPRT | SEQ ID NO: 41 |
| AL1/IL2Rb | VL (light | DIQMTQSPSSLSASVGDRVTITCQASQDISNYLNWYQQKPGK | SEQ ID NO: 42 |
| | chain | APKLLIYDASNLETGVPSRFSGSGSGTDFTFTISSLQPEDIA | |
| | variable | TYYCQQDANDPRTFGQGTKVEIK | |
| | region) | | |
| AL2/IL2Rb | LC (light | DIQMTQSPSSLSASVGDRVTITCQASQDIGTYLNWYQQKPGK | SEQ ID NO: 43 |
| | chain) | APKLLIYEASTLETGVPSRFSGSGSGTDFTFTISSLQPEDIA | |
| | | TYYCQQDGARDDYATFGQGTKVEIKRTVAAPSVFIFPPSDEQ | |
| | | LKSGTASVVCLLNNFYPREAKVQWKVDNALQSGNSQESVTEQ | |
| | | DSKDSTYSLSSTLTLSKADYEKHKVYACEVTHQGLSSPVTKS | |
| | | FNRGEC | |
| AL2/IL2Rb | CDR-L1 | QDIGTY | SEQ ID NO: 44 |
| AL2/IL2Rb | CDR-L2 | EAS | SEQ ID NO: 45 |
| AL2/IL2Rb | CDR-L3 | QQDGARDDYAT | SEQ ID NO: 46 |
| AL2/IL2Rb | VL (light | DIQMTQSPSSLSASVGDRVTITCQASQDIGTYLNWYQQKPGK | SEQ ID NO: 47 |
| | chain | APKLLIYEASTLETGVPSRFSGSGSGTDFTFTISSLQPEDIA | |
| | variable | TYYCQQDGARDDYATFGQGTKVEIK | |
| | region) | | |
| AL3/IL2Rb | LC (light | DIQMTQSPSSLSASVGDRVTITCQASQDIDDYLNWYQQKPGK | SEQ ID NO: 48 |
| | chain) | APKLLIYDASNLQSGVPSRFSGSGSGTDFTLTISSLQPEDFA | |
| | | TYYCQQDSMDPRTFGQGTKVEIKRTVAAPSVFIFPPSDEQLK | |
| | | SGTASVVCLLNNFYPREAKVQWKVDNALQSGNSQESVTEQDS | |
| | | KDSTYSLSSTLT | |
| | | LSKADYEKHKVYACEVTHQGLSSPVTKSFNRGEC | |
| AL3/IL2Rb | CDR-L1 | QDIDDY | SEQ ID NO: 49 |
| AL3/IL2Rb | CDR-L2 | DAS | SEQ ID NO: 50 |
| AL3/IL2Rb | CDR-L3 | QQDSMDPRT | SEQ ID NO: 51 |
| AL3/IL2Rb | VL (light | DIQMTQSPSSLSASVGDRVTITCQASQDIDDYLNWYQQKPGK | SEQ ID NO: 52 |
| | chain | APKLLIYDASNLQSGVPSRFSGSGSGTDFTLTISSLQPEDFA | |
| | variable | TYYCQQDSMDPRTFGQGTKVEIK | |
| | region) | | |
| AL4/IL2Rb | LC (light | DIQMTQSPSSLSASVGDRVTITCRASQSIDEYLNWYQQKPGK | SEQ ID NO: 53 |
| | chain) | APKLLIYEASKLQSGVPSRFSGSGSGTDFTLTISSLQPEDFA | |
| | | TYYCQQDGAMDTYATFGQGTKVEIKRTVAAPSVFIFPPSDEQ | |
| | | LKSGTASVVCLLNNFYPREAKVQWKVDNALQSGNSQESVTEQ | |
| | | DSKDSTYSLSST | |
| | | LTLSKADYEKHKVYACEVTHQGLSSPVTKSFNRGEC | |
| AL4/IL2Rb | CDR-L1 | QSIDEY | SEQ ID NO: 54 |
| AL4/IL2Rb | CDR-L2 | EAS | SEQ ID NO: 55 |
| AL4/IL2Rb | CDR-L3 | QQDGAMDTYAT | SEQ ID NO: 56 |
| AL4/IL2Rb | VL (light | DIQMTQSPSSLSASVGDRVTITCRASQSIDEYLNWYQQKPGK | SEQ ID NO: 57 |
| | chain | APKLLIYEASKLQSGVPSRFSGSGSGTDFTLTISSLQPEDFA | |
| | variable | TYYCQQDGAMDTYATFGQGTKVEIK | |
| | region) | | |
| AL5/IL2Rb | LC (light | EIVLTQSPATLSLSPGERATLSCRASQSVDEYLAWYQQKPGQ | SEQ ID NO: 58 |
| | chain) | APRLLIYDASERATGIPARFSGSGSGTDFTLTISSLEPEDFA | |
| | | VYYCQQDATDPRTFGQGTKVEIKRTVAAPSVFIFPPSDEQLK | |
| | | SGTASVVCLLNNFYPREAKVQWKVDNALQSGNSQESVTEQDS | |
| | | KDSTYSLSSTLT | |
| | | LSKADYEKHKVYACEVTHQGLSSPVTKSFNRGEC | |
| AL5/IL2Rb | CDR-L1 | QSVDEY | SEQ ID NO: 59 |
| AL5/IL2Rb | CDR-L2 | DAS | SEQ ID NO: 60 |
| AL5/IL2Rb | CDR-L3 | QQDATDPRT | SEQ ID NO: 61 |
| AL5/IL2Rb | VL (light | EIVLTQSPATLSLSPGERATLSCRASQSVDEYLAWYQQKPGQ | SEQ ID NO: 62 |
| | chain | APRLLIYDASERATGIPARFSGSGSGTDFTLTISSLEPEDFA | |
| | variable | VYYCQQDATDPRTFGQGTKVEIK | |
| | region) | | |
| AM1/IL2Rg | LC (light | DIQMTQSPSSLSASVGDRVTITCRASQSTYYYLNWYQQKPGK | SEQ ID NO: 63 |
| | chain) | APKLLIYDASALQSGVPSRFSGSGSGTDFTLTISSLQPEDFA | |
| | | TYYCQQIDFTAGSITFGQGTKVEIKRTVAAPSVFIFPPSDEQ | |
| | | LKSGTASVVCLLNNFYPREAKVQWKVDNALQSGNSQESVTEQ | |
| | | DSKDSTYSLSST | |
| | | LTLSKADYEKHKVYACEVTHQGLSSPVTKSFNRGEC | |
| AM1/IL2Rg | CDR-L1 | QSIYYY | SEQ ID NO: 64 |
| AM1/IL2Rg | CDR-L2 | DAS | SEQ ID NO: 65 |
| AM1/IL2Rg | CDR-L3 | QQIDFTAGSIT | SEQ ID NO: 66 |
| AM1/IL2Rg | VL (light | DIQMTQSPSSLSASVGDRVTITCRASQSIYYYLNWYQQKPGK | SEQ ID NO: 67 |
| | chain | APKLLIYDASALQSGVPSRFSGSGSGTDFTLTISSLQPEDFA | |
| | variable | TYYCQQIDFTAGSITFGQGTKVEIK | |
| | region) | | |
| AM2/IL2Rg | LC (light | DIQLTQSPSFLSASVGDRVTITCRASQTIDAPLRWYQQKPGK | SEQ ID NO: 68 |
| | chain) | APKLLIYLTSSLQSGVPSRFSGSGSGTEFTLTISSLQPEDFA | |
| | | TYYCQQGYAAGPSTFGQGTKVEIKRTVAAPSVFIFPPSDEQL | |
| | | KSGTASVVCLLNNFYPREAKVQWKVDNALQSGNSQESVTEQD | |
| | | SKDSTYSLSSTLTLSKADYEKHKVYACEVTHQGLSSPVTKSF | |
| | | NRGEC | |
| AM2/IL2Rg | CDR-L1 | QTIDAP | SEQ ID NO: 69 |
| AM2/IL2Rg | CDR-L2 | LTS | SEQ ID NO: 70 |
| AM2/IL2Rg | CDR-L3 | QQGYAAGPST | SEQ ID NO: 71 |
| AM2/IL2Rg | VL (light | DIQLTQSPSFLSASVGDRVTITCRASQTIDAPLRWYQQKPGK | SEQ ID NO: 72 |
| | chain | APKLLIYLTSSLQSGVPSRFSGSGSGTEFTLTISSLQPEDFA | |
| | variable | TYYCQQGYAAGPSTFGQGTKVEIK | |
| | region) | | |
| AM3/IL2Rg | LC (light | DIQMTQSPSTLSASVGDTVTITCRASHYITTWLAWYQQKPGK | SEQ ID NO: 73 |
| | chain) | APKLLIYDVSSLESGVPSRFRGRGSGTEFTLTISSLQPDDFA | |
| | | TYYCQQYESYSPTFGQGTKVEIKRTVAAPSVFIFPPSDEQLK | |
| | | SGTASVVCLLNNFYPREAKVQWKVDNALQSGNSQESVTEQDS | |
| | | KDSTYSLSSTLTLSKADYEKHKVYACEVTHQGLSSPVTKSFN | |
| | | RGEC | |
| AM3/IL2Rg | CDR-L1 | HYITTW | SEQ ID NO: 74 |
| AM3/IL2Rg | CDR-L2 | DVS | SEQ ID NO: 75 |
| AM3/IL2Rg | CDR-L3 | QQYESYSPT | SEQ ID NO: 76 |
| AM3/IL2Rg | VL (light | DIQMTQSPSTLSASVGDTVTITCRASHYITTWLAWYQQKPGK | SEQ ID NO: 77 |
| | chain | APKLLIYDVSSLESGVPSRFRGRGSGTEFTLTISSLQPDDFA | |
| | variable | TYYCQQYESYSPTFGQGTKVEIK | |
| | region) | | |
| AM4/IL2Rg | LC (light | DIQMTQSPSTLSASVGDRVTITCRASQTIYGPLNWYQQKPGK | SEQ ID NO: 78 |
| | chain) | APKLLIYSTSYLESGVPSRFSGSGSGTEFTLTISSLQPDDFA | |
| | | TYYCQQAGYASAPTFGQGTKVEIKRTVAAPSVFIFPPSDEQL | |
| | | KSGTASVVCLLNNFYPREAKVQWKVDNALQSGNSQESVTEQD | |
| | | SKDSTYSLSSTLTLSKADYEKHKVYACEVTHQGLSSPVTKSF | |
| | | NRGEC | |
| AM4/IL2Rg | CDR-L1 | QTIYGP | SEQ ID NO: 79 |
| AM4/IL2Rg | CDR-L2 | STS | SEQ ID NO: 80 |
| AM4/IL2Rg | CDR-L3 | QQAGYASAPT | SEQ ID NO: 81 |
| AM4/IL2Rg | VL (light | DIQMTQSPSTLSASVGDRVTITCRASQTIYGPLNWYQQKPGK | SEQ ID NO: 82 |
| | chain | APKLLIYSTSYLESGVPSRFSGSGSGTEFTLTISSLQPDDFA | |
| | variable | TYYCQQAGYASAPTFGQGTKVEIK | |
| | region) | | |
| AM5/IL2Rg | LC (light | DIVMTQSPDSLAVSLGERATINCKSSQSVLYSEVAYTALAWY | SEQ ID NO: 83 |
| | chain) | QQKPGQPPKLLIYATSTRESGVPDRFSGSGSGTDFTLTISSL | |
| | | QAEDVAVYYCQQGYGHPTFGQGTKVEIKRTVAAPSVFIFPPS | |
| | | DEQLKSGTASVVCLLNNFYPREAKVQWKVDNALQSGNSQESV | |
| | | TEQDSKDSTYSL | |
| | | SSTLTLSKADYEKHKVYACEVTHQGLSSPVTKSFNRGEC | |
| AM5/IL2Rg | CDR-L1 | QSVLYSEVAYTA | SEQ ID NO: 84 |
| AM5/IL2Rg | CDR-L2 | ATS | SEQ ID NO: 85 |
| AM5/IL2Rg | CDR-L3 | QQGYGHPT | SEQ ID NO: 86 |
| AM5/IL2Rg | VL (light | DIVMTQSPDSLAVSLGERATINCKSSQSVLYSEVAYTALAWY | SEQ ID NO: 87 |
| | chain | QQKPGQPPKLLIYATSTRESGVPDRFSGSGSGTDFTLTISSL | |
| | variable | QAEDVAVYYCQQGYGHPTFGQGTKVEIK | |
| | region) | | |
| AM6/IL2Rg | LC (light | DIVMTQSPDSLAVSLGERATINCKSSQSVLYDDFGNANLAWY | SEQ ID NO: 88 |
| | chain) | QQKPGQPPKLLIYYGSYRESGVPDRFSGSGSGTDFTLTISSL | |
| | | QAEDVAVYYCQQVDVGLAITFGQGTKVEIKRTVAAPSVFIFP | |
| | | PSDEQLKSGTASVVCLLNNFYPREAKVQWKVDNALQSGNSQE | |
| | | SVTEQDSKDSTY | |
| | | SLSSTLTLSKADYEKHKVYACEVTHQGLSSPVTKSFNRGEC | |
| AM6/IL2Rg | CDR-L1 | QSVLYDDFGNAN | SEQ ID NO: 89 |
| AM6/IL2Rg | CDR-L2 | YGS | SEQ ID NO: 90 |
| AM6/IL2Rg | CDR-L3 | QQVDVGLAIT | SEQ ID NO: 91 |
| AM6/IL2Rg | VL (light | DIVMTQSPDSLAVSLGERATINCKSSQSVLYDDFGNANLAWY | SEQ ID NO: 92 |
| | chain | QQKPGQPPKLLIYYGSYRESGVPDRFSGSGSGTDFTLTISSL | |
| | variable | QAEDVAVYYCQQVDVGLAITFGQGTKVEIK | |
| | region) | | |
| AM7/IL2Rg | LC (light | DIQLTQSPSFLSASVGDRVTITCRASQDIGIELAWYQQKPGK | SEQ ID NO: 93 |
| | chain) | APKLLIYFESHLQSGVPSRFSGSGSGTEFTLTISSLQPEDFA | |
| | | TYYCQQIRIDPTFGQGTKVEIKRTVAAPSVFIFPPSDEQLKS | |
| | | GTASVVCLLNNFYPREAKVQWKVDNALQSGNSQESVTEQDSK | |
| | | DSTYSLSSTLTL | |
| | | SKADYEKHKVYACEVTHQGLSSPVTKSFNRGEC | |
| AM7/IL2Rg | CDR-L1 | QDIGIE | SEQ ID NO: 94 |
| AM7/IL2Rg | CDR-L2 | FES | SEQ ID NO: 95 |
| AM7/IL2Rg | CDR-L3 | QQIRIDPT | SEQ ID NO: 96 |
| AM7/IL2Rg | VL (light | DIQLTQSPSFLSASVGDRVTITCRASQDIGIELAWYQQKPGK | SEQ ID NO: 97 |
| | chain | APKLLIYFESHLQSGVPSRFSGSGSGTEFTLTISSLQPEDFA | |
| | variable | TYYCQQIRIDPTFGQGTKVEIK | |
| | region) | | |
| AM8/IL2Rg | LC (light | EIVLTQSPGTLSLSPGERATLSCRASQDVATRGLAWYQQKPG | SEQ ID NO: 98 |
| | chain) | QAPRLLIYGASSRATGIPDRFSGSGSGTDFTLTISRLEPEDF | |
| | | AVYYCQQYELEHPATFGQGTKVEIKRTVAAPSVFIFPPSDEQ | |
| | | LKSGTASVVCLLNNFYPREAKVQWKVDNALQSGNSQESVTEQ | |
| | | DSKDSTYSLSST | |
| | | LTLSKADYEKHKVYACEVTHQGLSSPVTKSFNRGEC | |
| AM8/IL2Rg | CDR-L1 | QDVATRG | SEQ ID NO: 99 |
| AM8/IL2Rg | CDR-L2 | GAS | SEQ ID NO: 100 |
| AM8/IL2Rg | CDR-L3 | QQYELEHPAT | SEQ ID NO: 101 |
| AM8/IL2Rg | VL (light | EIVLTQSPGTLSLSPGERATLSCRASQDVATRGLAWYQQKPG | SEQ ID NO: 102 |
| | chain | QAPRLLIYGASSRATGIPDRFSGSGSGTDFTLTISRLEPEDF | |
| | variable | AVYYCQQYELEHPATFGQGTKVEIK | |
| | region) | | |
| AM9/IL2Rg | LC (light | DIQMTQSPSSLSASVGDRVTITCQASQDIAGYLNWYQQKPGK | SEQ ID NO: 103 |
| | chain) | APKLLIYTASTLETGVPSRFSGSGSGTDFTFTISSLQPEDIA | |
| | | TYYCQQWAFGPVTFGQGTKVEIKRTVAAPSVFIFPPSDEQLK | |
| | | SGTASVVCLLNNFYPREAKVQWKVDNALQSGNSQESVTEQDS | |
| | | KDSTYSLSSTLT | |
| | | LSKADYEKHKVYACEVTHQGLSSPVTKSFNRGEC | |
| AM9/IL2Rg | CDR-L1 | QDIAGY | SEQ ID NO: 104 |
| AM9/IL2Rg | CDR-L2 | TAS | SEQ ID NO: 105 |
| AM9/IL2Rg | CDR-L3 | QQWAFGPVT | SEQ ID NO: 106 |
| AM9/IL2Rg | VL (light | DIQMTQSPSSLSASVGDRVTITCQASQDIAGYLNWYQQKPGK | SEQ ID NO: 107 |
| | chain | APKLLIYTASTLETGVPSRFSGSGSGTDFTFTISSLQPEDIA | |
| | variable | TYYCQQWAFGPVTFGQGTKVEIK | |
| | region) | | |
| AM10/IL2Rg | LC (light | EIVLTQSPATLSLSPGERATLSCRASQSVFANLNWYQQKPGQ | SEQ ID NO: 108 |
| | chain) | APRLLIYDSSGRATGIPARFSGSGSGTDFTLTISSLEPEDFA | |
| | | VYYCQQGFGPSLTFGQGTKVEIKRTVAAPSVFIFPPSDEQLK | |
| | | SGTASVVCLLNNFYPREAKVQWKVDNALQSGNSQESVTEQDS | |
| | | KDSTYSLSSTLT | |
| | | LSKADYEKHKVYACEVTHQGLSSPVTKSFNRGEC | |
| AM10/IL2Rg | CDR-L1 | QSVFAN | SEQ ID NO: 109 |
| AM10/IL2Rg | CDR-L2 | DSS | SEQ ID NO: 110 |
| AM10/IL2Rg | CDR-L3 | QQGFGPSLT | SEQ ID NO: 111 |
| AM10/IL2Rg | VL (light | EIVLTQSPATLSLSPGERATLSCRASQSVFANLNWYQQKPGQ | SEQ ID NO: 112 |
| | chain | APRLLIYDSSGRATGIPARFSGSGSGTDFTLTISSLEPEDFA | |
| | variable | VYYCQQGFGPSLTFGQGTKVEIK | |
| | region) | | |
| AM11/IL2Rg | LC (light | EIVLTQSPGTLSLSPGERATLSCRASQNVNHNFLTWYQQKPG | SEQ ID NO: 113 |
| | chain) | QAPRLLIYSASARATGIPDRFSGSGSGTDFTLTISRLEPEDF | |
| | | AVYYCQQFNYAPLTFGQGTKVEIKRTVAAPSVFIFPPSDEQL | |
| | | KSGTASVVCLLNNFYPREAKVQWKVDNALQSGNSQESVTEQD | |
| | | SKDSTYSLSSTL | |
| | | TLSKADYEKHKVYACEVTHQGLSSPVTKSFNRGEC | |
| AM11/IL2Rg | CDR-L1 | QNVNHNF | SEQ ID NO: 114 |
| AM11/IL2Rg | CDR-L2 | SAS | SEQ ID NO: 115 |
| AM11/IL2Rg | CDR-L3 | QQFNYAPLT | SEQ ID NO: 116 |
| AM11/IL2Rg | VL (light | EIVLTQSPGTLSLSPGERATLSCRASQNVNHNFLTWYQQKPG | SEQ ID NO: 117 |
| | chain | QAPRLLIYSASARATGIPDRFSGSGSGTDFTLTISRLEPEDF | |
| | variable | AVYYCQQFNYAPLTFGQGTKVEIK | |
| | region) | | |
| HPN536/ | heavy | EVOLVESGGGLVQPGGSLKLSCAASGNTFNKYAMNWVRQAPG | SEQ ID NO: 118 |
| CD3 binding | chain | KGLEWVARIRSKYNNYATYYADSVKDRFTISRDDSKNTAYLQ | |
| arm | | MNNLKTEDTAVYYCVRHGNFGDSYISYWAYWGQGTLVTVSS | |
| HPN536 | light | QTVVTQEPSLTVSPGGTVTLTCGSSTGAVTHGNYPNWVQQKP | SEQ ID NO: 119 |
| /CD3 binding | chain | GQAPRGLIGGTKVLAPGTPARFSGSLLGGKAALTLSGVQPED | |
| arm | | EAEYYCVLWYSNRWVFGGGTKLTVL | |
| HPN536 | sdAb | QVQLVESGGGVVQAGGSLRLSCAASGSTFSIRAMRWYRQAPG | SEQ ID NO: 120 |
| sdAb/MLSN | | TERDLVAVIYGSSTYYADAVKGRFTISRDNSKNTLYLQMNSL | |
| binding arm | | RAEDTAVYYCNADTIGTARDYWGQGTLVTVSS | |
| H4G5- | heavy | EVQLVQSGAEVKKPGASVKVSCKASGYTFTNYYIHWVRQAPG | SEQ ID NO: 121 |
| Trastuzumab/ | chain | QGLEWIGWIYPGDGNTKYNEKFKGRATLTADTSTSTAYLELS | |
| CD3 binding | variable | SLRSEDTAVYYCARDSYSNYYFDYWGQGTLVTVSS | |
| arm | region | | |
| H4G5- | light | DIVMTQSPDSLAVSLGERATINCKSSQSLLNSRTRKNYLAWY | SEQ ID NO: 122 |
| Trastuzumab/ | chain | QQKPGQPPKLLIYWASTRESGVPDRFSGSGSGTDFTLTISSL | |
| CD3 binding | variable | QAEDVAVYYCTQSFILRTFGQGTKVEIK | |
| arm | region | | |
| H4G5- | heavy | EVOLVESGGGLVQPGGSLRLSCAASGFNIKDTYIHWVRQAPG | SEQ ID NO: 123 |
| Trastuzumab/ | chain | KGLEWVARIYPTNGYTRYADSVKGRFTISADTSKNTAYLQMN | |
| HER2 | variable | SLRAEDTAVYYCSRWGGDGFYAMDYWGQGTLVTVSS | |
| binding arm | region | | |
| H4G5- | light | DIQMTQSPSSLSASVGDRVTITCRASQDVNTAVAWYQQKPGK | SEQ ID NO: 124 |
| Trastuzumab/ | chain | APKLLIYSASFLYSGVPSRFSGSRSGTDFTLTISSLQPEDFA | |
| HER2 | variable | TYYCQQHYTTPPTFGGGTKLTVLSS | |
| binding arm | region | | |
| O30/MSLN | LC (light | QSVLTQPPSASGTPGQRVTISCSGSSSNIAHGPVNWYQQLPG | SEQ ID NO: 125 |
| | chain) | TAPKLLIYATNHRPSGVPDRFSGSKSGTTASLTISGLQSEDE | |
| | | ADYYCAAYDLTGWFAYAVFGGGTKLTVLGQPKAAPSVTLFPP | |
| | | SSEELQANKATLVCLISDFYPGAVTVAWKADSSPVKAGVETT | |
| | | TPSKQSNNKYAASSYLSLTPEQWKSHRSYSCQVTHEGSTVEK | |
| | | TVAPTECS | |
| O30/MSLN | CDR-L1 | SSNIAHGP | SEQ ID NO: 126 |
| O30/MSLN | CDR-L2 | ATN | SEQ ID NO: 127 |
| O30/MSLN | CDR-L3 | AAYDLTGWFAYAV | SEQ ID NO: 128 |
| O30/MSLN | VL (light | QSVLTQPPSASGTPGQRVTISCSGSSSNIAHGPVNWYQQLPG | SEQ ID NO: 129 |
| | chain | TAPKLLIYATNHRPSGVPDRFSGSKSGTTASLTISGLQSEDE | |
| | variable | ADYYCAAYDLTGWFAYAVFGGGTKLTVLGQPKAAPSVTL | |
| | region) | | |
| O35/MSLN | LC (light | QPVLTQPVSLSASPGASVSLTCTLRSDIRVRDYRIFWYQQKP | SEQ ID NO: 130 |
| | chain) | GSPPQYLLRYKTDSDKQQGSGVPSRFSGSKDASANAGILLIS | |
| | | GLQSEDEADYYCMIWHRTTGTSLVFGGGTKLTVLGQPKAAPS | |
| | | VTLFPPSSEELQANKATLVCLISDFYPGAVTVAWKADSSPVK | |
| | | AGVETTTPSKQSNNKYAASSYLSLTPEQWKSHRSYSCQVTHE | |
| | | GSTVEKTVAPTECS | |
| O35/MSLN | CDR-L1 | SDIRVRDYR | SEQ ID NO: 131 |
| O35/MSLN | CDR-L2 | YKTDSDK | SEQ ID NO: 132 |
| O35/MSLN | CDR-L3 | MIWHRTTGTSLV | SEQ ID NO: 133 |
| O35/MSLN | VL (light | QPVLTQPVSLSASPGASVSLTCTLRSDIRVRDYRIFWYQQKP | SEQ ID NO: 134 |
| | chain | GSPPQYLLRYKTDSDKQQGSGVPSRFSGSKDASANAGILLIS | |
| | variable | GLQSEDEADYYCMIWHRTTGTSLVFGGGTKLTVL | |
| | region) | | |
| O38/MSLN | LC (light | QPVLTQPASLSASPGASASLTCTLRSGINVRDYRIFWYQQKP | SEQ ID NO: 135 |
| | chain) | GSPPQYLLRYKSASDKQQGSGVPSRFSGSKDASANAGILLIS | |
| | | GLQSEDEADYYCMIWHHDSEGHAFVFGGGTKLTVLGQPKAAP | |
| | | SVTLFPPSSEELQANKATLVCLISDFYPGAVTVAWKADSSPV | |
| | | KAGVETTTPSKQSNNKYAASSYLSLTPEQWKSHRSYSCQVTH | |
| | | EGSTVEKTVAPTECS | |
| O38/MSLN | CDR-L1 | SGINVRDYR | SEQ ID NO: 136 |
| O38/MSLN | CDR-L2 | YKSASDK | SEQ ID NO: 137 |
| O38/MSLN | CDR-L3 | MIWHHDSEGHAFV | SEQ ID NO: 138 |
| O38/MSLN | VL (light | QPVLTQPASLSASPGASASLTCTLRSGINVRDYRIFWYQQKP | SEQ ID NO: 139 |
| | chain | GSPPQYLLRYKSASDKQQGSGVPSRFSGSKDASANAGILLIS | |
| | variable | GLQSEDEADYYCMIWHHDSEGHAFVFGGGTKLTVL | |
| | region) | | |
| O41/MSLN | LC (light | SYVLTQPPSVSVAPGKTARITCGGNKIGHRAVHWYQQKPGQA | SEQ ID NO: 140 |
| | chain) | PVLVIYYTYERPSGIPERFSGSNSGNTATLTISRVEAGDEAD | |
| | | YYCQVWDWYSEGGVVFGGGTKLTVLGQPKAAPSVTLFPPSSE | |
| | | ELQANKATLVCLISDFYPGAVTVAWKADSSPVKAGVETTTPS | |
| | | KQSNNKYAASSYLSLTPEQWKSHRSYSCQVTHEGSTVEKTVA | |
| | | PTECS | |
| O41/MSLN | CDR-L1 | KIGHRA | SEQ ID NO: 141 |
| O41/MSLN | CDR-L2 | YTY | SEQ ID NO: 142 |
| O41/MSLN | CDR-L3 | QVWDWYSEGGVV | SEQ ID NO: 143 |
| O41/MSLN | VL (light | SYVLTQPPSVSVAPGKTARITCGGNKIGHRAVHWYQQKPGQA | SEQ ID NO: 144 |
| | chain | PVLVIYYTYERPSGIPERFSGSNSGNTATLTISRVEAGDEAD | |
| | variable | YYCQVWDWYSEGGVVFGGGTKLTVLGQPKAAPSVTL | |
| | region) | | |
| N2-19/HER2 | LC (light | QSVLTQPPSVSGAPGQRVTISCTGSSSNIENTHTVHWYQQLP | SEQ ID NO: 145 |
| | chain) | GTAPKLLIYDASIRPSGVPDRFSGSKSGTSASLAITGLQAED | |
| | | EADYYCQSYDWLRGAQVFGGGTKLTVLGQPKAAPSVTLFPPS | |
| | | SEELQANKATLVCLISDFYPGAVTVAWKADSSPVKAGVETTT | |
| | | PSKQSNNKYAASSYLSLTPEQWKSHRSYSCQVTHEGSTVEKT | |
| | | VAPTECS | |
| N2-19/HER2 | CDR-L1 | SSNIENTHT | SEQ ID NO: 146 |
| N2-19/HER2 | CDR-L2 | DAS | SEQ ID NO: 147 |
| N2-19/HER2 | CDR-L3 | QSYDWLRGAQV | SEQ ID NO: 148 |
| N2-19/HER2 | VL (light | QSVLTQPPSVSGAPGQRVTISCTGSSSNIENTHTVHWYQQLP | SEQ ID NO: 149 |
| | chain | GTAPKLLIYDASIRPSGVPDRFSGSKSGTSASLAITGLQAED | |
| | variable | EADYYCQSYDWLRGAQVFGGGTKLTVL | |
| | region) | | |
| N2-28/HER2 | LC (light | SYVLTQPPSVSVAPGKTARITCGGNTIGSTVVHWYQQKPGQA | SEQ ID NO: 150 |
| | chain) | PVLVIYFDDARPSGIPERFSGSNSGNTATLTISRVEAGDEAD | |
| | | YYCQVWDSFGLSLAPVFGGGTKLTVLGQPKAAPSVTLFPPSS | |
| | | EELQANKATLVCLISDFYPGAVTVAWKADSSPVKAGVETTTP | |
| | | SKQSNNKYAASSYLSLTPEQWKSHRSYSCQVTHEGSTVEKTV | |
| | | APTECS | |
| N2-28/HER2 | CDR-L1 | TIGSTV | SEQ ID NO: 151 |
| N2-28/HER2 | CDR-L2 | FDD | SEQ ID NO: 152 |
| N2-28/HER2 | CDR-L3 | QVWDSFGLSLAPV | SEQ ID NO: 153 |
| N2-28/HER2 | VL (light | SYVLTQPPSVSVAPGKTARITCGGNTIGSTVVHWYQQKPGQA | SEQ ID NO: 154 |
| | chain | PVLVIYFDDARPSGIPERFSGSNSGNTATLTISRVEAGDEAD | |
| | variable | YYCQVWDSFGLSLAPVFGGGTKLTVL | |
| | region) | | |

Binding arms can be combined in any combination to generate anti-IL2R×TAA bsAbs of the present invention. Exemplary bispecific antibodies of the disclosure are shown in Table 2. Each bispecific antibody within the matrix is a unique bsAb with a specific combination of antigen binding domains. The specific format (KiH or κλ-body) is also indicated. For example, the bispecific antibody “AL1N2-28” has a κλ-body format and comprises a “AL1” anti-IL2Rβ antigen binding domain and an “N2-28” anti-HER2 antigen binding domain. For example, the bispecific antibody “AM5O30” has a κλ-body format and comprises a “AM5” anti-IL2Rγ antigen binding domain and an “O30” anti-MSLN antigen binding domain.

Exemplary κλ-body format bispecific antibodies described herein include a common heavy chain (HC), one kappa chain or one lambda chain for anti-TAA and anti-first/second subunit of a cytokine receptor antigen binding domain, one kappa and one lambda light chains (LC) for anti-TAA and anti-first/second subunit of a cytokine receptor antigen binding domain, as shown in the amino acid sequences listed below. Each of the exemplary anti-TAA and anti-first/second subunit of a cytokine receptor bispecific antibodies described below includes a common variable heavy domain (VH), one kappa variable light domain or one lambda variable light domain for anti-TAA and anti-first/second subunit of a cytokine receptor antibodies, and one kappa and one lambda variable light domains (VL) for anti-TAA and anti-first/second subunit of a cytokine receptor antigen binding domains, as shown in the amino acid sequences listed below.

While antibody sequences below are provided herein as examples, it is to be understood that these sequences can be used to generate bispecific antibodies using any of a variety of art-recognized techniques. Examples of bispecific formats include but are not limited to bispecific IgG based on Fab arm exchange (Gramer et al., 2013 MAbs. 5(6)); the CrossMab format (Klein C et al., 2012 MAbs 4(6)); multiple formats based on forced heterodimerization approaches such as SEED technology (Davis J H et al., 2010 Protein Eng Des Sel. 23(4):195-202), electrostatic steering (Gunasekaran K et al., J Biol Chem. 2010 285(25):19637-46.) or knob-into-hole (Ridgway J B et al., Protein Eng. 1996 9(7):617-21.) or other sets of mutations preventing homodimer formation (Von Kreudenstein T S et al., 2013 MAbs. 5(5):646-54); fragment based bispecific formats such as tandem scFv (such asBiTEs) (Wolf E et al., 2005 Drug Discov. Today 10(18):1237-44); bispecific tetravalent antibodies (Portner L M et al., 2012 Cancer Immunol Immunother. 61(10):1869-75); dual affinity retargeting molecules (Moore P A et al., 2011 Blood. 117(17):4542-51), diabodies (Kontermann R E et al., Nat Biotechnol. 1997 15(7):629-31).

The bsAb antibodies of the composition can be generated using different formats and technologies, either relying on forced pairing of antibody chains to achieved bi-specificity (for example Knob-into-Hole/Cross-Mab technology) or using bsAb formats maintaining the native structure of human IgG (Kappa-Lambda Body technology).

TABLE 2
Nomenclature of Exemplary Bispecific Antibodies of the Disclosure

| | | HER2 antigen binding domain | | | | MSLN antigen binding domain | | | |
|---|---|---|---|---|---|---|---|---|---|
| | | Trastuzumab (*) | Pertuzumab (*) | N2-19 (#) | N2-28 (#) | O30 (#) | O35 (#) | O38 (#) | O41 (#) |
| IL2Rβ | P2C4 (*) | P2C4 × | P2C4 × | | | | | | |
| antigen | | Trastuzumab | Pertuzumab | | | | | | |
| binding | AL1 (#) | | | | AL1N2- | | | | |
| domain | | | | | 28 | | | | |
| | AL2 (#) | | | | AL2N2- | | | | |
| | | | | | 28 | | | | |
| | AL3 (#) | | | | AL3N2- | | | | |
| | | | | | 28 | | | | |
| | AL4 (#) | | | | AL4N2- | AL4O30 | AL4O35 | AL4O38 | AL4O41 |
| | | | | | 28 | | | | |
| | AL5 (#) | | | | AL5N2- | | | | |
| | | | | | 28 | | | | |
| IL2Rγ | P1A3 (*) | P1A3 × | P1A3 × | | | | | | |
| antigen | | Trastuzumab | Pertuzumab | | | | | | |
| binding | AM1 (#) | | | AM1N2- | | | | | |
| domain | | | | 19 | | | | | |
| | AM2 (#) | | | AM2N2- | | | | | |
| | | | | 19 | | | | | |
| | AM3 (#) | | | AM3N2- | | | | | |
| | | | | 19 | | | | | |
| | AM4 (#) | | | AM4N2- | | | | | |
| | | | | 19 | | | | | |
| | AM5 (#) | | | AM5N2- | | AM5O30 | AM5O35 | AM5O38 | AM5O41 |
| | | | | 19 | | | | | |
| | AM6 (#) | | | AM6N2- | | | | | |
| | | | | 19 | | | | | |
| | AM7 (#) | | | AM7N2- | | | | | |
| | | | | 19 | | | | | |
| | AM8 (#) | | | AM8N2- | | | | | |
| | | | | 19 | | | | | |
| | AM9 (#) | | | AM9N2- | | AM9O30 | | | |
| | | | | 19 | | | | | |
| | AM10 (#) | | | AM10N2- | | | | | |
| | | | | 19 | | | | | |
| | AM11 (#) | | | AM11N2- | | | | | |
| | | | | 19 | | | | | |

(*) Arms used to generate bsAb in a CrossMab-KiH format
(#) Arms used to generate bsAb in a κλ-body format.
Each bsAb was generated using an inactive Fc (LALA + P329A mutation) depicted as “/N” throughout in the application and figure legends.
A anti-IL2Rβ × anti-IL2Rγ bispecific antibody was generated by crossing the P1A3 arm with the P2C4 arm.

### Exemplary Anti-IL2Rγ and Anti-HER2 Bispecific Antibodies

In some embodiments, the bispecific antibody P1A3× Trastuzumab comprises a P1A3 antigen binding domain that comprises a CDRH1 comprising the amino acid sequence of SEQ ID NO: 19, a CDRH2 comprising the amino acid sequence of SEQ ID NO: 20, a CDRH3 comprising the amino acid sequence of SEQ ID NO: 21, a CDRL1 comprising the amino acid sequence of SEQ ID NO: 15, a CDRL2 comprising the amino acid sequence of SEQ ID NO: 16, and a CDRL3 comprising the amino acid sequence of SEQ ID NO: 17; and a Trastuzumab antigen binding domain that comprises a a CDRH1 comprising the amino acid sequence of SEQ ID NO: 27, a CDRH2 comprising the amino acid sequence of SEQ ID NO: 28, a CDRH3 comprising the amino acid sequence of SEQ ID NO: 29, a CDRL1 comprising the amino acid sequence of SEQ ID NO: 23, a CDRL2 comprising the amino acid sequence of SEQ ID NO: 24, and a CDRL3 comprising the amino acid sequence of SEQ ID NO: 25.

In some embodiments, the bispecific antibody P1A3× Trastuzumab comprises a P1A3 antigen binding domain that comprises a heavy chain comprising the amino acid sequence of SEQ ID NO: 18, and a light chain comprising the amino acid sequence of SEQ ID NO: 14; and a Trastuzumab antigen binding domain that a heavy chain comprising the amino acid sequence of SEQ ID NO: 26, and a light chain comprising the amino acid sequence of SEQ ID NO: 22.

In some embodiments, the bispecific antibody P1A3× Pertuzumab comprises a P1A3 antigen binding domain that comprises a CDRH1 comprising the amino acid sequence of SEQ ID NO: 19, a CDRH2 comprising the amino acid sequence of SEQ ID NO: 20, a CDRH3 comprising the amino acid sequence of SEQ ID NO: 21, a CDRL1 comprising the amino acid sequence of SEQ ID NO: 15, a CDRL2 comprising the amino acid sequence of SEQ ID NO: 16, and a CDRL3 comprising the amino acid sequence of SEQ ID NO: 17; and a Pertuzumab antigen binding domain that comprises a a CDRH1 comprising the amino acid sequence of SEQ ID NO: 35, a CDRH2 comprising the amino acid sequence of SEQ ID NO: 36, a CDRH3 comprising the amino acid sequence of SEQ ID NO: 37, a CDRL1 comprising the amino acid sequence of SEQ ID NO: 31, a CDRL2 comprising the amino acid sequence of SEQ ID NO: 32, and a CDRL3 comprising the amino acid sequence of SEQ ID NO: 33.

In some embodiments, the bispecific antibody P1A3× Pertuzumab comprises a P1A3 antigen binding domain that comprises a heavy chain comprising the amino acid sequence of SEQ ID NO: 18, and a light chain comprising the amino acid sequence of SEQ ID NO: 14; and a Pertuzumab antigen binding domain that a heavy chain comprising the amino acid sequence of SEQ ID NO: 34, and a light chain comprising the amino acid sequence of SEQ ID NO: 30.

In some embodiments, the bispecific antibody AM1N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 64, a CDRL2 comprising the amino acid sequence of SEQ ID NO: 65, and a CDRL3 comprising the amino acid sequence of SEQ ID NO: 66, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM1N2-19 comprises a common heavy chain variable region comprising the amino acid sequence of SEQ ID NO: 4, a kappa light chain variable region comprising the amino acid sequence of SEQ ID NO: 67, and a lambda light chain variable region comprising the amino acid sequence of SEQ ID NO: 149.

In some embodiments, the bispecific antibody AM1N2-19 comprises a common heavy chain comprising the amino acid sequence of SEQ ID NO: 5, a kappa light chain comprising the amino acid sequence of SEQ ID NO: 63, and a lambda light chain comprising the amino acid sequence of SEQ ID NO: 145.

In some embodiments, the bispecific antibody AM2N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 69, a CDRL2 comprising the amino acid sequence of SEQ ID NO: 70, and a CDRL3 comprising the amino acid sequence of SEQ ID NO: 71, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM2N2-19 comprises a common heavy chain variable region comprising the amino acid sequence of SEQ ID NO: 4, a kappa light chain variable region comprising the amino acid sequence of SEQ ID NO: 72, and a lambda light chain variable region comprising the amino acid sequence of SEQ ID NO: 149.

In some embodiments, the bispecific antibody AM2N2-19 comprises a common heavy chain comprising the amino acid sequence of SEQ ID NO: 5, a kappa light chain comprising the amino acid sequence of SEQ ID NO: 68, and a lambda light chain comprising the amino acid sequence of SEQ ID NO: 145.

In some embodiments, the bispecific antibody AM3N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 74, a CDRL2 comprising the amino acid sequence of SEQ ID NO: 75, and a CDRL3 comprising the amino acid sequence of SEQ ID NO: 76, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM3N2-19 comprises a common heavy chain variable region comprising the amino acid sequence of SEQ ID NO: 4, a kappa light chain variable region comprising the amino acid sequence of SEQ ID NO: 77, and a lambda light chain variable region comprising the amino acid sequence of SEQ ID NO: 149.

In some embodiments, the bispecific antibody AM3N2-19 comprises a common heavy chain comprising the amino acid sequence of SEQ ID NO: 5, a kappa light chain comprising the amino acid sequence of SEQ ID NO: 73, and a lambda light chain comprising the amino acid sequence of SEQ ID NO: 145.

In some embodiments, the bispecific antibody AM4N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 79, a CDRL2 comprising the amino acid sequence of SEQ ID NO: 80, and a CDRL3 comprising the amino acid sequence of SEQ ID NO: 81, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM4N2-19 comprises a common heavy chain variable region comprising the amino acid sequence of SEQ ID NO: 4, a kappa light chain variable region comprising the amino acid sequence of SEQ ID NO: 82, and a lambda light chain variable region comprising the amino acid sequence of SEQ ID NO: 149.

In some embodiments, the bispecific antibody AM4N2-19 comprises a common heavy chain comprising the amino acid sequence of SEQ ID NO: 5, a kappa light chain comprising the amino acid sequence of SEQ ID NO: 78, and a lambda light chain comprising the amino acid sequence of SEQ ID NO: 145.

In some embodiments, the bispecific antibody AM5N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 84, a CDRL2 comprising the amino acid sequence of SEQ ID NO: 85, and a CDRL3 comprising the amino acid sequence of SEQ ID NO: 86, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM5N2-19 comprises a common heavy chain variable region comprising the amino acid sequence of SEQ ID NO: 4, a kappa light chain variable region comprising the amino acid sequence of SEQ ID NO: 87, and a lambda light chain variable region comprising the amino acid sequence of SEQ ID NO: 149.

In some embodiments, the bispecific antibody AM5N2-19 comprises a common heavy chain comprising the amino acid sequence of SEQ ID NO: 5, a kappa light chain comprising the amino acid sequence of SEQ ID NO: 83, and a lambda light chain comprising the amino acid sequence of SEQ ID NO: 145.

In some embodiments, the bispecific antibody AM6N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 89, a CDRL2 comprising the amino acid sequence of SEQ ID NO: 90, and a CDRL3 comprising the amino acid sequence of SEQ ID NO: 91, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM6N2-19 comprises a common heavy chain variable region comprising the amino acid sequence of SEQ ID NO: 4, a kappa light chain variable region comprising the amino acid sequence of SEQ ID NO: 92, and a lambda light chain variable region comprising the amino acid sequence of SEQ ID NO: 149.

In some embodiments, the bispecific antibody AM6N2-19 comprises a common heavy chain comprising the amino acid sequence of SEQ ID NO: 5, a kappa light chain comprising the amino acid sequence of SEQ ID NO: 88, and a lambda light chain comprising the amino acid sequence of SEQ ID NO: 145.

In some embodiments, the bispecific antibody AM7N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 94, a CDRL2 comprising the amino acid sequence of SEQ ID NO: 95, and a CDRL3 comprising the amino acid sequence of SEQ ID NO: 96, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM7N2-19 comprises a common heavy chain variable region comprising the amino acid sequence of SEQ ID NO: 4, a kappa light chain variable region comprising the amino acid sequence of SEQ ID NO: 97, and a lambda light chain variable region comprising the amino acid sequence of SEQ ID NO: 149.

In some embodiments, the bispecific antibody AM7N2-19 comprises a common heavy chain comprising the amino acid sequence of SEQ ID NO: 5, a kappa light chain comprising the amino acid sequence of SEQ ID NO: 93, and a lambda light chain comprising the amino acid sequence of SEQ ID NO: 145.

In some embodiments, the bispecific antibody AM8N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 99, a CDRL2 comprising the amino acid sequence of SEQ ID NO: 100, and a CDRL3 comprising the amino acid sequence of SEQ ID NO: 101, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM8N2-19 comprises a common heavy chain variable region comprising the amino acid sequence of SEQ ID NO: 4, a kappa light chain variable region comprising the amino acid sequence of SEQ ID NO: 102, and a lambda light chain variable region comprising the amino acid sequence of SEQ ID NO: 149.

In some embodiments, the bispecific antibody AM8N2-19 comprises a common heavy chain comprising the amino acid sequence of SEQ ID NO: 5, a kappa light chain comprising the amino acid sequence of SEQ ID NO: 98, and a lambda light chain comprising the amino acid sequence of SEQ ID NO: 145.

In some embodiments, the bispecific antibody AM9N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 104, a CDRL2 comprising the amino acid sequence of SEQ ID NO: 105, and a CDRL3 comprising the amino acid sequence of SEQ ID NO: 106, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM9N2-19 comprises a common heavy chain variable region comprising the amino acid sequence of SEQ ID NO: 4, a kappa light chain variable region comprising the amino acid sequence of SEQ ID NO: 107, and a lambda light chain variable region comprising the amino acid sequence of SEQ ID NO: 149.

In some embodiments, the bispecific antibody AM9N2-19 comprises a common heavy chain comprising the amino acid sequence of SEQ ID NO: 5, a kappa light chain comprising the amino acid sequence of SEQ ID NO: 103, and a lambda light chain comprising the amino acid sequence of SEQ ID NO: 145.

In some embodiments, the bispecific antibody AM10N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 109, a CDRL2 comprising the amino acid sequence of SEQ ID NO: 110, and a CDRL3 comprising the amino acid sequence of SEQ ID NO: 111, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM10N2-19 comprises a common heavy chain variable region comprising the amino acid sequence of SEQ ID NO: 4, a kappa light chain variable region comprising the amino acid sequence of SEQ ID NO: 112, and a lambda light chain variable region comprising the amino acid sequence of SEQ ID NO: 149.

In some embodiments, the bispecific antibody AM10N2-19 comprises a common heavy chain comprising the amino acid sequence of SEQ ID NO: 5, a kappa light chain comprising the amino acid sequence of SEQ ID NO: 108, and a lambda light chain comprising the amino acid sequence of SEQ ID NO: 145.

In some embodiments, the bispecific antibody AM11N2-19 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 114, a CDRL2 comprising the amino acid sequence of SEQ ID NO: 115, and a CDRL3 comprising the amino acid sequence of SEQ ID NO: 116, and a lambda light chain comprising a CDRL1 comprising the amino acid sequence of SEQ ID NO: 146, a CDRL2 comprising the amino acid sequence of SEQ ID NO: 147, and a CDRL3 comprising the amino acid sequence of SEQ ID NO: 148.

In some embodiments, the bispecific antibody AM11N2-19 comprises a common heavy chain variable region comprising the amino acid sequence of SEQ ID NO: 4, a kappa light chain variable region comprising the amino acid sequence of SEQ ID NO: 117, and a lambda light chain variable region comprising the amino acid sequence of SEQ ID NO: 149.

In some embodiments, the bispecific antibody AM11N2-19 comprises a common heavy chain comprising the amino acid sequence of SEQ ID NO: 5, a kappa light chain comprising the amino acid sequence of SEQ ID NO: 113, and a lambda light chain comprising the amino acid sequence of SEQ ID NO: 145.

### Exemplary Anti-IL2Rβ and Anti-HER2 Bispecific Antibodies

In some embodiments, the bispecific antibody P2C4× Trastuzumab comprises a P2C4 antigen binding domain that comprises a CDRH1 comprising the amino acid sequence of SEQ ID NO: 11, a CDRH2 comprising the amino acid sequence of SEQ ID NO: 12, a CDRH3 comprising the amino acid sequence of SEQ ID NO: 13, a CDRL1 comprising the amino acid sequence of SEQ ID NO: 7, a CDRL2 comprising the amino acid sequence of SEQ ID NO: 8, and a CDRL3 comprising the amino acid sequence of SEQ ID NO: 9; and a Trastuzumab antigen binding domain that comprises a a CDRH1 comprising the amino acid sequence of SEQ ID NO: 27, a CDRH2 comprising the amino acid sequence of SEQ ID NO: 28, a CDRH3 comprising the amino acid sequence of SEQ ID NO: 29, a CDRL1 comprising the amino acid sequence of SEQ ID NO: 23, a CDRL2 comprising the amino acid sequence of SEQ ID NO: 24, and a CDRL3 comprising the amino acid sequence of SEQ ID NO: 25.

In some embodiments, the bispecific antibody P2C4× Trastuzumab comprises a P2C4 antigen binding domain that comprises a heavy chain comprising the amino acid sequence of SEQ ID NO: 10, and a light chain comprising the amino acid sequence of SEQ ID NO: 6; and a Trastuzumab antigen binding domain that a heavy chain comprising the amino acid sequence of SEQ ID NO: 26, and a light chain comprising the amino acid sequence of SEQ ID NO: 22.

In some embodiments, the bispecific antibody P2C4× Pertuzumab comprises a P2C4 antigen binding domain that comprises a CDRH1 comprising the amino acid sequence of SEQ ID NO: 11, a CDRH2 comprising the amino acid sequence of SEQ ID NO: 12, a CDRH3 comprising the amino acid sequence of SEQ ID NO: 13, a CDRL1 comprising the amino acid sequence of SEQ ID NO: 7, a CDRL2 comprising the amino acid sequence of SEQ ID NO: 8, and a CDRL3 comprising the amino acid sequence of SEQ ID NO: 9; and a Pertuzumab antigen binding domain that comprises a a CDRH1 comprising the amino acid sequence of SEQ ID NO: 35, a CDRH2 comprising the amino acid sequence of SEQ ID NO: 36, a CDRH3 comprising the amino acid sequence of SEQ ID NO: 37, a CDRL1 comprising the amino acid sequence of SEQ ID NO: 31, a CDRL2 comprising the amino acid sequence of SEQ ID NO: 32, and a CDRL3 comprising the amino acid sequence of SEQ ID NO: 33.

In some embodiments, the bispecific antibody P2C4× Pertuzumab comprises a P2C4 antigen binding domain that comprises a heavy chain comprising the amino acid sequence of SEQ ID NO: 10, and a light chain comprising the amino acid sequence of SEQ ID NO: 6; and a Pertuzumab antigen binding domain that a heavy chain comprising the amino acid sequence of SEQ ID NO: 34, and a light chain comprising the amino acid sequence of SEQ ID NO: 30.

In some embodiments, the bispecific antibody AL1N2-28 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 39, a CDRL2 comprising the amino acid sequence of SEQ ID NO: 40, and a CDRL3 comprising the amino acid sequence of SEQ ID NO: 41, and a lambda light chain comprising a CDRL1 comprising the amino acid sequence of SEQ ID NO: 151, a CDRL2 comprising the amino acid sequence of SEQ ID NO: 152, and a CDRL3 comprising the amino acid sequence of SEQ ID NO: 153.

In some embodiments, the bispecific antibody AL1N2-28 comprises a common heavy chain variable region comprising the amino acid sequence of SEQ ID NO: 4, a kappa light chain variable region comprising the amino acid sequence of SEQ ID NO: 42, and a lambda light chain variable region comprising the amino acid sequence of SEQ ID NO: 154.

In some embodiments, the bispecific antibody AL1N2-28 comprises a common heavy chain comprising the amino acid sequence of SEQ ID NO: 5, a kappa light chain comprising the amino acid sequence of SEQ ID NO: 38, and a lambda light chain comprising the amino acid sequence of SEQ ID NO: 150.

In some embodiments, the bispecific antibody AL2N2-28 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 44, a CDRL2 comprising the amino acid sequence of SEQ ID NO: 45, and a CDRL3 comprising the amino acid sequence of SEQ ID NO: 46, and a lambda light chain comprising a CDRL1 comprising the amino acid sequence of SEQ ID NO: 151, a CDRL2 comprising the amino acid sequence of SEQ ID NO: 152, and a CDRL3 comprising the amino acid sequence of SEQ ID NO: 153.

In some embodiments, the bispecific antibody AL2N2-28 comprises a common heavy chain variable region comprising the amino acid sequence of SEQ ID NO: 4, a kappa light chain variable region comprising the amino acid sequence of SEQ ID NO: 47, and a lambda light chain variable region comprising the amino acid sequence of SEQ ID NO: 154.

In some embodiments, the bispecific antibody AL2N2-28 comprises a common heavy chain comprising the amino acid sequence of SEQ ID NO: 5, a kappa light chain comprising the amino acid sequence of SEQ ID NO: 43, and a lambda light chain comprising the amino acid sequence of SEQ ID NO: 150.

In some embodiments, the bispecific antibody AL3N2-28 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 49, a CDRL2 comprising the amino acid sequence of SEQ ID NO: 50, and a CDRL3 comprising the amino acid sequence of SEQ ID NO: 51, and a lambda light chain comprising a CDRL1 comprising the amino acid sequence of SEQ ID NO: 151, a CDRL2 comprising the amino acid sequence of SEQ ID NO: 152, and a CDRL3 comprising the amino acid sequence of SEQ ID NO: 153.

In some embodiments, the bispecific antibody AL3N2-28 comprises a common heavy chain variable region comprising the amino acid sequence of SEQ ID NO: 4, a kappa light chain variable region comprising the amino acid sequence of SEQ ID NO: 52, and a lambda light chain variable region comprising the amino acid sequence of SEQ ID NO: 154.

In some embodiments, the bispecific antibody AL3N2-28 comprises a common heavy chain comprising the amino acid sequence of SEQ ID NO: 5, a kappa light chain comprising the amino acid sequence of SEQ ID NO: 48, and a lambda light chain comprising the amino acid sequence of SEQ ID NO: 150.

In some embodiments, the bispecific antibody AL4N2-28 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 54, a CDRL2 comprising the amino acid sequence of SEQ ID NO: 55, and a CDRL3 comprising the amino acid sequence of SEQ ID NO: 56, and a lambda light chain comprising a CDRL1 comprising the amino acid sequence of SEQ ID NO: 151, a CDRL2 comprising the amino acid sequence of SEQ ID NO: 152, and a CDRL3 comprising the amino acid sequence of SEQ ID NO: 153.

In some embodiments, the bispecific antibody AL4N2-28 comprises a common heavy chain variable region comprising the amino acid sequence of SEQ ID NO: 4, a kappa light chain variable region comprising the amino acid sequence of SEQ ID NO: 57, and a lambda light chain variable region comprising the amino acid sequence of SEQ ID NO: 154.

In some embodiments, the bispecific antibody AL4N2-28 comprises a common heavy chain comprising the amino acid sequence of SEQ ID NO: 5, a kappa light chain comprising the amino acid sequence of SEQ ID NO: 53, and a lambda light chain comprising the amino acid sequence of SEQ ID NO: 150.

In some embodiments, the bispecific antibody AL5N2-28 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 59, a CDRL2 comprising the amino acid sequence of SEQ ID NO: 60, and a CDRL3 comprising the amino acid sequence of SEQ ID NO: 61, and a lambda light chain comprising a CDRL1 comprising the amino acid sequence of SEQ ID NO: 151, a CDRL2 comprising the amino acid sequence of SEQ ID NO: 152, and a CDRL3 comprising the amino acid sequence of SEQ ID NO: 153.

In some embodiments, the bispecific antibody AL5N2-28 comprises a common heavy chain variable region comprising the amino acid sequence of SEQ ID NO: 4, a kappa light chain variable region comprising the amino acid sequence of SEQ ID NO: 62, and a lambda light chain variable region comprising the amino acid sequence of SEQ ID NO: 154.

In some embodiments, the bispecific antibody AL5N2-28 comprises a common heavy chain comprising the amino acid sequence of SEQ ID NO: 5, a kappa light chain comprising the amino acid sequence of SEQ ID NO: 58, and a lambda light chain comprising the amino acid sequence of SEQ ID NO: 150.

### Exemplary Anti-IL2Rγ and Anti-HER2 Bispecific Antibodies

In some embodiments, the bispecific antibody AM5O30 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 84, a CDRL2 comprising the amino acid sequence of SEQ ID NO: 85, and a CDRL3 comprising the amino acid sequence of SEQ ID NO: 86, and a lambda light chain comprising a CDRL1 comprising the amino acid sequence of SEQ ID NO: 126, a CDRL2 comprising the amino acid sequence of SEQ ID NO: 127, and a CDRL3 comprising the amino acid sequence of SEQ ID NO: 128.

In some embodiments, the bispecific antibody AM5O30 comprises a common heavy chain variable region comprising the amino acid sequence of SEQ ID NO: 4, a kappa light chain variable region comprising the amino acid sequence of SEQ ID NO: 87, and a lambda light chain variable region comprising the amino acid sequence of SEQ ID NO: 129.

In some embodiments, the bispecific antibody AM5O30 comprises a common heavy chain comprising the amino acid sequence of SEQ ID NO: 5, a kappa light chain comprising the amino acid sequence of SEQ ID NO: 83, and a lambda light chain comprising the amino acid sequence of SEQ ID NO: 125.

In some embodiments, the bispecific antibody AM5O35 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 84, a CDRL2 comprising the amino acid sequence of SEQ ID NO: 85, and a CDRL3 comprising the amino acid sequence of SEQ ID NO: 86, and a lambda light chain comprising a CDRL1 comprising the amino acid sequence of SEQ ID NO: 131, a CDRL2 comprising the amino acid sequence of SEQ ID NO: 132, and a CDRL3 comprising the amino acid sequence of SEQ ID NO: 133.

In some embodiments, the bispecific antibody AM5O35 comprises a common heavy chain variable region comprising the amino acid sequence of SEQ ID NO: 4, a kappa light chain variable region comprising the amino acid sequence of SEQ ID NO: 87, and a lambda light chain variable region comprising the amino acid sequence of SEQ ID NO: 134.

In some embodiments, the bispecific antibody AM5O35 comprises a common heavy chain comprising the amino acid sequence of SEQ ID NO: 5, a kappa light chain comprising the amino acid sequence of SEQ ID NO: 83, and a lambda light chain comprising the amino acid sequence of SEQ ID NO: 130.

In some embodiments, the bispecific antibody AM5O38 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 84, a CDRL2 comprising the amino acid sequence of SEQ ID NO: 85, and a CDRL3 comprising the amino acid sequence of SEQ ID NO: 86, and a lambda light chain comprising a CDRL1 comprising the amino acid sequence of SEQ ID NO: 136, a CDRL2 comprising the amino acid sequence of SEQ ID NO: 137, and a CDRL3 comprising the amino acid sequence of SEQ ID NO: 138.

In some embodiments, the bispecific antibody AM5O38 comprises a common heavy chain variable region comprising the amino acid sequence of SEQ ID NO: 4, a kappa light chain variable region comprising the amino acid sequence of SEQ ID NO: 87, and a lambda light chain variable region comprising the amino acid sequence of SEQ ID NO: 139.

In some embodiments, the bispecific antibody AM5O38 comprises a common heavy chain comprising the amino acid sequence of SEQ ID NO: 5, a kappa light chain comprising the amino acid sequence of SEQ ID NO: 83, and a lambda light chain comprising the amino acid sequence of SEQ ID NO: 135.

In some embodiments, the bispecific antibody AM5O41 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 84, a CDRL2 comprising the amino acid sequence of SEQ ID NO: 85, and a CDRL3 comprising the amino acid sequence of SEQ ID NO: 86, and a lambda light chain comprising a CDRL1 comprising the amino acid sequence of SEQ ID NO: 141, a CDRL2 comprising the amino acid sequence of SEQ ID NO: 142, and a CDRL3 comprising the amino acid sequence of SEQ ID NO: 143.

In some embodiments, the bispecific antibody AM5O41 comprises a common heavy chain variable region comprising the amino acid sequence of SEQ ID NO: 4, a kappa light chain variable region comprising the amino acid sequence of SEQ ID NO: 87, and a lambda light chain variable region comprising the amino acid sequence of SEQ ID NO: 144.

In some embodiments, the bispecific antibody AM5O41 comprises a common heavy chain comprising the amino acid sequence of SEQ ID NO: 5, a kappa light chain comprising the amino acid sequence of SEQ ID NO: 83, and a lambda light chain comprising the amino acid sequence of SEQ ID NO: 140.

In some embodiments, the bispecific antibody AM9O30 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 104, a CDRL2 comprising the amino acid sequence of SEQ ID NO: 105, and a CDRL3 comprising the amino acid sequence of SEQ ID NO: 106, and a lambda light chain comprising a CDRL1 comprising the amino acid sequence of SEQ ID NO: 126, a CDRL2 comprising the amino acid sequence of SEQ ID NO: 127, and a CDRL3 comprising the amino acid sequence of SEQ ID NO: 128.

In some embodiments, the bispecific antibody AM9O30 comprises a common heavy chain variable region comprising the amino acid sequence of SEQ ID NO: 4, a kappa light chain variable region comprising the amino acid sequence of SEQ ID NO: 107, and a lambda light chain variable region comprising the amino acid sequence of SEQ ID NO: 129.

In some embodiments, the bispecific antibody AM9O30 comprises a common heavy chain comprising the amino acid sequence of SEQ ID NO: 5, a kappa light chain comprising the amino acid sequence of SEQ ID NO: 103, and a lambda light chain comprising the amino acid sequence of SEQ ID NO: 125.

### Exemplary Anti-IL2Rβ and Anti-HER2 Bispecific Antibodies

In some embodiments, the bispecific antibody AL4O30 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 54, a CDRL2 comprising the amino acid sequence of SEQ ID NO: 55, and a CDRL3 comprising the amino acid sequence of SEQ ID NO: 56, and a lambda light chain comprising a CDRL1 comprising the amino acid sequence of SEQ ID NO: 126, a CDRL2 comprising the amino acid sequence of SEQ ID NO: 127, and a CDRL3 comprising the amino acid sequence of SEQ ID NO: 128.

In some embodiments, the bispecific antibody AL4O30 comprises a common heavy chain variable region comprising the amino acid sequence of SEQ ID NO: 4, a kappa light chain variable region comprising the amino acid sequence of SEQ ID NO: 57, and a lambda light chain variable region comprising the amino acid sequence of SEQ ID NO: 129.

In some embodiments, the bispecific antibody AL4O30 comprises a common heavy chain comprising the amino acid sequence of SEQ ID NO: 5, a kappa light chain comprising the amino acid sequence of SEQ ID NO: 53, and a lambda light chain comprising the amino acid sequence of SEQ ID NO: 125.

In some embodiments, the bispecific antibody AL4O35 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 54, a CDRL2 comprising the amino acid sequence of SEQ ID NO: 55, and a CDRL3 comprising the amino acid sequence of SEQ ID NO: 56, and a lambda light chain comprising a CDRL1 comprising the amino acid sequence of SEQ ID NO: 131, a CDRL2 comprising the amino acid sequence of SEQ ID NO: 132, and a CDRL3 comprising the amino acid sequence of SEQ ID NO: 133.

In some embodiments, the bispecific antibody AL4O35 comprises a common heavy chain variable region comprising the amino acid sequence of SEQ ID NO: 4, a kappa light chain variable region comprising the amino acid sequence of SEQ ID NO: 57, and a lambda light chain variable region comprising the amino acid sequence of SEQ ID NO: 134.

In some embodiments, the bispecific antibody AL4O35 comprises a common heavy chain comprising the amino acid sequence of SEQ ID NO: 5, a kappa light chain comprising the amino acid sequence of SEQ ID NO: 53, and a lambda light chain comprising the amino acid sequence of SEQ ID NO: 130.

In some embodiments, the bispecific antibody AL4O38 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 54, a CDRL2 comprising the amino acid sequence of SEQ ID NO: 55, and a CDRL3 comprising the amino acid sequence of SEQ ID NO: 56, and a lambda light chain comprising a CDRL1 comprising the amino acid sequence of SEQ ID NO: 136, a CDRL2 comprising the amino acid sequence of SEQ ID NO: 137, and a CDRL3 comprising the amino acid sequence of SEQ ID NO: 138.

In some embodiments, the bispecific antibody AL4O38 comprises a common heavy chain variable region comprising the amino acid sequence of SEQ ID NO: 4, a kappa light chain variable region comprising the amino acid sequence of SEQ ID NO: 57, and a lambda light chain variable region comprising the amino acid sequence of SEQ ID NO: 139.

In some embodiments, the bispecific antibody AL4O38 comprises a common heavy chain comprising the amino acid sequence of SEQ ID NO: 5, a kappa light chain comprising the amino acid sequence of SEQ ID NO: 53, and a lambda light chain comprising the amino acid sequence of SEQ ID NO: 135.

In some embodiments, the bispecific antibody AL4O41 comprises a common heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 54, a CDRL2 comprising the amino acid sequence of SEQ ID NO: 55, and a CDRL3 comprising the amino acid sequence of SEQ ID NO: 56, and a lambda light chain comprising a CDRL1 comprising the amino acid sequence of SEQ ID NO: 141, a CDRL2 comprising the amino acid sequence of SEQ ID NO: 142, and a CDRL3 comprising the amino acid sequence of SEQ ID NO: 143.

In some embodiments, the bispecific antibody AL4O41 comprises a common heavy chain variable region comprising the amino acid sequence of SEQ ID NO: 4, a kappa light chain variable region comprising the amino acid sequence of SEQ ID NO: 57, and a lambda light chain variable region comprising the amino acid sequence of SEQ ID NO: 144.

In some embodiments, the bispecific antibody AL4O41 comprises a common heavy chain comprising the amino acid sequence of SEQ ID NO: 5, a kappa light chain comprising the amino acid sequence of SEQ ID NO: 53, and a lambda light chain comprising the amino acid sequence of SEQ ID NO: 140.

### Methods of Use

This disclosure provides the use of a composition comprising: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor. In some aspects, the composition is used for inhibiting tumor growth. In some aspects, the composition is used for the treatment of cancer. In some aspects, the composition is used for enhancing T-cell mediated cell killing. In some aspects, the composition is used for T-cell activation.

The present disclosure provides methods for treating, preventing or alleviating at least one symptom of a cell proliferative disorder in a subject in need thereof. In some aspects the method is alleviating at least one symptom of a cell proliferative disorder in a subject in need thereof. In one aspect the cell proliferative disorder is cancer.

In some aspects, the methods for treating, preventing or alleviating at least one symptom of a cell proliferative disorder in a subject in need thereof comprising administering a composition comprising: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor.

In some aspects, the first bispecific antibody and the second bispecific antibody are administered separately to a subject. In some aspects, the first bispecific antibody and the second antibody are formulated separately and are combined prior to the administration to a subject. In some embodiments, the first bispecific antibody and the second bispecific antibody is co-formulated and administered to a subject.

As used herein, a “subject” can be any mammal, e.g., a human, a primate, mouse, rat, dog, cat, cow, horse, pig, sheep, goat, camel. In a preferred aspect, the subject is a human.

In some aspects a “subject in need thereof” is a subject having a cell proliferative disorder, or a subject having an increased risk of developing a cell proliferative disorder relative to the population at large. In some aspects a subject in need thereof has a precancerous condition. In one aspect, a subject in need thereof has cancer.

As used herein, “treating” describes the management and care of a patient for the purpose of combating a disease, condition, or disorder and includes decreasing or alleviating the symptoms or complications, or eliminating the disease, condition or disorder. As used herein, “preventing” describes stopping the onset of the symptoms or complications of the disease, condition or disorder. As used herein, “alleviating” describes reducing the symptoms or complications of disease, condition or disorder.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the disclosure encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi's sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. Exemplary cancers include, but are not limited to, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma.

A therapeutic regimen is carried out by identifying a subject, e.g., a human patient suffering from (or at risk of developing) a cancer, using standard methods. Efficaciousness treatment is determined in association with any known method for diagnosing or treating the particular immune-related disorder. Alleviation of one or more symptoms of the immune-related disorder indicates that the antibody confers a clinical benefit.

In some aspects treating cancer results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement.

In some aspects, treating cancer results in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In some aspects, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement.

In some aspects, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement.

In some aspects, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In some aspects, an increase in average survival time of a population may be measured, for example, by calculating for a population the average length of survival following initiation of treatment with an active compound. In some aspects, an increase in average survival time of a population may also be measured, for example, by calculating for a population the average length of survival following completion of a first round of treatment with an active compound.

In some aspects, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In some aspects, treating cancer results in increase in average survival time of a population of treated subjects in comparison to a population receiving a therapy that is not a recombinant polypeptide of the present disclosure. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In some aspects, treating cancer results in a decrease in the mortality rate of a population of treated subjects in comparison to a population receiving carrier alone. In some aspects, treating cancer results in a decrease in the mortality rate of a population of treated subjects in comparison to an untreated population. In a further aspect, treating cancer results in a decrease in the mortality rate of a population of treated subjects in comparison to a population receiving monotherapy with a drug that is not a recombinant polypeptide of the present disclosure. Preferably, the mortality rate is decreased by more than 2%; more preferably, by more than 5%; more preferably, by more than 10%; and most preferably, by more than 25%. In some aspects, a decrease in the mortality rate of a population of treated subjects may be measured by any reproducible means. In some aspects, a decrease in the mortality rate of a population may be measured, for example, by calculating for a population the average number of disease-related deaths per unit time following initiation of treatment with an active compound. In some aspects, a decrease in the mortality rate of a population may also be measured, for example, by calculating for a population the average number of disease-related deaths per unit time following completion of a first round of treatment with an active compound.

In some aspects, treating cancer results in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In some aspects, tumor growth rate is measured according to a change in tumor diameter per unit time.

In some aspects, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In some aspects, tumor regrowth is measured, for example, by measuring an increase in the diameter of a tumor after a prior tumor shrinkage that followed treatment. In some aspects, a decrease in tumor regrowth is indicated by failure of tumors to reoccur after treatment has stopped.

In some aspects, treating, preventing, or alleviating a cancer results in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. In some aspects, the rate of cellular proliferation is measured, for example, by measuring the number of dividing cells in a tissue sample per unit time.

In some aspects, treating, preventing, or alleviating a cancer results in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. In some aspects, the proportion of proliferating cells is measured, for example, by quantifying the number of dividing cells relative to the number of nondividing cells in a tissue sample. In some aspects, the proportion of proliferating cells is equivalent to the mitotic index.

In some aspects, treating, preventing, or alleviating a cancer results in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. In some aspects, size of an area or zone of cellular proliferation may be measured as a diameter or width of an area or zone of cellular proliferation.

In some aspects, treating, preventing, or alleviating a cancer results in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. In some aspects an abnormal cellular morphology is measured by microscopy, e.g., using an inverted tissue culture microscope. In some aspects an abnormal cellular morphology takes the form of nuclear pleiomorphism.

In some aspects treating cancer or a cell proliferative disorder results in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. In some aspects, number of cells in a population is measured by fluorescence activated cell sorting (FACS). In some aspects, number of cells in a population is measured by immunofluorescence microscopy. In some aspects, number of cells in a population is measured by light microscopy. In some aspects, methods of measuring cell death are as shown in Li et al., (2003) Proc Natl Acad Sci USA. 100(5): 2674-8.

The present disclosure provides methods of enhancing immune-cell mediated cell killing comprising providing to a population of cells, a composition comprising: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor. In some aspects, the disclosure provides methods of enhancing T-cell mediated cell killing. In some aspects, T-cell mediated cell killing results in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means.

The present disclosure provides methods for enhancing immune cell activation comprising providing to a population of immune cells, a composition comprising: a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor. In some aspects, the disclosure provide methods for enhancing T-cell activation. In some aspects, T-cell activation comprises cytokine signaling activation. In some aspects, after treatment, the proportion of activated T cells in the population is increased by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. In some aspects, the cytokine signaling activation is a IL2 receptor signaling activation. In some aspects, the cytokine signaling activation is a IL15 receptor signaling activation.

### Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Combination Therapies

The compositions of the disclosure comprising a) a first bispecific antibody comprising an antigen binding domain that binds to a first tumor associated antigen and an antigen binding domain that binds to a first subunit of a cytokine receptor; and b) a second bispecific antibody comprising an antigen binding domain that binds to a second tumor associated antigen and an antigen binding domain that binds to a second subunit of the cytokine receptor, is administered in combination with any other standard therapy; such methods are known to the skilled artisan and described in Remington's Pharmaceutical Sciences by E. W. Martin. If desired, the compositions of the disclosure is administered in combination with any conventional anti-neoplastic therapy, including but not limited to, immunotherapy, therapeutic antibodies, targeted therapy, surgery, radiation therapy, or chemotherapy.

In some aspects, the composition of the disclosure is administered in combination with c) a third bispecific antibody comprising an antigen binding domain that binds to a third tumor associated antigen and an antigen binding domain that binds to an antigen expressed on a T-cell. In some aspects, the antigen expressed on a T-cell is a CD3 antigen. Without being bound by theory, addition of a third bispecific antibody could improve the clinical outcome of the composition by further activating T cell subpopulations using bispecific engagers that target a TAA and a TCR complex. Without being bound by theory the inventors believe that combining the bispecific antibodies of the disclosure can stimulate both a primary signaling pathway that promotes T-cell mediated lysis of tumor cells (by clustering TCRs, for example) and a second co-stimulatory pathway to induce T-cell proliferation to overcome anergy.

### Definitions

As used herein, including the appended claims, the singular forms of words such as “a,” “an,” and “the,” include their corresponding plural references unless the context clearly dictates otherwise.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K.sub.D). Affinity can be measured by common methods known in the art, including KinExA and Biacore.

As used herein, the term “antibody” includes, but is not limited to, monoclonal antibodies, polyclonal antibodies, multispecific antibodies (e.g., bispecific antibodies), fully human antibodies, and chimeric antibodies.

As used herein, unless otherwise indicated, “antigen-binding fragment” refers to antigen-binding fragments of antibodies, i.e. antibody fragments that retain the ability to bind to the antigen bound by the full-length antibody, e.g. fragments that retain one or more CDR regions. Examples of antibody binding fragments include, but are not limited to, Fab, Fab′, F(ab′).sub.2, Fv fragments and individual antibody heavy chains or light chains, and individual heavy chain or light chain variable regions.

A “Fab fragment” is comprised of one light chain and the CH1 and variable regions of one heavy chain. The heavy chain of a Fab molecule cannot form a disulfide bond with another heavy chain molecule. A “Fab fragment” can be the product of papain cleavage of an antibody.

A “Fc” region contains two heavy chain fragments comprising the CH1 and CH2 domains of an antibody. The two heavy chain fragments are held together by two or more disulfide bonds and by hydrophobic interactions of the CH3 domains.

A “Fab′ fragment” contains one light chain and a portion or fragment of one heavy chain that contains the VH domain and the CH1 domain and also the region between the CH1 and CH2 domains, such that an interchain disulfide bond can be formed between the two heavy chains of two Fab′ fragments to form a F(ab′).sub.2 molecule.

A “F(ab′)2 fragment” contains two light chains and two heavy chains containing a portion of the constant region between the CH1 and CH2 domains, such that an interchain disulfide bond is formed between the two heavy chains. A F(ab′).sub.2 fragment thus is composed of two Fab′ fragments that are held together by a disulfide bond between the two heavy chains. An “F(ab′)2 fragment” can be the product of pepsin cleavage of an antibody. The “Fv region” comprises the variable regions from both the heavy and light chains but lacks the constant regions.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic “antibody” structural unit comprises a tetramer. In a monospecific antibody, each tetramer includes two identical pairs of polypeptide chains, each pair having one “light” (about 25 kDa) and one “heavy” chain (about 50-70 kDa). The amino-terminal portion of each chain includes a “variable region” or “variable domain” of about 100 to 110 or more amino acids primarily responsible for antigen recognition. The carboxy-terminal portion of the heavy chain may define a constant region primarily responsible for effector function.

Typically, human constant light chains are classified as kappa and lambda light chains. Furthermore, human constant heavy chains are typically classified as mu, delta, gamma, alpha, or epsilon, and define the antibody's isotype as IgM, IgD, IgG, IgA, and IgE, respectively. Subtypes of these IgG include, for example, IgG1 and IgG4.

“Variable region,” “variable domain,” “V region,” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V.sub.H.” The variable region of the light chain may be referred to as “V.sub.L.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V.sub.H.beta-sheet framework, or one of three hypervariable regions (L1, L2, or L3) within the non-framework region of the antibody V.sub.L.beta.-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved .beta.-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

Sequence identity refers to the degree to which the amino acids of two polypeptides are the same at equivalent positions when the two sequences are optimally aligned.

Sequence similarity includes identical residues and non-identical, biochemically related amino acids. Biochemically related amino acids that share similar properties and may be interchangeable are discussed above.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

The term “epitope,” as used herein, refers to an area or region on an antigen to which an antibody or antigen-binding fragment binds. Binding of an antibody or antigen-binding fragment thereof disclosed herein to an epitope means that the antibody or antigen-binding fragment thereof binds to one or more amino acid residues within the epitope.

“Isolated” nucleic acid molecule or polynucleotide means a DNA or RNA, e.g., of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a polynucleotide comprising” (or the like) a particular nucleotide sequence does not encompass intact chromosomes. Isolated polynucleotides “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to polynucleotide sequences necessary or helpful for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers. In an embodiment of the invention, the polynucleotide is operably linked to a promoter such as a viral promoter, a CMV promoter, an SV40 promoter or a non-viral promoter or an elongation factor (EF)-1 promotor; and/or an intron.

A nucleic acid is “operably linked” when it is placed into a functional relationship with another polynucleotide. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the polynucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

Host cells include eukaryotic and prokaryotic host cells, including mammalian cells. Host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells and HEK-293 cells. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Pichia finlandica, Pichia trehalophia, Pichia koclamae, Pichia membranaefaciens, Pichia minuta (Ogataea minuta, Pichia lindnen), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. The present invention includes any host cell (e.g., a CHO cell or Pichia cell, e.g., Pichia pastoris) containing an anti-ILT4 antibody or antigen-binding fragment thereof or containing a polynucleotide encoding such an antibody or fragment or containing a vector that contains the polynucleotide.

“Treat” or “treating” means to administer antibodies or antigen-binding fragments thereof of the present invention, to a subject having one or more symptoms of a disease for which the antibodies and antigen-binding fragments are effective, e.g., in the treatment of a subject having cancer or an infectious disease, or being suspected of having cancer or infectious disease, for which the agent has therapeutic activity. Typically, the antibody or fragment is administered in an “effective amount” or “effective dose” which will alleviate one or more symptoms (e.g., of cancer or infectious disease) in the treated subject or population, whether by inducing the regression or elimination of such symptoms or by inhibiting the progression of such symptom(s), e.g., cancer symptoms such as tumor growth or metastasis, by any clinically measurable degree. The effective amount of the antibody or fragment may vary according to factors such as the disease stage, age, and weight of the patient, and the ability of the drug to elicit a desired response in the subject.

### EXAMPLES

### Example 1: Construction and Production of KiH Bispecific Antibodies

1.1 BsAb construction. Complementarity determining regions (CDRs) of anti-IL-2Rγ (P1A3), anti-IL-2Rβ (P2C4) and anti-HER2 (Pertuzumab and Trastuzumab) were obtained from published sequences (WO 2017/021540 A1 and US 2006/0275305 A1 respectively) and used for proof of concept. The CDR sequences were synthesized by Eurofins and engineered into IL-2R×HER2 or IL-2Rβ×IL-2Rγ bsAbs using the Knob-in-the-Hole (KiH), CrossMAb technologies (US 2017/0129962 A1) with an inactive Fc (LALA PA mutations: leucine 234, leucine 235 and proline 329 were substituted with alanine residues). In all IL-2R×HER2 bsAb constructs, CDR sequences for anti-HER2 arms (both Pertuzumab and Trastuzumab) were inserted into the knob arm. In the P1A3× P2C4 construction, the CDR sequence of anti-IL-2Rβ was inserted into the knob arm. The gene fragments were cloned through restriction enzymes (BsrGI and PshAI, XbaI and EcoRI, AscI and BamHI, AfeI and Cla I) into an expression vector under the control of the hCMV promoter. After each cloning step, the newly engineered plasmid was transfected into XL1 bacteria for amplification and selection using ampicillin resistance. After plasmid purification, the desired constructions were verified by sequencing.

1.2 Expression and purification. Plasmids were transfected transiently into PEAK cells using Lipofectamine 2000 Transfection Reagent (Invitrogen, #11668-019). BsAbs were purified from the supernatant 5-6 days after transfection using CaptureSelect™ FcXL Affinity Matrix (Thermo Fisher, #194328050) and CaptureSelect™ IgG-CH1 Affinity Matrix (Thermo Fisher, #194320005) successively and were eluted using glycine pH 3.0 buffer and desalted against 25 mM Histidine/125 mM NaCl pH 6.0 buffer in 50 kDa Amicon Ultra Centrifugal filter units (#ACS505024). The final bsAb product was quantified using the Nanodrop and its quality was verified by SEC-HPLC, electrophoresis in denaturing conditions, IsoElectric Focusing gel and by Agilent 2100 Bionalayzer with the Protein 80 kit.

### Example 2: Phage Display Selection & Screening of IL2-Rb or IL2-Rg Fvs Using Human scFv Libraries Containing a Fixed Variable Heavy Chain Domain

General procedures for construction and handling of human scFv libraries displayed on M13 bacteriophage are described in Vaughan et al., [20], hereby incorporated by reference in its entirety. The libraries for selection and screening encode scFv that all share the same VH domain and are solely diversified in the VL domain. Methods for the generation of fixed VH libraries and their use for the identification and assembly of bispecific antibodies are described in US 2012/0184716 and WO 2012/023053, each of which is hereby incorporated by reference in its entirety. The procedures to identify scFv binding to human IL-2Rb or IL-2Rg are described below. Selections were performed with biotinylated human IL-2Rb or IL-2Rg proteins pre-coated on magnetic beads. Selection strategies included up to 4 rounds of selections.

2.1 Protein selections: Aliquots of scFv phage libraries were blocked with PBS containing 2% (w/v) skimmed milk. Blocked phages were first deselected on streptavidin/neutravidin magnetic beads (Dynabeads™ M-280 Streptavidin Magnetic Beads or Sera-Mag SpeedBeads Neutravidin™ Coated Magnetic Particles) then incubated with 100 nM, 10 nM or 1 nM of biotinylated recombinant human and cynomolgus IL2Rb or IL2Rg (commercial or in house produced) pre-captured on the same kind of beads used for the deselection. The mix was washed five times with PBS/1% BSA/0.1% Tween® 20 and twice with PBS only. Phages were eluted with 1 mg/mL Trypsin and, after the addition of AEBSF to block trypsin activity, the eluate was directly added to exponentially growing TG1 bacteria cells. Outputs were rescued and used for the next round of selection.

2.2 Screening for scFv binding/non-binding to human IL-2R: Screening of scFv for binding to IL-2Rb or IL-2Rg was tested by ELISA using biotinylated recombinant proteins (and biotinylated huMSLN protein as irrelevant control).

2.3. ELISA: For the binding ELISA, neutravidin-coated plates were blocked with 1% casein in PBS. Biotinylated IL-2Rb, IL-2Rg and huMSLN (Mesothelin) were captured at 5 nM. Dilution of freshly prepared periplasmic extracts containing the selected scFv were applied to the plates and detected using a combination of mouse anti-c-myc antibody and donkey anti-mouse IgG-HRP antibody. Following the addition of TMB the OD at 450 nm was measured using a spectrophotometer plate reader. Hits were classified as specific binders if unable to bind to the irrelevant huMSLN protein and if the OD₄₅₀ on IL-2Rb or IL-2Rg was at least 3 times higher than the background OD₄₅₀. Hits were sequenced following DNA extraction from single clones.

### Example 3: Fixed VH Candidates Reformatting into IgG and Transient Expression in Mammalian Cells

After screening and sequencing, scFv candidates with the desired binding properties were reformatted into IgG and expressed by transient transfection into PEAK cells. The VH and VL sequences of selected scFv were amplified with specific oligonucleotides and cloned into an expression vector containing the heavy and light chain constant regions. The expression vectors were verified by sequencing and transfected into mammalian cells using Lipofectamine 2000 (Thermo Fisher Scientific) according to manufacturer's instructions. Briefly, 4×10⁶ PEAK cells were cultured in T75 flasks in 25 ml culture media containing fetal bovine serum. Transfected cells were cultured for 5-6 days at 37° C., IgG production was quantified using an Octet RED96 instrument. The supernatant was harvested for IgG purification on FcXL affinity resin (Thermo Fisher Scientific) according to manufacturer's instructions. Briefly, supernatants from transfected cells were incubated overnight at 4° C. with an appropriate amount of FcXL resin. After resin wash with PBS, samples were loaded on Amicon Pro column and the IgG consequently eluted in 50 mM Glycine pH 3.5. The eluted IgG fraction was then dialyzed by Amicon 50 kDa against Histidine NaCl pH 6.0 buffer and the IgG content is quantified by absorption at 280 nm. Purity and IgG integrity were verified by electrophoresis using an Agilent Bioanalyzer 2100 according to manufacturer instructions (Agilent Technologies).

### Example 4: Expression and Purification of Bispecific Antibodies Carrying a Lambda and a Kappa Light Chain

The simultaneous expression of one heavy chain and two lights chain in the same cell can lead to the assembly of three different antibodies. Simultaneous expression can be achieved in different ways such as that the transfection of multiple vectors expressing one of the chains to be co-expressed or by using vectors that drive multiple gene expression.

Here, the two light chains were cloned into the vector pNovi κHλ that was previously generated to allow for the co-expression of one heavy chain, one Kappa light chain and one Lambda light chain as described in US20120184716 and WO2012023053, each of which is hereby incorporated by reference in its entirety. The expression of the three genes is driven by human cytomegalovirus promoters (hCMV) and the vector also contains a glutamine synthetase gene (GS) that enables the selection and establishment of stable cell lines. The common VH and the VL genes of IL-2Rb or IL-2Rg IgG (both described in the present patent) and of the anti-MSLN IgG (O30, O35, O38, O41, according to the patent WO2018215835A1) or of the anti-HER2 IgG (N2-19, N2-28, described in the present patent) were cloned in the vector pNovi κHλ, for transient expression in mammalian cells. All constructs were generated based on an inactive Fc referred as /N in the document (LALA PA mutations: leucine 234, leucine 235 and proline 329 were substituted with alanine residues). Expi293 cells were cultured in suspension in an appropriate Erlenmeyer flask with suitable number of cells and culture medium volume. Plasmid DNA was transfected into Expi293 cells using PEI. Antibody concentration in the supernatant of transfected cells was measured during the production using an Octet RED96. According to antibody concentration, supernatants were harvested 5 to 7 days after transfection and clarified by filtration after addition of diatomaceous earth (Sartorius). The purification was based on a three-step purification process. First, the CaptureSelect™ FcXL affinity matrix (Thermo Fisher Scientific) was washed with PBS and then added in the clarified supernatant. After incubation overnight at +4° C. and 20 rpm, supernatants were centrifuged at 2000 g for 10 min, flow through was stored and resin were washed twice with PBS. Then, the resin was transferred on Amicon Pro columns and a solution containing 50 mM glycine at pH 3.5 was used for elution. Several elution fractions were generated, neutralized with Tris-HCl pH7.4 and pooled. The pool containing total human IgGs (the bispecific and the two monospecific antibodies) was quantified using a Nanodrop spectrophotometer (NanoDrop Technologies). A small aliquot was stored for further analysis and the remaining sample was incubated for 30 min at RT and 20 rpm with the appropriate volume of CaptureSelect™ KappaXL affinity matrix (Thermo Fisher Scientific). Resin recovery and wash, elution and neutralization steps were performed as described above. The last affinity purification step was performed using the CaptureSelect™ lambda Fab affinity matrix (Thermo Fisher Scientific) applying the same process as for the kappa purification step. Alternatively, the purification was based on a two-step purification process, where only the CaptureSelect™ KappaXL affinity matrix and the CaptureSelect™ lambda Fab affinity matrix were used. All elution fractions were pooled and desalted against His-NaCl pH 6.0 formulation buffer using 50 kDa Amicon Ultra centrifugal filter units (Merck Millipore). The final product was quantified using the Nanodrop.

Purified bispecific antibodies were analyzed by electrophoresis in denaturing and reducing conditions using an Agilent 2100 Bioanalyzer with the Protein 80 kit as described by the manufacturer (Agilent Technologies). The aggregate level was determined by SEC-UPLC. All samples were tested for endotoxin contamination using the Limulus Amebocyte Lysate test (LAL; Charles River Laboratories).

### Example 5: Assessment of the Binding of the bsAbs to their Targets by ELISA

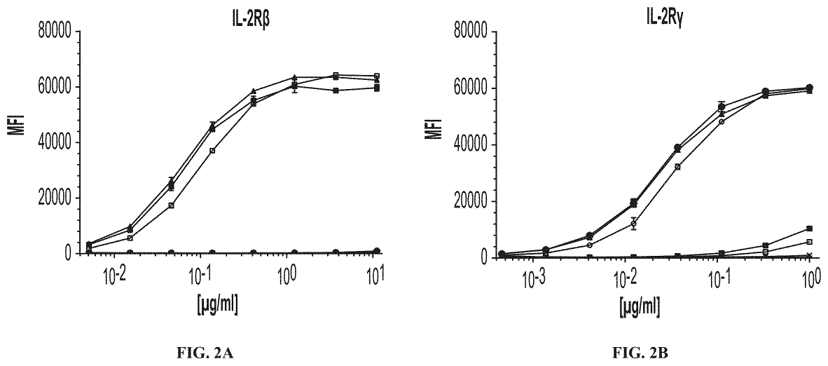

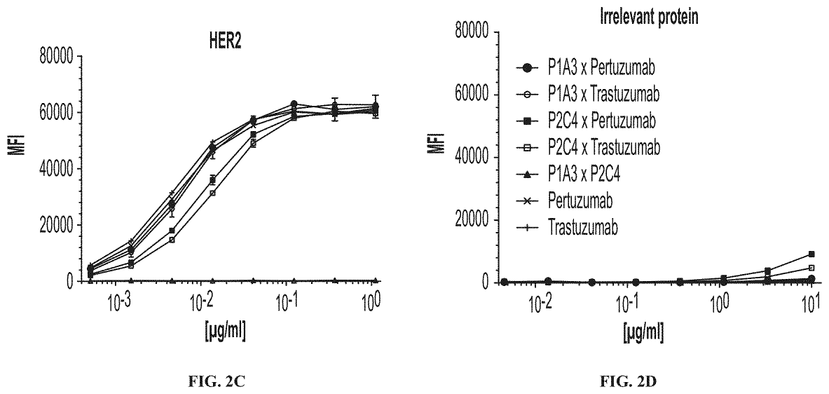

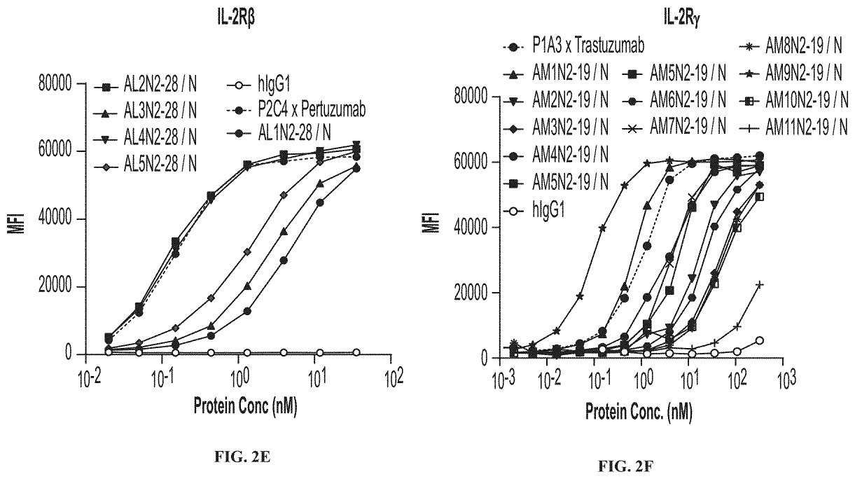

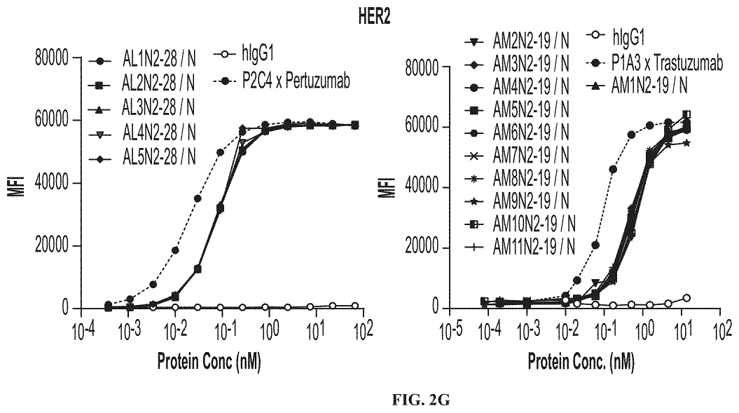

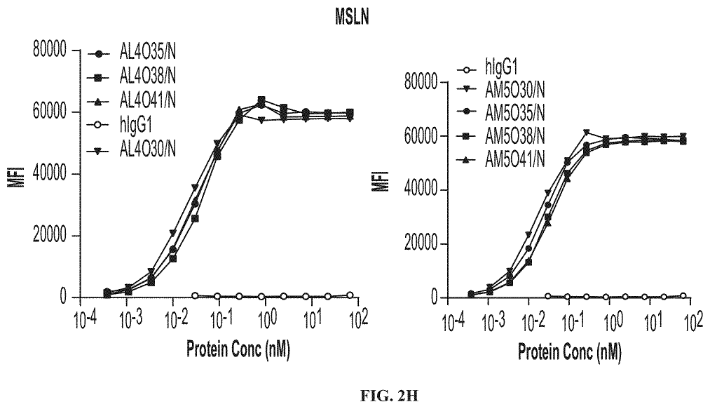

Binding specificity of all IL-2R×TAA bsAbs was evaluated by direct ELISA (FIGS. 2A-2H). Fifty μl/well of biotinylated human recombinant proteins IL-2Rβ, IL-2Rγ, HER2, MSLN and an irrelevant protein (recombinant Human carcinoembryonic antigen) were added onto streptavidin-coated 96-well black plates (Greiner, #655997) at a concentration of 1 μg/ml (concentration was adjusted to compensate for incomplete biotinylation). Binding of bsAb to coated proteins was assessed using HRP-linked goat anti-human IgG-Fcγ (#109-036-098) and Amplex™ UltraRed Reagent (#A36006). Data were acquired using a Synergy HT (Biotek) at the following wavelengths: excitation: 530/25, emission: 590/35. Binding to IL-2Rβ was confirmed for the KiH bsAbs carrying a P2C4 arm, i.e., P2C4× Pertuzumab, P2C4× Trastuzumab and P1A3×P2C4 (FIG. 2A); binding to IL-2Rγ was confirmed for the KiH bsAbs carrying a P1A3 arm, i.e., P1A3× Pertuzumab, P1A3× Trastuzumab and P1A3×P2C4 (FIG. 2B); while binding to HER2 was confirmed for all the bsAbs carrying an anti-HER2 arm, i.e., all bsAbs except P1A3×P2C4 (FIG. 2C). None of the bsAbs binds to the irrelevant protein (FIG. 2D).

IL-2R κλ-bodies were also generated targeting two different TAA: HER2 and MSLN. A first set of IL-2R×HER2 κλ-bodies were generated by pairing the same HER2 arm (N2-28) to different IL-2Rb arms (AL1 to AL5) and another HER2 arm (N2-19) to different IL-2Rg arms (AM1 to AM11). Another set of IL-2R×MSLN κλ-bodies were generated by pairing one IL-2Rb arm (AL4) and one IL-2Rg arm (AM5) to different MSLN arms (O30, O35, O38, O41, according to the patent WO2018215835A1). Similar ELISA assays were conducted to validate the binding to their respective targets. Binding to IL2Rb was confirmed for all κλ-bodies carrying an IL-2Rb arm (AL1 to AL5) (FIG. 2E). Binding to IL2Rg was confirmed for all κλ-bodies carrying an IL2Rg arm (AM1 to AM11) (FIG. 2F). Binding to HER2 was demonstrated for all IL-2R×HER2 κλ-bodies (FIG. 2G) and to MSLN for all IL-2R×MSLN κλ-bodies (FIG. 2H).

### Example 6: Assessment of Co-Engagement of IL-2β×HER2 and IL-2Rγ×HER2 KiH bsAbs to their Targets by Bio-Layer Interferometry

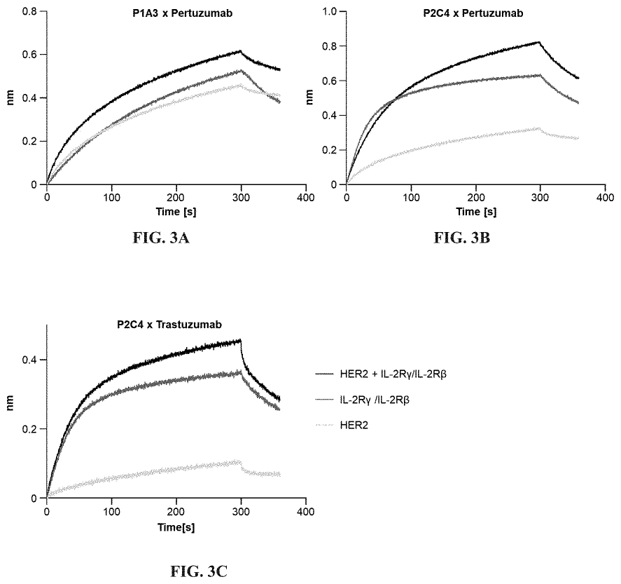

Binding to their targets by KiH bsAbs was assessed using Bio-Layer Interferometry (BLI) technology (OctetRED96). BsAbs were diluted at a concentration of 10 μg/ml in kinetic buffer (Sartorius, #18-1105) loaded onto Protein A biosensors (Sartorius, #18-5010). Biosensors were incubated in wells containing either an individual target or the two targets of the corresponding bsAb simultaneously. The results demonstrated that P1A3× Pertuzumab bound to both IL-2Rγ and HER2 separately and simultaneously (FIG. 3A), while P2C4× Pertuzumab and P2C4× Trastuzumab bound to IL-2Rβ and HER2 separately and simultaneously (FIGS. 3B and 3C). In consistency with the ELISA results, the BLI experiments confirmed the binding specificity of the bsAbs to their targets. Furthermore, the higher signal (wavelength shifts) obtained for P1A3× Pertuzumab, P2C4× Pertuzumab and P2C4× Trastuzumab bsAbs when both targets were present at the same time comparing to the condition with a single target evinces their ability to bind both targets simultaneously (FIGS. 3A-3C).

### Example 7: Assessment of IL-2 and IL-15 Signaling Neutralization by IL-2R×TAA bsAbs

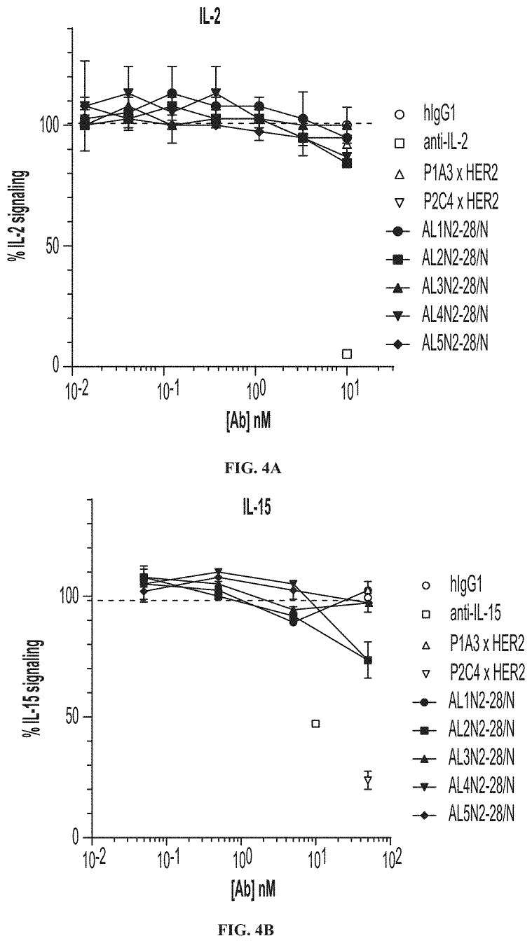

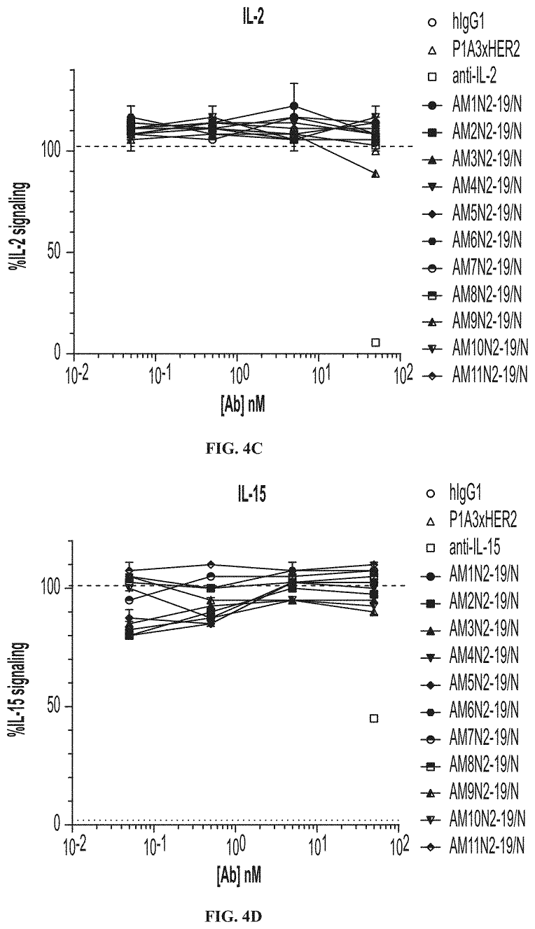

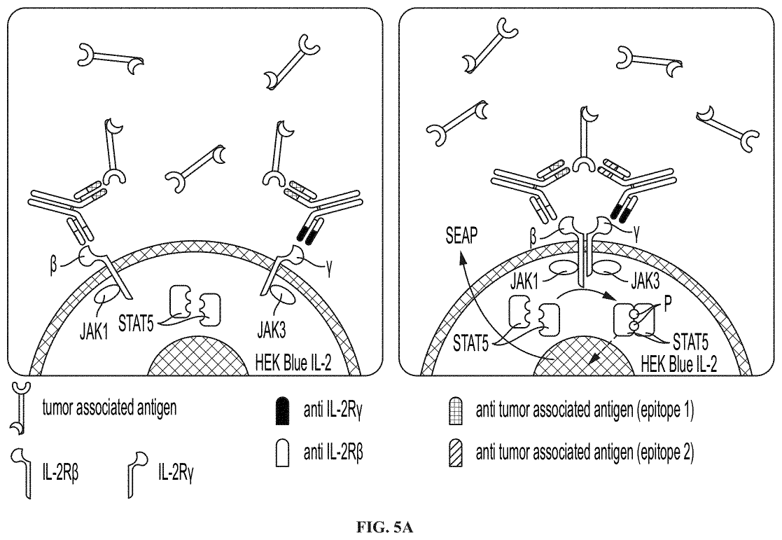

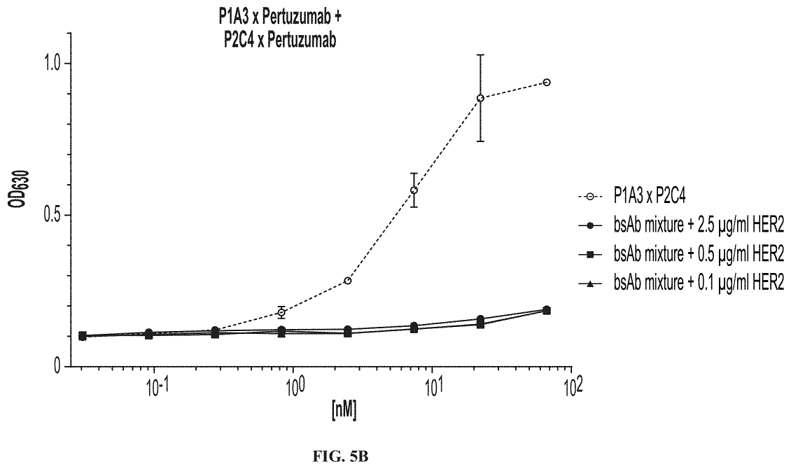

Once the binding of each IL-2R×TAA bsAbs of the present invention was confirmed, experiments were conducted to determine whether engaging IL-2Rb or IL-2Rg would interfere with native IL-2 or IL-15 signaling. For this purpose, IL-2-reporter cell line (expressing the dimeric IL-2R and responding both to IL-2 and IL-15) was stimulated with a fixed dose of IL-2 (0.2 or 1 nM, FIGS. 4A and 4C, respectively) or IL15 (0.003 nM, FIGS. 4B and 4D) in presence of a dose range of IL-2Rb×HER2 bsAbs (FIGS. 4A and 4B), or IL-2Rg×HER2 bsAbs (FIGS. 4C and 4D).

7.1. Cell culture: HEK Blue IL-2 cells (Invivogen, #hkb-il2bg) expressing IL-2Rb and IL-2Rg were used as a reporter cell line for IL-2 and IL-15 signaling assays. The cell line is designed to express the secreted embryonic alkaline phosphatase (SEAP) downstream of IL-2 signaling (SEAP expression is controlled by a pSTAT5 inducible promoter). Reporter cells were cultured at 37° C. in 5% CO2 in DMEM 4.5 g/l glucose 2 mM L-glutamine (#11965-084), fetal bovine serum (10% v/v, #F7524), gentamycin (25 μg/ml, #G1397-10ML), Normocin (100 μg/ml, #ant-nr-2), 1×HEK-Blue CLR-Selection (#hb-csm) and puromycin (1 μg/ml, #ant-pr-1).

7.2. IL-2 reporter cell assay: Assays were performed by incubating cells (HEK blue IL-2Rb/g) in flat-bottom 96-well plates (100000 cells/well) under different test conditions in DMEM 4.5 g/l glucose 2 mM L-glutamine (#11965-084), fetal bovine serum (10% v/v, #F7524), gentamycin (25 μg/ml, #G1397-10ML) at 37° C. in 5% CO2 for 20-24 hours. SEAP levels were measured by incubating 60 min, 20 μl of supernatant from each well with 180 μl Quanti Blue™ Solution (Invivogen, #rep-qbs). at 37° C. in clear bottom 96-well plates. The absorbance at 630 nm was acquired using a Synergy HT (Biotek).

The results demonstrated that, in the dose range tested, IL-2Rb×HER2 bsAbs (FIGS. 4A and 4B) or IL-2Rg×HER2 bsAbs (FIGS. 4C and 4D) do not interfere with IL-2 signaling (FIGS. 4A and 4B) nor with IL-15 signaling (FIGS. 4B and 4D).

### Example 8: Activation of IL-2 Signaling by the Combination of IL-2Rβ×HER2 and IL-2Rγ×HER2 KiH bsAbs in the Presence of Soluble HER2

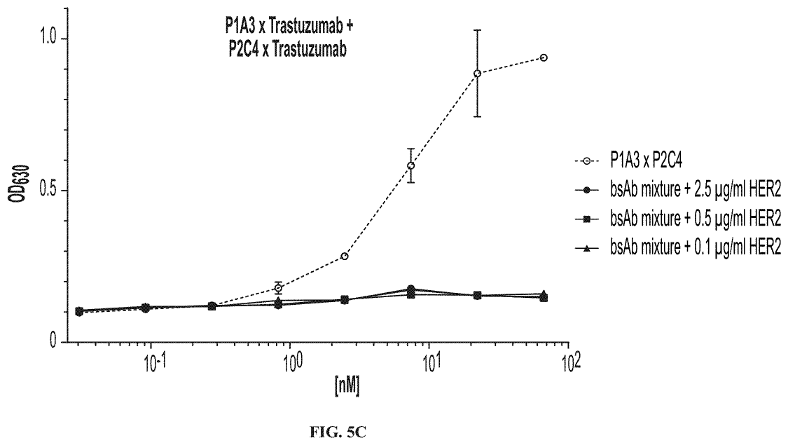

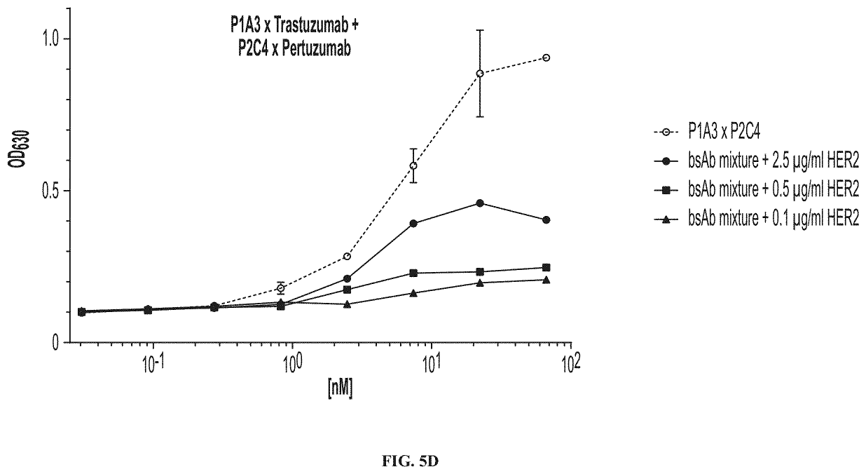

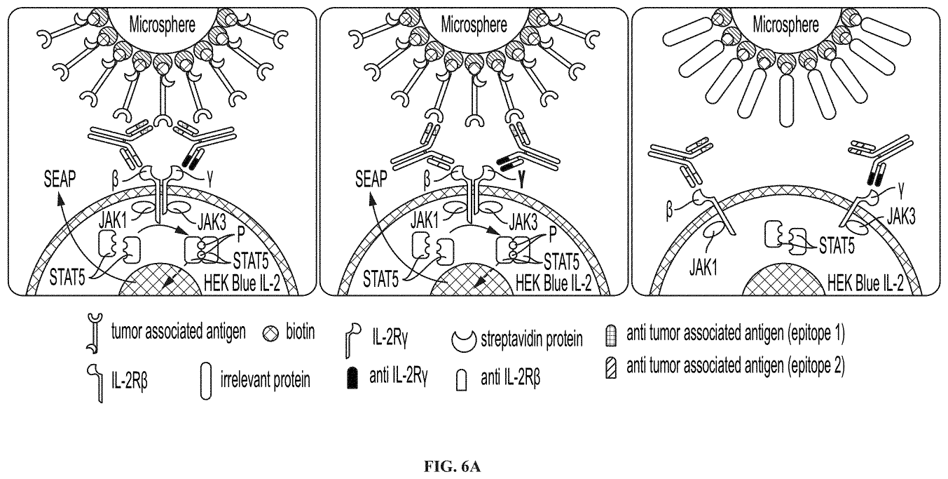

To demonstrate IL-2-agonistic activity of IL-2R×HER2 bsAb combination in presence of a soluble TAA (e.g. HER2), experiments were conducted where soluble HER2 was mixed with a dose range of IL-2Rb×HER2+IL-2Rg×HER2 KiH bsAb combination (FIG. 5A). As expected, incubation of P1A3×P2C4 KiH bsAb with reporter cell line induced IL-2 signaling (FIGS. 5B and 5D). However, the combination of two IL-2R×HER2 bsAbs did not induce IL-2 signaling in the absence of HER2 (data not shown). In the presence of soluble HER2, the combination of two IL-2R×HER2 bsAbs targeting the same epitope on HER2 (P1A3× Pertuzumab+P2C4× Pertuzumab or P1A3× Trastuzumab+P2C4× Trastuzumab) did not induce IL-2 signaling (FIGS. 5B and 5C). In contrast, the combination of two IL-2R×HER2 bsAbs targeting two different epitopes on HER2 (P1A3× Trastuzumab+P2C4× Pertuzumab) induced IL-2 signaling when incubated with soluble HER2 (FIG. 5D). Furthermore, activation of the pathway was dependent on soluble HER2 concentration.

Activation of IL-2 signaling requires IL-2Rβ (CD122) and IL-2Rγ (CD132) being brought into proximity of each other. As soluble HER2 monomers can only bring together two KiH bsAbs targeting two separate epitopes on HER2 (FIG. 5A), the results obtained with the combination of KiH bsAbs in the presence of soluble HER2 are in agreement with expectations.

### Example 9: Activation of IL-2 Signaling by the Combination of IL-2Rβ×TAA and IL-2Rγ×TAA bsAbs in the Presence of TAA-Coated Microspheres

9.1. Cell culture: HEK Blue IL-2 cells (Invivogen, #hkb-i12) expressing IL-2Ra, IL-2Rb and IL-2Rg were used as a reporter cell line for IL-2 signaling assays. The cell line is designed to express the secreted embryonic alkaline phosphatase (SEAP) downstream of IL-2 signaling (SEAP expression is controlled by a pSTAT5 inducible promoter). Reporter cells were cultured at 37° C. in 5% CO2 in DMEM 4.5 g/l glucose 2 mM L-glutamine (#11965-084), fetal bovine serum (10% v/v, #F7524), gentamycin (25 μg/ml, #G1397-10ML), Normocin (100 μg/ml, #ant-nr-2), 1×HEK-Blue CLR-Selection (#hb-csm) and puromycin (1 μg/ml, #ant-pr-1).

9.2. IL-2 reporter cell assay: Assays were performed by incubating streptavidin-biot-TAA-coated microsphere (300000 beads/well) and cells in flat-bottom 96-well plates (50000 cells/well) under different test conditions in DMEM 4.5 g/l glucose 2 mM L-glutamine (#11965-084), fetal bovine serum (10% v/v, #F7524), gentamicin (25 μg/ml, #G1397-10ML) at 37° C. in 5% CO₂ for 20-24 hours. SEAP levels were measured by incubating 20 μl of supernatant from each well with 180 μl Quanti Blue™ Solution (Invivogen, #rep-qbs) for 120 min. at 37° C. in clear bottom 96-well plates. The absorbance at 630 nm was acquired using a SpectraMax-i3x (Molecular Devices).

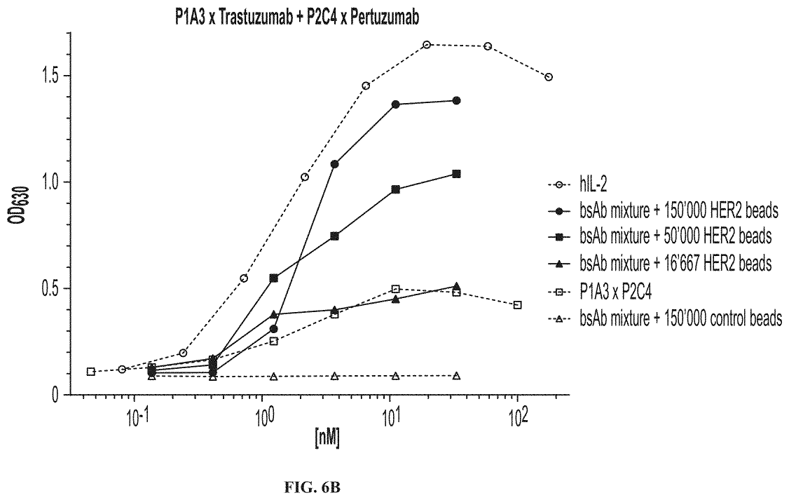

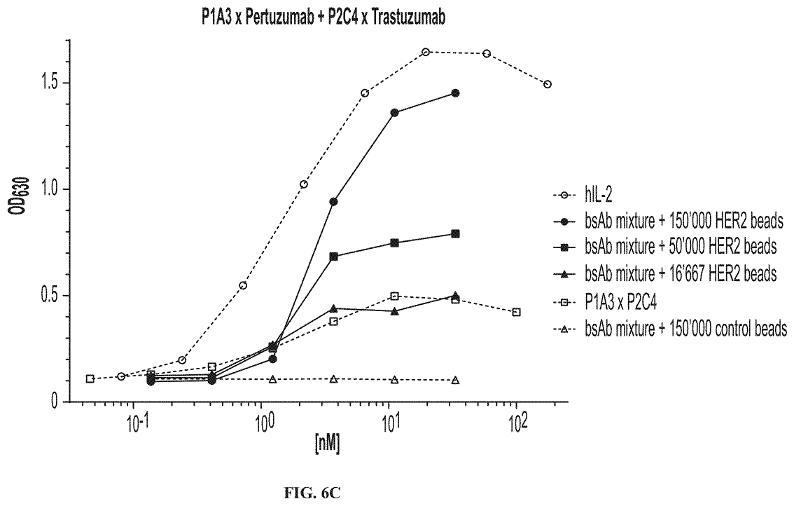

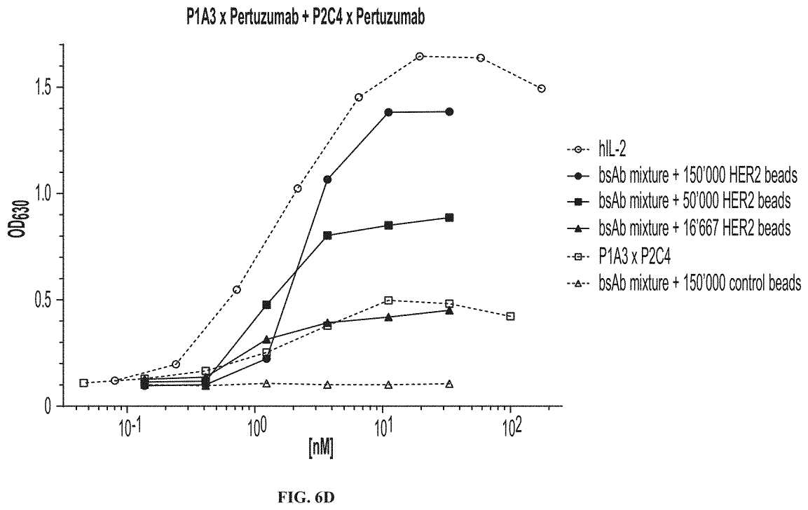

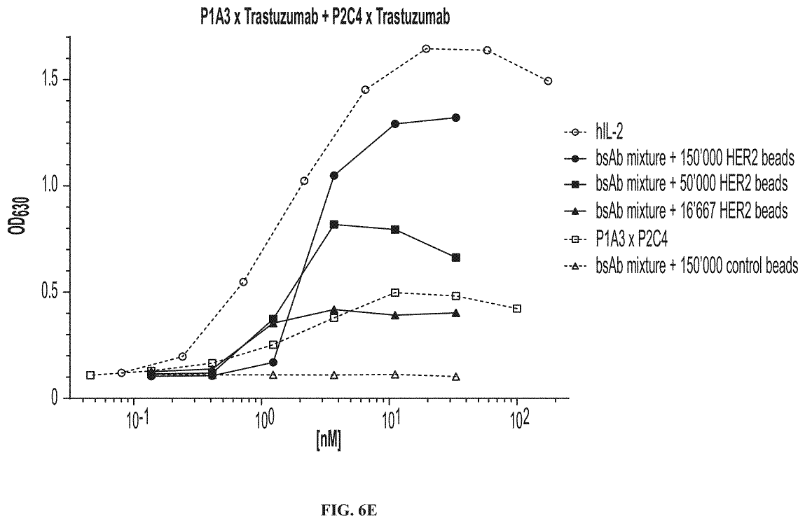

As shown in the previous example, in the presence of soluble HER2 monomers, only the combination of two IL-2R×HER2 bsAbs targeting two different epitopes on HER2 can induce IL-2 signaling. As clustering of HER2 on cell surface has the potential to bring two IL-2R×HER2 bsAbs targeting the same HER2 epitope into proximity of each other, it is expected that, in the presence of surface clustered HER2, the combination of two IL-2R×HER2 bsAbs targeting the same HER2 epitope can also induce IL-2 signaling. To test this hypothesis, HER2-coated microspheres (#24158-5) were used to mimic HER2 expressed on cell surface in the IL-2 reporter assay (FIG. 6A). In the presence of HER2-coated microspheres, both the combinations of two IL-2R×HER2 bsAbs targeting two different HER2 epitopes (FIGS. 6B and 6C) and the combinations targeting the same HER2 epitope (FIGS. 6D and 6E) induced IL-2 signaling activation. With both types of combinations, we observed a positive correlation between the level of IL-2 signaling measured and the concentrations of HER2-coated microspheres. Importantly the agonistic effect of the combination of two IL-2R×HER2 bsAbs was much stronger compared to what was observed with the P1A3×P2C4 bsAb (non-TAA targeted) and reached a level close to that induced by recombinant human IL-2 (FIGS. 6B-6E). Incubation of the bsAb combinations with high concentrations of microspheres coated with an irrelevant protein did not activate IL-2 signaling.

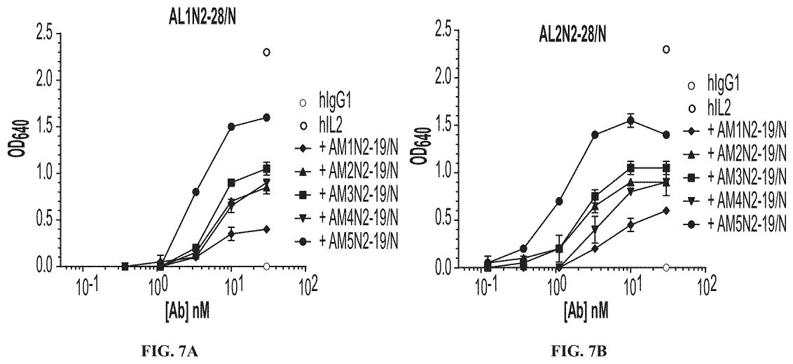

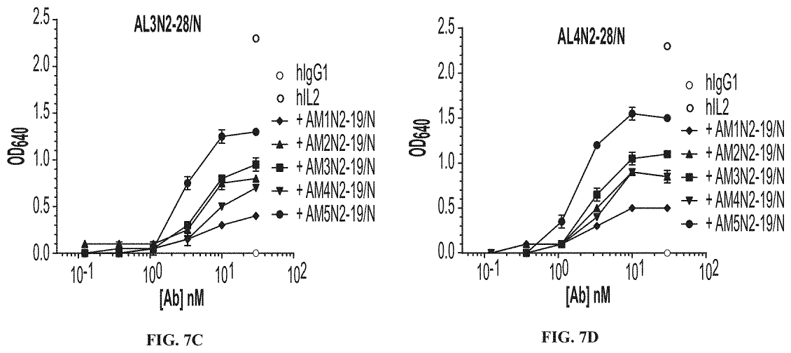

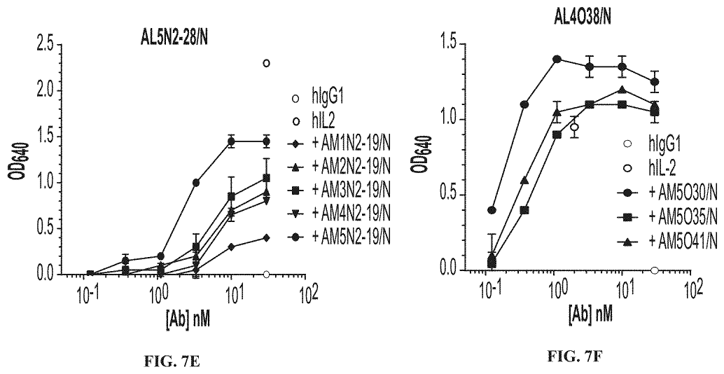

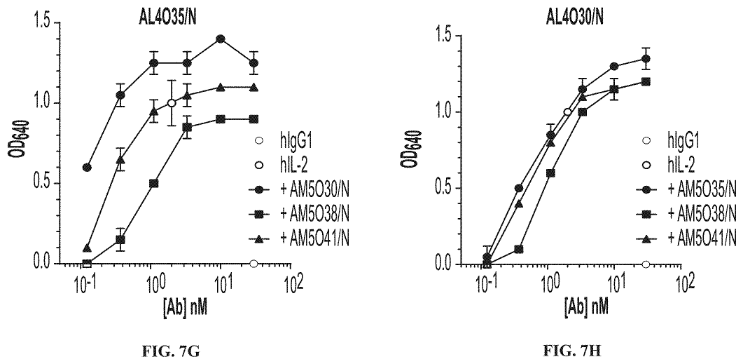

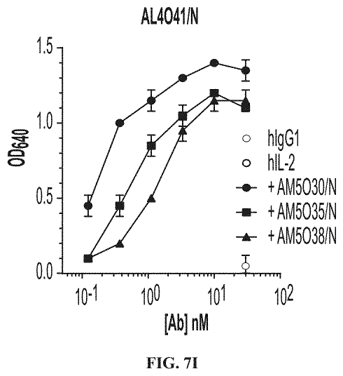

Similar findings were generated with various combinations of IL-2R×TAA bodies (FIGS. 7A-7I). A first set of IL-2R×HER2 bsAbs was tested by combining κλ-bodies sharing the same HER2 arm (N2-28) but paired with different IL-2Rb arms (AL1 to AL5) and κλ-bodies sharing the same HER2 arm (N2-19, targeting another HER2 epitope than N2-28) paired with different IL-2Rg arms (AM1 to AM5). All κλ-bodies were cross-combined and tested as depicted in FIG. 6A in presence of HER2-coated beads (fixed 6:1, beads: IL-2 reporter cell line ratio). Data showed potent IL-2 agonistic activity regardless of the combination tested (FIG. 7). Interestingly, IL-2 agonistic activity can be fine-tuned across different combinations. Higher IL-2 signaling can be induced when AL1N2-28/N is combined to AM52-29/N but is consistently weaker when combined to AM1N2-19/N (FIG. 7A). Similar data were generated by combining AM5N2-19/N to AL2N2-28/N (FIG. 7B), AL3N2-28/N (FIG. 7C), AL4N2-28/N (FIG. 7D) or AL5N2-28/N (FIG. 7E). The resulting IL-2 signaling can be modulated either by decreasing the concentration of the bsAb combination or by combining different pairs of IL-2R×HER2 κλ-bodies.

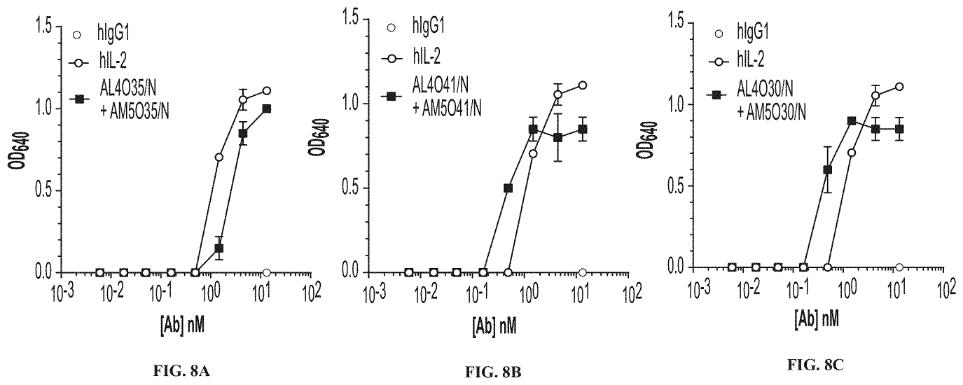

To demonstrate that IL-2R-bsAb combination can also mediate IL-2 signaling by engaging another TAA, an additional set of κλ-bodies was generated by pairing one IL-2Rb arm (AL4) and one IL-2Rg arm (AM5) to various MSLN targeting arms (O30, O35, O38, O41), each of them binding non-overlapping epitopes (patent WO2018215835A). Their activity in combination was tested using the same methodology as described above but replacing HER2-coated beads by MSLN-coated beads (FIG. 6A). All κλ-bodies were cross-combined and showed potent IL-2 agonistic activity regardless of the MSLN arms used (FIGS. 7F-7J). As observed for IL-2R×HER2 bsAbs, IL-2 agonistic activity can be fine-tuned across different combinations. Higher IL-2 signaling can be induced when AM5O30/N is combined to AL4O38/N (FIG. 7F). Similar data were generated by combining AM5O30/N to AL4O35 (FIG. 7G), AL4O30/N (FIG. 7H) or AL4O41/N (FIG. 7I). Importantly, as observed for IL-2R×HER2 bsAbs, the combinations of two IL-2R×MSLN bsAbs targeting the same MSLN epitope with O35 (FIG. 8A), O41 (FIG. 8B) or O30 (FIG. 8C) induced IL-2 signaling close to the level induced by recombinant human IL-2 (FIGS. 8A-8C).

### Example 10: Activation of IL-2 Signaling by the Combination of IL-2Rβ×TAA and IL-2Rγ×TAA bsAbs in the Presence of TAA Tumor Cells

The results described above showed that, in the presence of TAA-coated microspheres and regardless of the bsAb format used (KiH construct or κλ-body), the combination of IL-2Rb×TAA and IL-2Rg×TAA bsAbs can induce IL-2 signaling activation, suggesting that IL-2 agonistic activity could also be mediated in the presence of TAA expressing tumor cells. Both HER2 and MSLN expressing cell lines were used as a source of the TAA.

10.1. Tumor cell lines culture: Three HER2⁺ tumor cell lines were used: BT-474, SK-BR-3 and NCI-N87. BT-474 (ATCC, #HTB-20) is an invasive ductal carcinoma. SK-BR-3 (ATCC, #HBT-30) is a breast adenocarcinoma cell line. NCI-N87 (ATCC, #CRL-5822) is a gastric carcinoma cell line. HER2 is overexpressed in all 3 above mentioned tumor cell lines with BT-474, SK-BR-3 and NCI-N87 expressing 624′000, 587′000 and 552′000 HER2/cell respectively. As a source of MSLN, three tumor cell lines were used: NCI-H226, OVCAR-3 and NCI-N87. NCI-H226 (ATCC, #CRL-5826) is a lung squamous cell carcinoma. OVCAR-3 (ATCC, #HBT-161) is an epithelial ovarian carcinoma cell line. In contrast to HER2, MSLN is expressed at lower level with NCI-H226, OVCAR-3 and NCI-N87 cell lines expressing 180′000, 67′000 and 24′000 MSLN/cell respectively. These 5 adherent cell lines were cultured at 37° C. in 5% CO₂ and detached for subculturing using trypsin-EDTA solution. BT-474 were cultured in Hybri-care medium (ATCC, #46-X), fetal bovine serum (10% v/v, Thermo Fisher #F7524), L-glutamine (2 mM, Sigma, #G7513-100ML) and sodium bicarbonate (1.5 g/L, Sigma #58761-100 mL). SK-BR-3 were cultured in McCoy's 5A (Sigma, #M9309), 10% fetal bovine serum and L-glutamine. NCI-N87 were cultured in RPMI 1640 (Thermo Fisher, #11875-093), 10% fetal bovine serum and 2 mM L-glutamine.

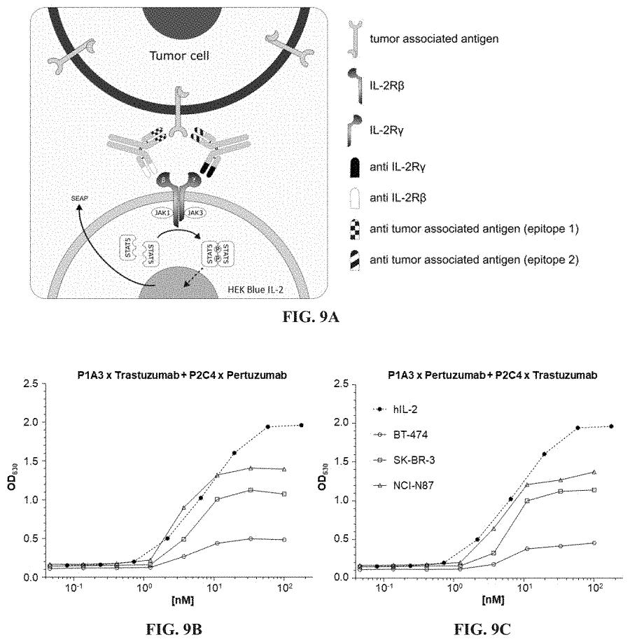

As shown in the previous example, in the presence of HER2-coated microspheres, the combination of two IL-2R×HER2 bsAbs can induce IL-2 signaling. It is expected that the combination of two IL-2R×HER2 bsAbs in the presence of HER2⁺ overexpressing tumor cells will also activate IL-2 signaling. To test this hypothesis, we first used the IL-2-reporter assay by replacing HER2-coated microspheres with HER2⁺ tumor cells and mixed 100′000 tumor cells to 100′000 HEK Blue IL-2 cells (FIG. 9A). In the presence of tumor cells overexpressing HER2+, both combinations of IL-2R×HER2 KiH bsAbs tested induced a dose-dependent IL-2 signaling activation (FIGS. 9B-9C) and was close to those induced by recombinant human IL-2 in the presence of NCI-N87. This observation confirms that our combination of IL-2R×HER2 bsAbs can activate IL-2 signaling in the presence of HER2 expressing cells.

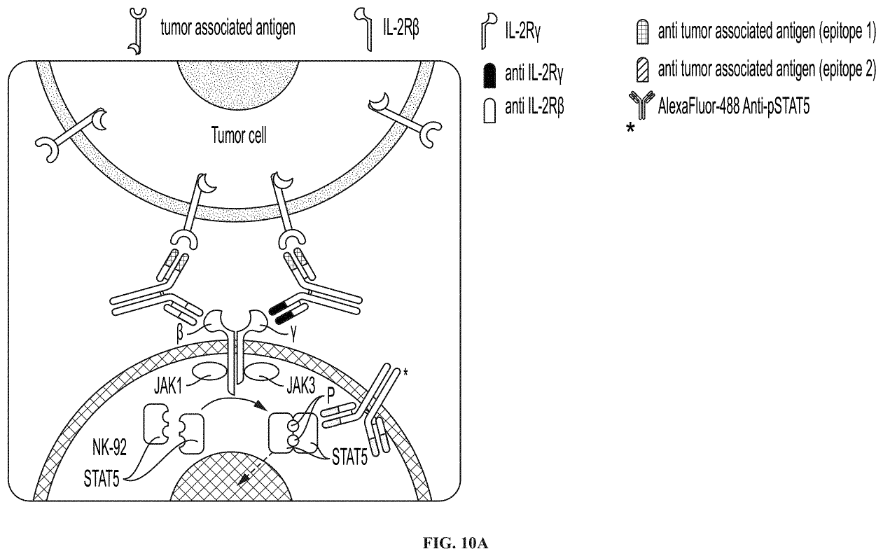

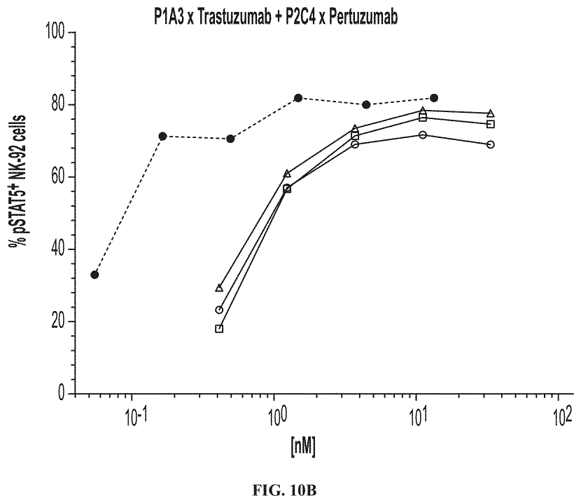

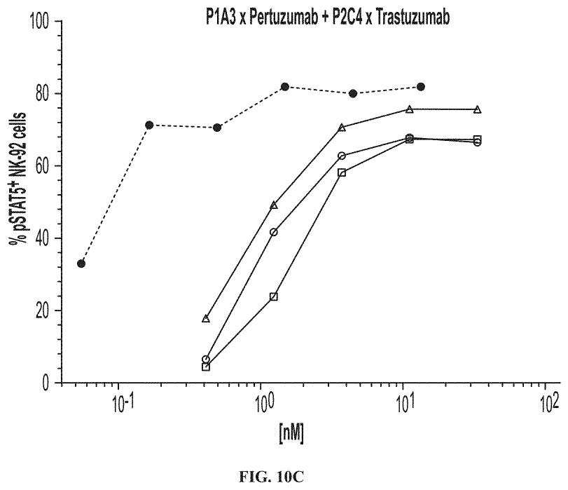

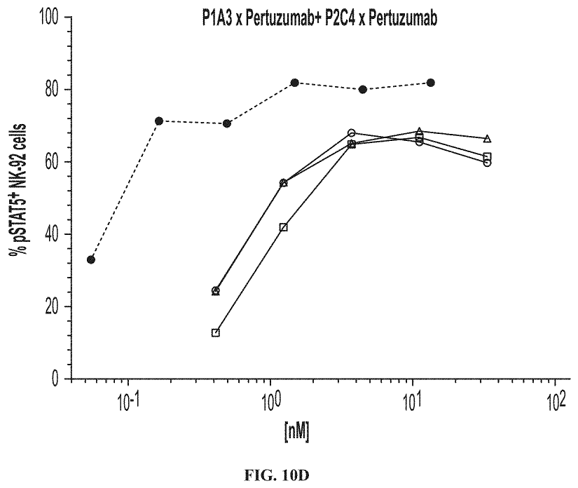

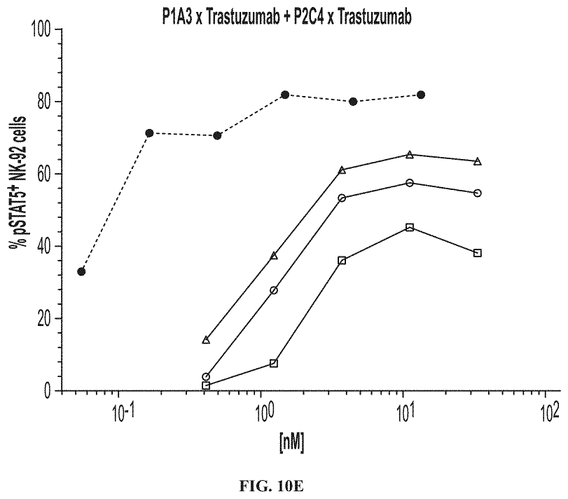

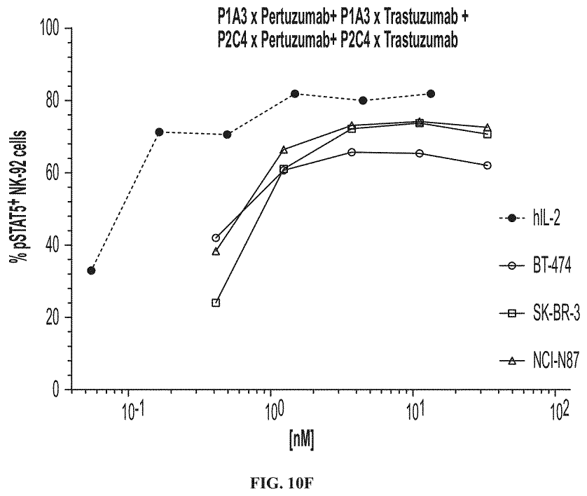

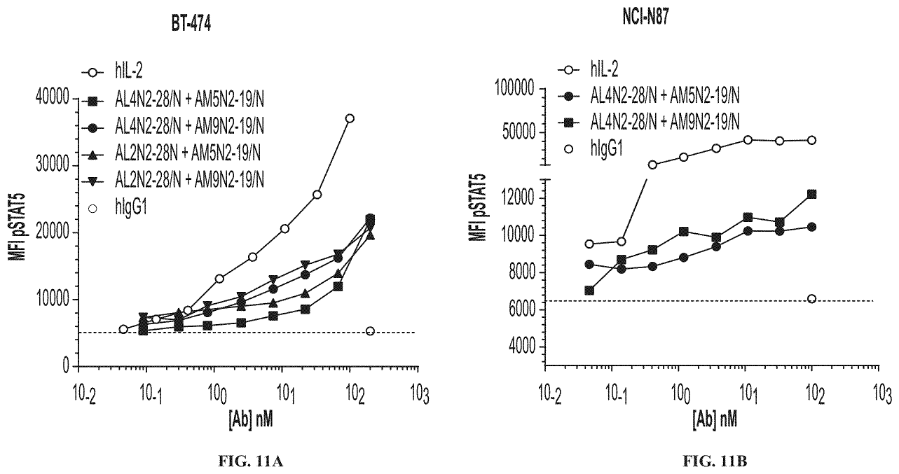

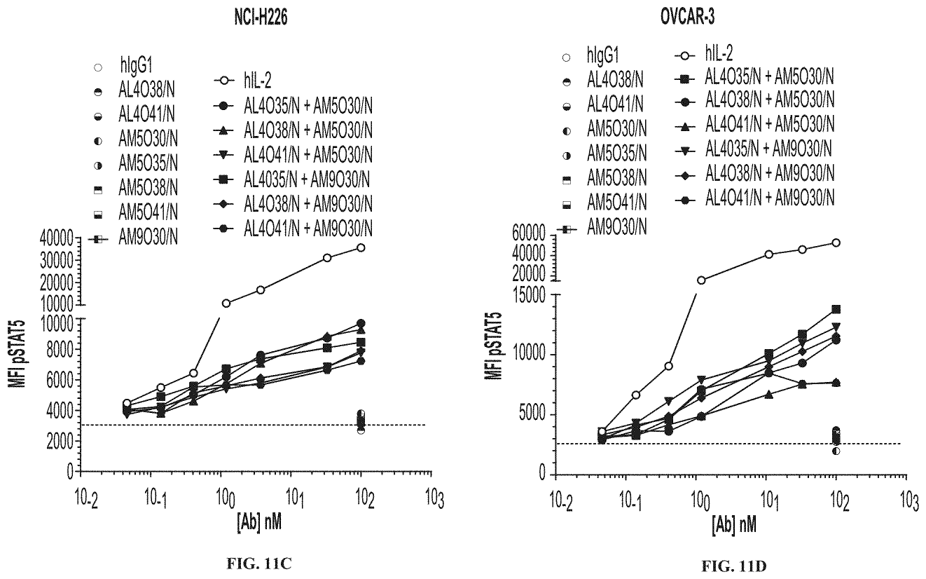

In the previous example we showed that IL-2 signaling can be induced in reporter cells in the presence of HER2 over-expressing tumor cells through our combination of IL-2R×HER2 bsAbs. To extend this observation to non-transduced cells of immune origin we chose NK-92 (DSMZ, #ACC 488), a natural killer lymphoma cell line. To quantify IL-2 signaling activation in NK-92 cells we measured the level of pSTAT5 by flow cytometry, which is a widely used method to evaluate IL-2 signaling activation. STATS is a transcription factor that is involved in the signaling pathway of IL-2. Upon IL-2 engagement on IL-2Rβ and IL-2Rγ, JAK1 and JAK3 are recruited and can phosphorylate STATS. pSTAT5 can dimerize and functions as a transcription factor (FIG. 10A).

10.2. NK92 Cell culture: Culture of tumor cells was described in 6.1. NK-92 were cultured at 37° C. in 5% CO₂ in MEM alpha medium (Thermo Fisher #22561-021), 12.5% fetal bovine serum, Horse serum (12.5% v/v, Sigma #H1270-500ML), 2 mM L-glutamine and human recombinant IL-2 (5 ng/ml, Peprotech #200-02).

10.2. pSTAT5 measurement in NK-92 cells: 24 hours before the assay, NK-92 cells are washed 4 times in DPBS (Thermo Fisher, #14190-144) and cultured for 24 hours under the same conditions but without human recombinant IL-2.

10.2.1. FIGS. 10A-10F: NK-92 cells are washed in DPBS, resuspended at 5×10⁶ cells/ml in DPBS, Fc Block (30 μl/ml BD Biosciences, #564220) and Fixable Viability Stain 620 (1 μl/ml BD Biosciences, #564996) and incubated at room temperature in the dark for 15 min. NK-92 cells are washed once and stained for 20-30 min. at 4° C. at 2×10⁶ cells/ml with anti-human CD45-V500 (1 μl/100 μl BD Biosciences, #560777) in DPBS, Bovine Serum Albumin (2% m/v, Sigma #A3912-100G). NK-92 cells and harvested tumor cells are washed twice and transferred to 96-well round bottom plates at 150′000 cells/well each. Cells are incubated for 1-2h at 37° C. in 5% CO₂ under different test conditions in RPMI 1640 (Thermo Fisher, #11875-093), 10% fetal bovine serum, 2 mM L-glutamine, Hepes (10 mM, Sigma #H0887), non-essential amino acids (lx, Sigma #M7145-100 mL), β-mercaptoethanol (0.050 mM, Thermo Fisher #31350-010), Gentamicin (25 μg/ml, Sigma #G1397-10ML). Cells are fixed with 0.4% formaldehyde for 12 min. at 37° C. then washed and permeabilized in MetOH for 30 min. on ice. Wash once in DPBS and stain with Alexa Fluor 488 anti-STATS (pY694) (3 μl/well BD Biosciences, #562075) in DPBS, 2% Bovine Serum Albumine for 1 h at room temperature in the dark. Cells are washed twice with DPBS, 2% Bovine Serum Albumine and resuspend in 150 μl DPBS for flow cytometry analysis. The threshold for discriminating pSTAT5⁺ from pSTAT5⁻ cells is decided based on control non-stimulated wells. Data are provided as percentage of pSTAT5 positive cells.

In the presence of any of the HER2 over-expressing cells both the combinations of two IL-2R×HER2 bsAbs targeting two different HER2 epitopes (FIGS. 10B-10C) and the combinations targeting the same HER2 epitope (FIGS. 10D-10E) induced IL-2 signaling activation. Furthermore, we also observed activation of the pathway when using all four IL-2R×HER2 bsAbs concurrently (FIG. 10F). Importantly, the percentage of pSTAT5⁺ cells observed with some of the combinations of IL-2R×HER2 bsAbs was close to those induced by recombinant human IL-2 (FIGS. 10B-10F). The results confirm that, in the presence of HER2 over-expressing tumor cells, all combinations of KiH bsAbs tested can induce IL-2 signaling in non-transduced cells of immune origin.

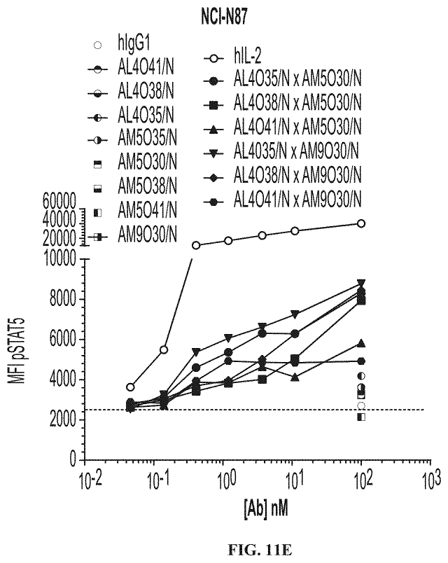

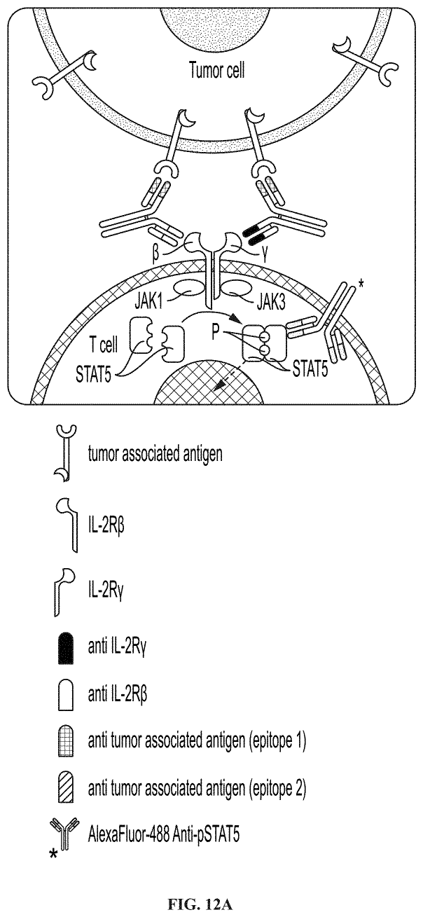

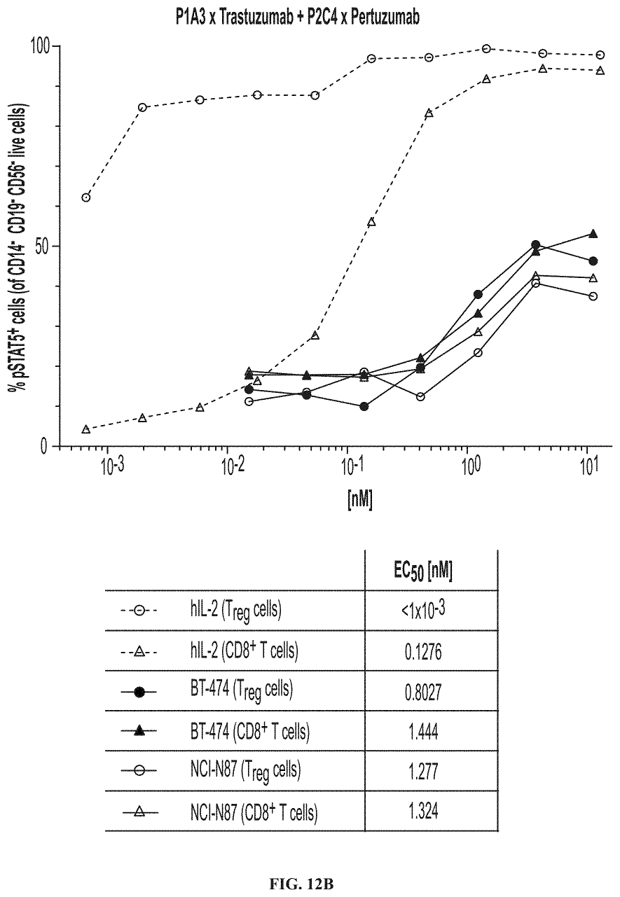

10.2.2. A similar finding was generated with various combinations of proprietary IL-2R×TAA κλ-bodies using a different protocol (FIGS. 11A-11E). The day of the assay, NK-92 and tumor cells are washed twice in DPBS and transferred to 96-well flat-bottom plates at 150′000 cells/well each. Cells are incubated for 30 min. at 37° C. in 5% CO₂, under different test conditions, in MEM alpha medium (Thermo Fischer #22561-021), 12.5% fetal bovine serum, horse serum (12.5% v/v, Sigma #H1270-500ML), 2 mM L-glutamine. After incubation, cells are transferred to V-bottom 96-well plates and washed before incubation, respectively, with Fixable Viability Stain 620 (1 μL/mL BD Biosciences, #564996) for 5 min. at 37° C., Fc Block (30 μL/mL BD Biosciences, #564220) for 10 min. at room temperature and anti-human CD45-V500 (1 μL/100 μL BD Biosciences, #560777) for 30 min. at 4° C. Cells are washed with DPBS, 2% Bovine Serum Albumin, between each step of incubation. Finally, cells are fixed with 0.4% formaldehyde for 12 min. at 37° C., then washed and permeabilized in MetOH for 30 min. on ice. After one wash, cells are stained with Alexa Fluor 488 anti-STATS (pY694) (3 μl/well BD Biosciences, #562075) in DPBS, 2% Bovine Serum Albumin, for 1 h at room temperature in the dark, and washed twice with DPBS, 2% Bovine Serum Albumin before being resuspended in 150 μl of DPBS for flow cytometry analysis. The threshold for discriminating pSTAT5⁺ from pSTAT5⁻ cells is decided based on control non-stimulated wells. Data are provided as Median Fluorescent Intensity (MFI) levels of pSTAT5 in CD45+ cells.

A first set of IL-2R×HER2 bsAbs was tested by combining κλ-bodies sharing the same HER2 arm (N2-28) with two different IL-2Rb arms (AL2 and AL4) and κλ-bodies sharing the same HER2 arm (N2-19, targeting another HER2 epitope than N2-28) with two different IL-2Rg arms (AM5 and AM9). All κλ-bodies were cross-combined and tested as depicted in FIG. 6A in presence of HER2-coated beads (fixed ratio 6:1, beads: IL-2 reporter cell line). Data showed pSTAT5 induction in NK92 cells regardless of the combination tested. IL-2 signaling is increased in presence of both HER2-overexpressing tumor cell lines: BT-474 (FIG. 11A) and NCI-N87 (FIG. 11B). IL-2 signaling induction is similar across all κλ-body combinations tested.

To demonstrate that IL-2R-bsAb combination can also mediate IL2 signaling in presence of MSLN⁺ tumor cells another set of IL-2R×MSLN κλ-bodies were tested in combination in presence of three MSLN-expressing cell lines (FIGS. 11C-11E). All combinations of bsAbs tested induced a similar increase in pSTAT5 in NK92 cells in presence of NCI-H226 (FIG. 11C) or OVCAR-3 (FIG. 11D). Interestingly, while expressing a weaker level of MSLN (24′000 MSLN/cell), NCI-N87 are also able to mediate IL-2 signaling regardless of the pair of IL-2R×MSLN κλ-bodies tested (FIG. 11E). Importantly, all κλ-bodies tested as single agents (i.e. not combined) do not induce IL-2 signaling, highlighting that IL-2Rb- and IL-2Rg-bsAbs are only active when combined (FIGS. 11C-11E).

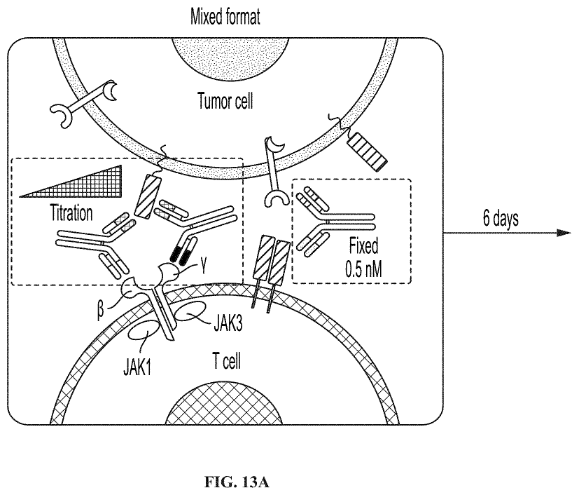

In the previous example, we showed that our IL-2R×TAA bsAb combination can induce IL-2 signaling in target non-transduced cells of immune origin. To extend this observation to primary human immune cells, we used human peripheral blood mononuclear cells (PBMCs). hIL-2 is known to preferentially activate regulatory T cells as they constitutively express the high affinity trimeric receptor. As our combination of bsAbs targets only the β and γ subunits of the IL-2R, we expect to observe a similar sensitivity of CD8⁺ T cells and regulatory T cells to our bsAbs. To test this hypothesis, we measured the level of pSTAT5 in different cell populations by flow cytometry (FIG. 12A).

10.3. PBMC isolation, stimulation and pSTAT5 staining: the protocol used was adapted from the one described in example 7. PBMCs cells are washed in DPBS, resuspended at 5×10⁶ cells/ml in DPBS, Fc Block (30 μl/ml BD Biosciences, #564220) and Fixable Viability Dye eFluor 506 (1 μl/ml Thermo Fisher, #65-0866-18) and incubated at room temperature in the dark for 15 min. PBMCs cells are washed once and stained for 20-30 min. at 4° C. at 2×10⁶ cells/ml with Alexa Fluor 594 anti-human CD3 Antibody (1 μl/100 μl, Biolegend, #300446), APC-Cy7 Mouse Anti-Human CD8 (1 μl/100 μl, BD Biosciences, #557834), BV421 Mouse Anti-Human CD4 (1 μl/100 μl, BD Biosciences, #562424)), eFluor 506 eBioscience CD19 Monoclonal Antibody (1 μl/100 μl, Thermo Fisher, #69-0199-42), eFluor 506 eBioscience CD14 Monoclonal Antibody (1 μl/100 μl, Thermo Fisher, #69-0149-42), eFluor 506 eBioscience CD56 (NCAM) Monoclonal Antibody (1 μl/100 μl, Thermo Fisher, #69-0566-42), in DPBS, 2% Bovine Serum Albumin. PBMC cells and harvested tumor cells are washed twice and transferred to 96-well round bottom plates at 250′000 PBMCs/well and 25′000 tumor cells/well. Cells are incubated for 1h at 37° C. in 5% CO₂ under different test conditions in RPMI 1640, 10% fetal bovine serum, 2 mM L-glutamine, 10 mM Hepes, lx non-essential amino, 0.050 mM β-mercaptoethanol, 25 μg/ml Gentamicin. Cells are resuspended in 100 μl/well Fix Buffer I (BD Biosciences, #557870) and incubated at 37° C. for 10 min. Cells are washed and permeabilized in pre-chilled 100 μl/well Perm Buffer III (BD Biosciences, #558050) for 30 min. on ice. Wash once in DPBS and stain with Alexa Fluor 488 anti-STATS (pY694) (3 μl/well BD Biosciences, #562075), Alexa Fluor 647 Mouse anti-Human FoxP3 (10 μl/100 μl, BD Biosciences, #560045) in DPBS, 2% Bovine Serum Albumine for 1 h at room temperature in the dark. Cells are washed twice with DPBS, 2% Bovine Serum Albumin and resuspend in 150 μl DPBS for flow cytometry analysis. The threshold for discriminating pSTAT5⁺ from pSTAT5⁻ cells is decided based on control non-stimulated wells.

Both hIL-2 and the IL-2R×HER2 KiH bsAb pairs in conjunction with HER2-overexpressing cells activated IL-2 signaling in T cells. Expectedly, regulatory T cells were significantly more sensitive to hIL-2 compared to CD8⁺ T cells as they remained activated at doses more than 100 times lower (FIG. 12B). Importantly, the difference in sensitivity between regulatory T cells and CD8⁺ T cells was abolished with our bsAb pair with both HER2-overexpressing tumor cells tested (FIG. 12B). These results show that our strategy, similarly to other approaches that avoid preferential engagement of trimeric IL-2 receptors, has an enhanced selectivity for immune stimulatory capacities.

### Example 11: T-Cell Dependent Cellular Cytotoxicity (TDCC) Mediated by IL-2R×TAA bsAbs Combined with a CD3×TAA bsAb

Costimulatory IL-2R×TAA bsAbs capable of triggering IL-2R signaling are expected to synergize with CD3×TAA bsAbs in enhancing T cell activation, which may translate into superior tumor cell killing in T-cell dependent cellular cytotoxicity (TDCC) assays. To avoid binding competition between CD3×TAA and IL-2R×TAA bsAbs, IL-2R×MSLN bsAb pairs were combined with CD3×HER2 bsAb, while IL-2R×MSLN bsAb pairs combined with CD3×HER2 bsAb in TDCC assays. NCI-N87 tumor cell line was selected as the target cells based on its dual expression of HER2 and MSLN.

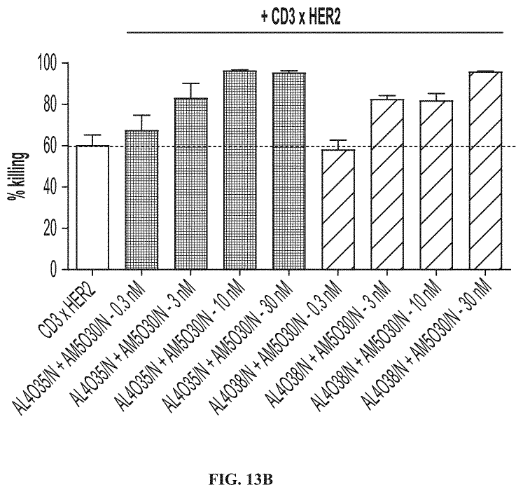

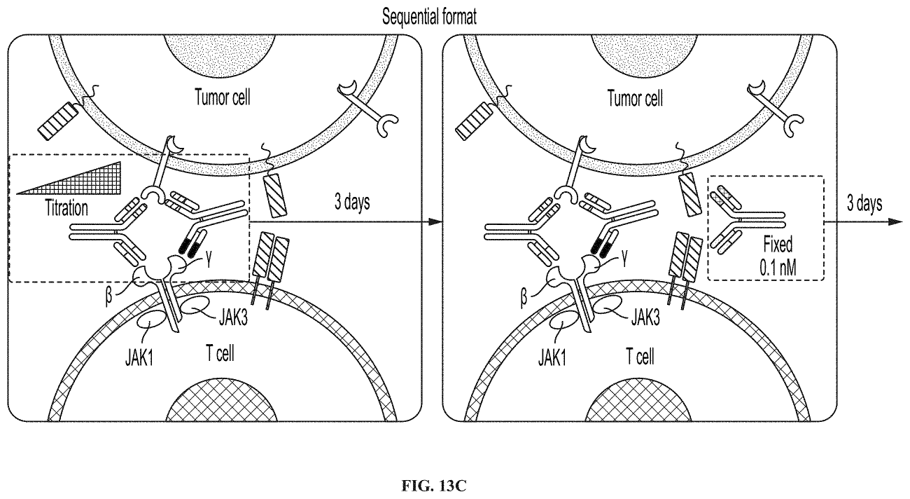

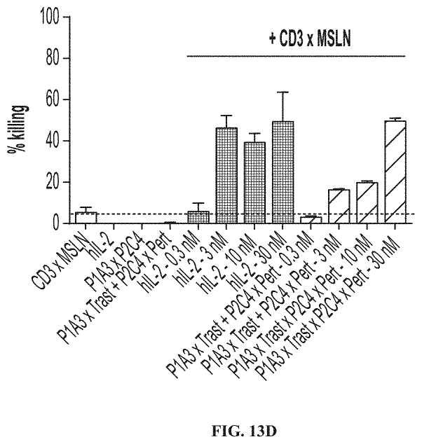

11.1. The TDCC assays: in vitro killing of the HER2/MSLN double-positive cell line, NCI-N87, induced by MSLN×CD3 or HER2×CD3 bsAbs was assessed in combination with IL-2R×HER2 bsAb pair or IL-2R×MSLN bsAb pair respectively. For the TDCC assay, PBMCs were added to target cells at final E:T ratio of 1:1. Target cells were detached with trypsin or cell dissociation solution after two washes with PBS. After a centrifugation step, cells were resuspended in assay media (RPMI 1640 (Thermo Fisher, #11875-093), 10% fetal bovine serum, 2 mM L-glutamine, Hepes (10 mM, Sigma #H0887), non-essential amino acids (1×, Sigma #M7145-100 mL), Sodium Pyruvate (1 mM, Sigma #58636-100 mL), β-mercaptoethanol (0.050 mM, Thermo Fisher #31350-010), Gentamicin (25 μg/ml, Sigma #G1397-10ML), adjusted to the needed concentration, and plated in 96-well plates. Effector cells were human peripheral blood mononuclear cells (PBMCs) isolated from buffy coats derived from healthy human donors using SepMate™ Tubes (Stemcell Technologies) with Lymphoprep™ buffer (Stemcell Technologies). A dose range of IL-2R×TAA bsAb pairs (0.3, 3, 10 and 30 nM) of the invention and a fixed dose of a CD3×TAA bsAbs (0.1 or 0.5 nM) were added to the pre-plated target and effector cells. As control, single-agent CD3×TAA bsAb was used. Target cell killing was assessed after 6 days of incubation at 37° C., 5% CO₂ by quantifying the number of viable adherent cells in culture using Promega's CellTiter-Glo® (G7570). Two different TDCC assay formats were tested: combining all bsAbs together at the same time for 6 days (FIG. 13A, referred as the mixed TDCC assay) or testing sequentially by first pre-stimulating T cells with IL-2R×TAA bsAb pairs for 3 days followed by the CD3×TAA bsAb for another 3 days (FIG. 13C, referred as the sequential TDCC assay). Combination of CD3×TAA with IL-2, instead of the IL-2R×TAA bsAbs was used as the positive control. CD3×TAA bsAbs single treatment was used as a reference comparator (dashed lines). hIgG1 or single treatment with IL-2R-bsAb pairs (e.g. in absence of the CD3×TAA bsAb) were used as negative controls.

In the mixed TDCC killing assay, two different pairs of IL-2R×MSLN bsAb combinations (AL4O35/N+AM5O30/N or AL4O38/N+AM5O30/N) were shown to enhance HER2×CD3 bsAb-induced killing of MSLN/HER2 double-positive NCI-N87 tumor target cells in dose-dependent manners (FIG. 13B). A similar synergy was observed using the sequential killing assay where a dose range of IL-2R×HER2 bsAb pair (P1A3× Trastuzumab+P2C4× Pertuzumab) was followed by a fixed dose of a CD3×MSLN bsAb. Importantly, the combination of CD3×MSLN with 30 nM of IL-2R×HER2 bsAbs was at least as potent as the combination of CD3×MSLN bsAb with IL-2 in inducing tumor cell killing (FIG. 13D). In the absence of the CD3×MSLN bsAb, no killing was induced by the IL-2R×HER2 bsAb pair alone (FIG. 13D).

Overall, TDCC data presented in the present invention demonstrates that IL-2Rβ×TAA and IL-2Rγ×TAA bsAb pair can increase TDCC mediated by a CD3×TAA bsAb to a level close to that induced by the combination of IL-2 and CD3×TAA bsAb.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
a) a first composition comprising a first bispecific antibody comprising an antigen binding domain that binds to a first tumor associated antigen and an antigen binding domain that binds to a first subunit of a cytokine receptor; and
b) a second composition comprising a bispecific antibody having an antigen binding domain that binds a second tumor associated antigen and an antigen binding domain that binds to a second subunit of the cytokine receptor.

2. The composition of claim 1, wherein the first tumor associated antigen and the second tumor associated antigen are different tumor associated antigens.

3. The composition of claim 1, wherein the first tumor associated antigen and the second tumor associated antigen are the same tumor associated antigen.

4. The composition of claim 1, wherein the antigen binding domain that binds to the first tumor associated antigen and the antigen binding domain that binds to the second tumor associated antigen, bind to two different epitopes of the same tumor associated antigen.

5. The composition of claim 1, wherein the antigen binding domain that binds to the first tumor associated antigen and the antigen binding domain that binds to the second tumor associated antigen are identical.

6. The composition of claim 1, wherein the first tumor associated antigen and the second tumor associated antigen are expressed on the surface of the same tumor cell.

7. The composition of claim 1, wherein the first subunit of a cytokine receptor and the second subunit of a cytokine receptor are expressed on the surface of the same immune cell.

8. The composition of claim 7, wherein the immune cell is a T-cell.

9. The composition of claim 1, wherein the first tumor associated antigen and/or the second tumor associated antigen is human epidermal growth factor receptor 2 (HER2).

10. The composition of claim 1, wherein the first tumor associated antigen and/or the second tumor associated antigen is mesothelin (MSLN).

11. The composition of claim 1, wherein the cytokine receptor binds IL-2, IL-4, IL-7, IL-9, IL-15, IL-21, IL-12, IL-23, IFNα, IFNβ, IFNε, IFNk, IFNo, IFNδ, IFNτ, IFNω̅, IFNζ, IFNγ or IFNλ.

12. The composition of claim 1, wherein the first subunit of the cytokine receptor or the second subunit of the cytokine receptor is a IL-2Rγ.

13. The composition of claim 1, wherein the first subunit of the cytokine receptor is IL-2Rβ and the second subunit of the cytokine receptor is IL-2Rγ.

14. The composition of claim 1, wherein the composition further comprises:
c) a third bispecific antibody comprising an antigen binding domain that binds to a third tumor associated antigen and an antigen binding domain that binds to an antigen expressed on a T-cell.

15. The composition of claim 14, wherein the third tumor associated antigen is different than the first tumor associated antigen and the second tumor associated antigen.

16. The composition of claim 14, wherein the antigen expressed on the T-cell is a CD3.

17. The composition of claim 14, wherein the first bispecific antibody, the second bispecific antibody and/or the third bispecific antibody has an IgG isotype.

18. The composition of claim 14, wherein the first bispecific antibody, the second bispecific antibody and/or the third bispecific antibody is a chimeric antibody, a humanized antibody or a human antibody.

19. The composition of claim 1, wherein the composition enables antigen dependent activation of the cytokine receptor.

20. The composition of claim 1, wherein the composition enables antigen dependent activation of IL-2 receptor signaling in immune cells expressing IL-2Rγ and IL-2Rβ.

21. The composition of claim 1, wherein the composition enables antigen dependent activation of IL-15 receptor signaling in immune cells expressing IL-2Rγ and IL-2Rβ.

22. A method of inhibiting tumor growth or progression in a subject in need thereof, comprising administering to the subject a therapeutically effective amount of the composition of claim 1.

23. A method of treating a cancer in a subject in need thereof, comprising administering to the subject a therapeutically effective amount of the composition of claim 1.

24. A method of enhancing T-cell mediated cell killing in a subject in need thereof, comprising administering to the subject a therapeutically effective amount of the composition of claim 1.

25. A method of enhancing T-cell activation in a subject in need thereof, comprising administering to the subject a therapeutically effective amount of the composition of claim 1.


SEQUENCE LISTING (184 sequences)

SEQ ID NO: 1 - protein, 8 aa, synthetic construct
GFTFSSYA

SEQ ID NO: 2 - protein, 8 aa, synthetic construct
ISGSGGST

SEQ ID NO: 3 - protein, 9 aa, synthetic construct
AKSYGAFDY

SEQ ID NO: 4 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 5 - protein, 445 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 108 aa, synthetic construct
QSALTQPASVSGSPGQSIAISCTGTSSDIGHYDFVSWYQQHPGTAPKLIIYDINNRPSGISNRFSGSKSDNMASLTISGLQPEDEADYYCSAYTSSDTLVFGGGTKLT

SEQ ID NO: 7 - protein, 14 aa, synthetic construct
TGTSSDIGHYDFVS

SEQ ID NO: 8 - protein, 7 aa, synthetic construct
DINNRPS

SEQ ID NO: 9 - protein, 10 aa, synthetic construct
SAYTSSDTLV

SEQ ID NO: 10 - protein, 121 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 11 - protein, 5 aa, synthetic construct
NYYMH

SEQ ID NO: 12 - protein, 17 aa, synthetic construct
AIMPSRGGTSYPQKFQG

SEQ ID NO: 13 - protein, 12 aa, synthetic construct
GEYYYDSSGYYY

SEQ ID NO: 14 - protein, 112 aa, synthetic construct
DVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 15 - protein, 16 aa, synthetic construct
RSSQSLLHSNGYNYLD

SEQ ID NO: 16 - protein, 7 aa, synthetic construct
LGSNRDS

SEQ ID NO: 17 - protein, 9 aa, synthetic construct
MQGTHWPWT

SEQ ID NO: 18 - protein, 120 aa, synthetic construct
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSS

SEQ ID NO: 19 - protein, 5 aa, synthetic construct
GYYWS

SEQ ID NO: 20 - protein, 16 aa, synthetic construct
EINHSGSTNYNPSLKS

SEQ ID NO: 21 - protein, 12 aa, synthetic construct
SPGGYSGGYFQH

SEQ ID NO: 22 - protein, 341 aa, synthetic construct
FGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWQSNGQTENNYKTTPPVLDSDGSFFLYSKLTVDKSRWQQGNVFSCSVMHEALHNHYTQKSLSLSPG

SEQ ID NO: 23 - protein, 11 aa, synthetic construct
RASQDVNTAVA

SEQ ID NO: 24 - protein, 6 aa, synthetic construct
SASFLY

SEQ ID NO: 25 - protein, 9 aa, synthetic construct
QQHYTTPPT

SEQ ID NO: 26 - protein, 116 aa, synthetic construct
WGQGTLVTVSSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 10 aa, synthetic construct
GFNIKDTYIH

SEQ ID NO: 28 - protein, 18 aa, synthetic construct
ARIYPTNGYTRYADSVKG

SEQ ID NO: 29 - protein, 13 aa, synthetic construct
SRWGGDGFYAMDY

SEQ ID NO: 30 - protein, 341 aa, synthetic construct; identical to SEQ ID NO: 22

SEQ ID NO: 31 - protein, 11 aa, synthetic construct
KASQDVSIGVA

SEQ ID NO: 32 - protein, 6 aa, synthetic construct
SASYRY

SEQ ID NO: 33 - protein, 9 aa, synthetic construct
QQYYIYPYT

SEQ ID NO: 34 - protein, 116 aa, synthetic construct; identical to SEQ ID NO: 26

SEQ ID NO: 35 - protein, 10 aa, synthetic construct
GFTFTDYTMD

SEQ ID NO: 36 - protein, 18 aa, synthetic construct
ADVNPNSGGSIYNQRFKG

SEQ ID NO: 37 - protein, 8 aa, synthetic construct
GPSFYFDY

SEQ ID NO: 38 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDANDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 6 aa, synthetic construct
QDISNY

SEQ ID NO: 40 - skipped

SEQ ID NO: 41 - protein, 9 aa, synthetic construct
QQDANDPRT

SEQ ID NO: 42 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDANDPRTFGQGTKVEIK

SEQ ID NO: 43 - protein, 216 aa, synthetic construct
DIQMTQSPSSLSASVGDRVTITCQASQDIGTYLNWYQQKPGKAPKLLIYEASTLETGVPSRFSGSGSGTDFTFTISSLQPEDIATYYCQQDGARDDYATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 6 aa, synthetic construct
QDIGTY

SEQ ID NO: 45 - skipped

SEQ ID NO: 46 - protein, 11 aa, synthetic construct
QQDGARDDYAT

SEQ ID NO: 47 - protein, 109 aa, synthetic construct
DIQMTQSPSSLSASVGDRVTITCQASQDIGTYLNWYQQKPGKAPKLLIYEASTLETGVPSRFSGSGSGTDFTFTISSLQPEDIATYYCQQDGARDDYATFGQGTKVEIK

SEQ ID NO: 48 - protein, 214 aa, synthetic construct
DIQMTQSPSSLSASVGDRVTITCQASQDIDDYLNWYQQKPGKAPKLLIYDASNLQSGVPSRFSGSGSGTDFTLTISSLQPEDFATYYCQQDSMDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 6 aa, synthetic construct
QDIDDY

SEQ ID NO: 50 - skipped

SEQ ID NO: 51 - protein, 9 aa, synthetic construct
QQDSMDPRT

SEQ ID NO: 52 - protein, 107 aa, synthetic construct
DIQMTQSPSSLSASVGDRVTITCQASQDIDDYLNWYQQKPGKAPKLLIYDASNLQSGVPSRFSGSGSGTDFTLTISSLQPEDFATYYCQQDSMDPRTFGQGTKVEIK

SEQ ID NO: 53 - protein, 216 aa, synthetic construct
DIQMTQSPSSLSASVGDRVTITCRASQSIDEYLNWYQQKPGKAPKLLIYEASKLQSGVPSRFSGSGSGTDFTLTISSLQPEDFATYYCQQDGAMDTYATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 6 aa, synthetic construct
QSIDEY

SEQ ID NO: 55 - skipped

SEQ ID NO: 56 - protein, 11 aa, synthetic construct
QQDGAMDTYAT

SEQ ID NO: 57 - protein, 109 aa, synthetic construct
DIQMTQSPSSLSASVGDRVTITCRASQSIDEYLNWYQQKPGKAPKLLIYEASKLQSGVPSRFSGSGSGTDFTLTISSLQPEDFATYYCQQDGAMDTYATFGQGTKVEIK

SEQ ID NO: 58 - protein, 214 aa, synthetic construct
EIVLTQSPATLSLSPGERATLSCRASQSVDEYLAWYQQKPGQAPRLLIYDASERATGIPARFSGSGSGTDFTLTISSLEPEDFAVYYCQQDATDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 6 aa, synthetic construct
QSVDEY

SEQ ID NO: 60 - skipped

SEQ ID NO: 61 - protein, 9 aa, synthetic construct
QQDATDPRT

SEQ ID NO: 62 - protein, 107 aa, synthetic construct
EIVLTQSPATLSLSPGERATLSCRASQSVDEYLAWYQQKPGQAPRLLIYDASERATGIPARFSGSGSGTDFTLTISSLEPEDFAVYYCQQDATDPRTFGQGTKVEIK

SEQ ID NO: 63 - protein, 216 aa, synthetic construct
DIQMTQSPSSLSASVGDRVTITCRASQSIYYYLNWYQQKPGKAPKLLIYDASALQSGVPSRFSGSGSGTDFTLTISSLQPEDFATYYCQQIDFTAGSITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 6 aa, synthetic construct
QSIYYY

SEQ ID NO: 65 - skipped

SEQ ID NO: 66 - protein, 11 aa, synthetic construct
QQIDFTAGSIT

SEQ ID NO: 67 - protein, 109 aa, synthetic construct
DIQMTQSPSSLSASVGDRVTITCRASQSIYYYLNWYQQKPGKAPKLLIYDASALQSGVPSRFSGSGSGTDFTLTISSLQPEDFATYYCQQIDFTAGSITFGQGTKVEIK

SEQ ID NO: 68 - protein, 215 aa, synthetic construct
DIQLTQSPSFLSASVGDRVTITCRASQTIDAPLRWYQQKPGKAPKLLIYLTSSLQSGVPSRFSGSGSGTEFTLTISSLQPEDFATYYCQQGYAAGPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 6 aa, synthetic construct
QTIDAP

SEQ ID NO: 70 - skipped

SEQ ID NO: 71 - protein, 10 aa, synthetic construct
QQGYAAGPST

SEQ ID NO: 72 - protein, 108 aa, synthetic construct
DIQLTQSPSFLSASVGDRVTITCRASQTIDAPLRWYQQKPGKAPKLLIYLTSSLQSGVPSRFSGSGSGTEFTLTISSLQPEDFATYYCQQGYAAGPSTFGQGTKVEIK

SEQ ID NO: 73 - protein, 214 aa, synthetic construct
DIQMTQSPSTLSASVGDTVTITCRASHYITTWLAWYQQKPGKAPKLLIYDVSSLESGVPSRFRGRGSGTEFTLTISSLQPDDFATYYCQQYESYSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 6 aa, synthetic construct
HYITTW

SEQ ID NO: 75 - skipped

SEQ ID NO: 76 - protein, 9 aa, synthetic construct
QQYESYSPT

SEQ ID NO: 77 - protein, 107 aa, synthetic construct
DIQMTQSPSTLSASVGDTVTITCRASHYITTWLAWYQQKPGKAPKLLIYDVSSLESGVPSRFRGRGSGTEFTLTISSLQPDDFATYYCQQYESYSPTFGQGTKVEIK

SEQ ID NO: 78 - protein, 215 aa, synthetic construct
DIQMTQSPSTLSASVGDRVTITCRASQTIYGPLNWYQQKPGKAPKLLIYSTSYLESGVPSRFSGSGSGTEFTLTISSLQPDDFATYYCQQAGYASAPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 6 aa, synthetic construct
QTIYGP

SEQ ID NO: 80 - skipped

SEQ ID NO: 81 - protein, 10 aa, synthetic construct
QQAGYASAPT

SEQ ID NO: 82 - protein, 108 aa, synthetic construct
DIQMTQSPSTLSASVGDRVTITCRASQTIYGPLNWYQQKPGKAPKLLIYSTSYLESGVPSRFSGSGSGTEFTLTISSLQPDDFATYYCQQAGYASAPTFGQGTKVEIK

SEQ ID NO: 83 - protein, 219 aa, synthetic construct
DIVMTQSPDSLAVSLGERATINCKSSQSVLYSEVAYTALAWYQQKPGQPPKLLIYATSTRESGVPDRFSGSGSGTDFTLTISSLQAEDVAVYYCQQGYGHPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 12 aa, synthetic construct
QSVLYSEVAYTA

SEQ ID NO: 85 - skipped

SEQ ID NO: 86 - protein, 8 aa, synthetic construct
QQGYGHPT

SEQ ID NO: 87 - protein, 112 aa, synthetic construct
DIVMTQSPDSLAVSLGERATINCKSSQSVLYSEVAYTALAWYQQKPGQPPKLLIYATSTRESGVPDRFSGSGSGTDFTLTISSLQAEDVAVYYCQQGYGHPTFGQGTKVEIK

SEQ ID NO: 88 - protein, 221 aa, synthetic construct
DIVMTQSPDSLAVSLGERATINCKSSQSVLYDDFGNANLAWYQQKPGQPPKLLIYYGSYRESGVPDRFSGSGSGTDFTLTISSLQAEDVAVYYCQQVDVGLAITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 12 aa, synthetic construct
QSVLYDDFGNAN

SEQ ID NO: 90 - skipped

SEQ ID NO: 91 - protein, 10 aa, synthetic construct
QQVDVGLAIT

SEQ ID NO: 92 - protein, 114 aa, synthetic construct
DIVMTQSPDSLAVSLGERATINCKSSQSVLYDDFGNANLAWYQQKPGQPPKLLIYYGSYRESGVPDRFSGSGSGTDFTLTISSLQAEDVAVYYCQQVDVGLAITFGQGTKVEIK

SEQ ID NO: 93 - protein, 213 aa, synthetic construct
DIQLTQSPSFLSASVGDRVTITCRASQDIGIELAWYQQKPGKAPKLLIYFESHLQSGVPSRFSGSGSGTEFTLTISSLQPEDFATYYCQQIRIDPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 6 aa, synthetic construct
QDIGIE

SEQ ID NO: 95 - skipped

SEQ ID NO: 96 - protein, 8 aa, synthetic construct
QQIRIDPT

SEQ ID NO: 97 - protein, 106 aa, synthetic construct
DIQLTQSPSFLSASVGDRVTITCRASQDIGIELAWYQQKPGKAPKLLIYFESHLQSGVPSRFSGSGSGTEFTLTISSLQPEDFATYYCQQIRIDPTFGQGTKVEIK

SEQ ID NO: 98 - protein, 216 aa, synthetic construct
EIVLTQSPGTLSLSPGERATLSCRASQDVATRGLAWYQQKPGQAPRLLIYGASSRATGIPDRFSGSGSGTDFTLTISRLEPEDFAVYYCQQYELEHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 7 aa, synthetic construct
QDVATRG

SEQ ID NO: 100 - skipped

SEQ ID NO: 101 - protein, 10 aa, synthetic construct
QQYELEHPAT

SEQ ID NO: 102 - protein, 109 aa, synthetic construct
EIVLTQSPGTLSLSPGERATLSCRASQDVATRGLAWYQQKPGQAPRLLIYGASSRATGIPDRFSGSGSGTDFTLTISRLEPEDFAVYYCQQYELEHPATFGQGTKVEIK

SEQ ID NO: 103 - protein, 214 aa, synthetic construct
DIQMTQSPSSLSASVGDRVTITCQASQDIAGYLNWYQQKPGKAPKLLIYTASTLETGVPSRFSGSGSGTDFTFTISSLQPEDIATYYCQQWAFGPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 6 aa, synthetic construct
QDIAGY

SEQ ID NO: 105 - skipped

SEQ ID NO: 106 - protein, 9 aa, synthetic construct
QQWAFGPVT

SEQ ID NO: 107 - protein, 107 aa, synthetic construct
DIQMTQSPSSLSASVGDRVTITCQASQDIAGYLNWYQQKPGKAPKLLIYTASTLETGVPSRFSGSGSGTDFTFTISSLQPEDIATYYCQQWAFGPVTFGQGTKVEIK

SEQ ID NO: 108 - protein, 214 aa, synthetic construct
EIVLTQSPATLSLSPGERATLSCRASQSVFANLNWYQQKPGQAPRLLIYDSSGRATGIPARFSGSGSGTDFTLTISSLEPEDFAVYYCQQGFGPSLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 6 aa, synthetic construct
QSVFAN

SEQ ID NO: 110 - skipped

SEQ ID NO: 111 - protein, 9 aa, synthetic construct
QQGFGPSLT

SEQ ID NO: 112 - protein, 107 aa, synthetic construct
EIVLTQSPATLSLSPGERATLSCRASQSVFANLNWYQQKPGQAPRLLIYDSSGRATGIPARFSGSGSGTDFTLTISSLEPEDFAVYYCQQGFGPSLTFGQGTKVEIK

SEQ ID NO: 113 - protein, 215 aa, synthetic construct
EIVLTQSPGTLSLSPGERATLSCRASQNVNHNFLTWYQQKPGQAPRLLIYSASARATGIPDRFSGSGSGTDFTLTISRLEPEDFAVYYCQQFNYAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 7 aa, synthetic construct
QNVNHNF

SEQ ID NO: 115 - skipped

SEQ ID NO: 116 - protein, 9 aa, synthetic construct
QQFNYAPLT

SEQ ID NO: 117 - protein, 108 aa, synthetic construct
EIVLTQSPGTLSLSPGERATLSCRASQNVNHNFLTWYQQKPGQAPRLLIYSASARATGIPDRFSGSGSGTDFTLTISRLEPEDFAVYYCQQFNYAPLTFGQGTKVEIK

SEQ ID NO: 118 - protein, 125 aa, synthetic construct
EVQLVESGGGLVQPGGSLKLSCAASGNTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSS

SEQ ID NO: 119 - protein, 109 aa, synthetic construct
QTVVTQEPSLTVSPGGTVTLTCGSSTGAVTHGNYPNWVQQKPGQAPRGLIGGTKVLAPGTPARFSGSLLGGKAALTLSGVQPEDEAEYYCVLWYSNRWVFGGGTKLTVL

SEQ ID NO: 120 - protein, 116 aa, synthetic construct
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 121 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 122 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 123 - protein, 120 aa, synthetic construct [sequence omitted]

SEQ ID NO: 124 - protein, 109 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGGGTKLTVLSS

SEQ ID NO: 125 - protein, 218 aa, synthetic construct
QSVLTQPPSASGTPGQRVTISCSGSSSNIAHGPVNWYQQLPGTAPKLLIYATNHRPSGVPDRFSGSKSGTTASLTISGLQSEDEADYYCAAYDLTGWFAYAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 126 - protein, 8 aa, synthetic construct
SSNIAHGP

SEQ ID NO: 127 - skipped

SEQ ID NO: 128 - protein, 13 aa, synthetic construct
AAYDLTGWFAYAV

SEQ ID NO: 129 - protein, 123 aa, synthetic construct
QSVLTQPPSASGTPGQRVTISCSGSSSNIAHGPVNWYQQLPGTAPKLLIYATNHRPSGVPDRFSGSKSGTTASLTISGLQSEDEADYYCAAYDLTGWFAYAVFGGGTKLTVLGQPKAAPSVTL

SEQ ID NO: 130 - protein, 224 aa, synthetic construct
QPVLTQPVSLSASPGASVSLTCTLRSDIRVRDYRIFWYQQKPGSPPQYLLRYKTDSDKQQGSGVPSRFSGSKDASANAGILLISGLQSEDEADYYCMIWHRTTGTSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 131 - protein, 9 aa, synthetic construct
SDIRVRDYR

SEQ ID NO: 132 - protein, 7 aa, synthetic construct
YKTDSDK

SEQ ID NO: 133 - protein, 12 aa, synthetic construct
MIWHRTTGTSLV

SEQ ID NO: 134 - protein, 118 aa, synthetic construct
QPVLTQPVSLSASPGASVSLTCTLRSDIRVRDYRIFWYQQKPGSPPQYLLRYKTDSDKQQGSGVPSRFSGSKDASANAGILLISGLQSEDEADYYCMIWHRTTGTSLVFGGGTKLTVL

SEQ ID NO: 135 - protein, 225 aa, synthetic construct
QPVLTQPASLSASPGASASLTCTLRSGINVRDYRIFWYQQKPGSPPQYLLRYKSASDKQQGSGVPSRFSGSKDASANAGILLISGLQSEDEADYYCMIWHHDSEGHAFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 136 - protein, 9 aa, synthetic construct
SGINVRDYR

SEQ ID NO: 137 - protein, 7 aa, synthetic construct
YKSASDK

SEQ ID NO: 138 - protein, 13 aa, synthetic construct
MIWHHDSEGHAFV

SEQ ID NO: 139 - protein, 119 aa, synthetic construct
QPVLTQPASLSASPGASASLTCTLRSGINVRDYRIFWYQQKPGSPPQYLLRYKSASDKQQGSGVPSRFSGSKDASANAGILLISGLQSEDEADYYCMIWHHDSEGHAFVFGGGTKLTVL

SEQ ID NO: 140 - protein, 215 aa, synthetic construct
SYVLTQPPSVSVAPGKTARITCGGNKIGHRAVHWYQQKPGQAPVLVIYYTYERPSGIPERFSGSNSGNTATLTISRVEAGDEADYYCQVWDWYSEGGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 141 - protein, 6 aa, synthetic construct
KIGHRA

SEQ ID NO: 142 - skipped

SEQ ID NO: 143 - protein, 12 aa, synthetic construct
QVWDWYSEGGVV

SEQ ID NO: 144 - protein, 120 aa, synthetic construct
SYVLTQPPSVSVAPGKTARITCGGNKIGHRAVHWYQQKPGQAPVLVIYYTYERPSGIPERFSGSNSGNTATLTISRVEAGDEADYYCQVWDWYSEGGVVFGGGTKLTVLGQPKAAPSVTL

SEQ ID NO: 145 - protein, 217 aa, synthetic construct
QSVLTQPPSVSGAPGQRVTISCTGSSSNIENTHTVHWYQQLPGTAPKLLIYDASIRPSGVPDRFSGSKSGTSASLAITGLQAEDEADYYCQSYDWLRGAQVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 146 - protein, 9 aa, synthetic construct
SSNIENTHT

SEQ ID NO: 147 - skipped

SEQ ID NO: 148 - protein, 11 aa, synthetic construct
QSYDWLRGAQV

SEQ ID NO: 149 - protein, 111 aa, synthetic construct
QSVLTQPPSVSGAPGQRVTISCTGSSSNIENTHTVHWYQQLPGTAPKLLIYDASIRPSGVPDRFSGSKSGTSASLAITGLQAEDEADYYCQSYDWLRGAQVFGGGTKLTVL

SEQ ID NO: 150 - protein, 216 aa, synthetic construct
SYVLTQPPSVSVAPGKTARITCGGNTIGSTVVHWYQQKPGQAPVLVIYFDDARPSGIPERFSGSNSGNTATLTISRVEAGDEADYYCQVWDSFGLSLAPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 151 - protein, 6 aa, synthetic construct
TIGSTV

SEQ ID NO: 152 - skipped

SEQ ID NO: 153 - protein, 13 aa, synthetic construct
QVWDSFGLSLAPV

SEQ ID NO: 154 - protein, 110 aa, synthetic construct
SYVLTQPPSVSVAPGKTARITCGGNTIGSTVVHWYQQKPGQAPVLVIYFDDARPSGIPERFSGSNSGNTATLTISRVEAGDEADYYCQVWDSFGLSLAPVFGGGTKLTVL

SEQ ID NO: 155 - protein, 5 aa, synthetic construct
TYAMH

SEQ ID NO: 156 - protein, 17 aa, synthetic construct
WINTGNGNTKYSQNFQG

SEQ ID NO: 157 - protein, 11 aa, synthetic construct
DLGQLERLYFW

SEQ ID NO: 158 - protein, 11 aa, synthetic construct
RAGQAISSWLA

SEQ ID NO: 159 - protein, 7 aa, synthetic construct
KASNLES

SEQ ID NO: 160 - protein, 9 aa, synthetic construct
QQYQSYPYT

SEQ ID NO: 161 - protein, 119 aa, synthetic construct
EVQLVQSGTEVKKPGASVKVSCKASGYTFTTYAMHWVRQAPGQSLEWMGWINTGNGNTKYSQNFQGRVTMTRDTSISTAYMELSRLRSDDTAVYYCARDLGQLERLYFWGQGTLVTVSS

SEQ ID NO: 162 - protein, 107 aa, synthetic construct
DIQMTQSPSTLSASVGDRVTLSCRAGQAISSWLAWYQQKPGKAPKLLIYKASNLESGVPSRFSGGGSGAEFTLTISSLQPDDFATYYCQQYQSYPYTFGQGTKLEIR

SEQ ID NO: 163 - protein, 5 aa, synthetic construct
SYAMS

SEQ ID NO: 164 - protein, 17 aa, synthetic construct
AISGSGGSTYYADSVKG

SEQ ID NO: 165 - protein, 5 aa, synthetic construct
DLGDY

SEQ ID NO: 166 - protein, 11 aa, synthetic construct
QASQDIGNYLN

SEQ ID NO: 167 - protein, 7 aa, synthetic construct
DASNLET

SEQ ID NO: 168 - protein, 9 aa, synthetic construct
LQLYDYPLT

SEQ ID NO: 169 - protein, 114 aa, synthetic construct
HVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARDLGDYWGQGTLVTVSS

SEQ ID NO: 170 - protein, 107 aa, synthetic construct
DIQLTQSPSSLSASVGDRVTITCQASQDIGNYLNWYQLKPGKAPKLLIYDASNLETGVPSRFSGSGSGTDFTFTISSLQPEDIATYYCLQLYDYPLTFGGGTKVEIK

SEQ ID NO: 171 - protein, 8 aa, synthetic construct
SSSGDAFD

SEQ ID NO: 172 - protein, 13 aa, synthetic construct
TRSSGSIASNYVQ

SEQ ID NO: 173 - protein, 7 aa, synthetic construct
DDNQRPT

SEQ ID NO: 174 - protein, 9 aa, synthetic construct
QSSHSTAVV

SEQ ID NO: 175 - protein, 117 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSSSGDAFDIWGQGTMVTVSS

SEQ ID NO: 176 - protein, 110 aa, synthetic construct
NFMLTQPHSVSESPGKTVTISCTRSSGSIASNYVQWYQQRPGSSPTTVIFDDNQRPTGVPDRFSAAIDTSSSSASLTISGLTAEDEADYYCQSSHSTAVVFGGGTKLTVL

SEQ ID NO: 177 - protein, 120 aa, synthetic construct
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 178 - protein, 112 aa, synthetic construct; identical to SEQ ID NO: 14

SEQ ID NO: 179 - protein, 120 aa, synthetic construct
QVQLQQWGAGMLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 180 - protein, 112 aa, synthetic construct; identical to SEQ ID NO: 14

SEQ ID NO: 181 - protein, 120 aa, synthetic construct; identical to SEQ ID NO: 177

SEQ ID NO: 182 - protein, 112 aa, synthetic construct; identical to SEQ ID NO: 14

SEQ ID NO: 183 - protein, 120 aa, synthetic construct; identical to SEQ ID NO: 177

SEQ ID NO: 184 - protein, 112 aa, synthetic construct
DVVMTQSPLSLPVTPGESVSISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK